(19) United States
(12) Patent Application Publication
(10) Pub. No.: US 2009/0226888 A1
(43) Pub. Date: Sep. 10, 2009

(54) Diagnostic Primers And Method For Detecting Avian Influenza Virus Subtype H5 And H5N1

(75) Inventors: Ee Chee Ren, Singapore (SG); Lisa Fong Poh Ng, Singapore (SG); Jer Ming Chia, Singapore (SG)

Correspondence Address: KLARQUIST SPARKMAN, LLP, 121 SW SALMON STREET, SUITE 1600, PORTLAND, OR, 97204, US

(73) Assignee: AGENCY FOR SCIENCE, TECHNOLOGY AND RESEARCH, Singapore (SG)

(21) Appl. No.: 11/916,994

(22) PCT Filed: Feb. 14, 2006
(86) PCT No.: PCT/SG2006/000027

§ 371 Date: Nov. 13, 2008

Related U.S. Application Data
(60) Provisional application No. 60/578,353, filed on Jun. 10, 2004.

(30) Foreign Application Priority Data
Jun. 10, 2005 (SG) PCT/SG2005/000187

(51) Int. Cl. C12Q 1/70; C07H 21/00; G01N 33/566; C40B 40/06

(52) U.S. Cl. 435/5; 536/24.33; 436/501; 506/16

(57) ABSTRACT

The present invention provides primers directed to conserved regions of the HA and NA genes of avian influenza virus subtypes H5 and H5N1, and provides a method for detecting avian influenza subtype H5 or H5N1.

### CROSS-REFERENCE TO RELATED APPLICATION

This application is a continuation-in-part of international application PCT/SG2005/000187, filed on Jun. 10, 2005, which claims benefit and priority from U.S. provisional patent application No. 60/578,353, filed on Jun. 10, 2004, the contents of both of which applications are incorporated herein by reference.

### FIELD OF THE INVENTION

The present invention relates to a nucleic acid based detection method, more particularly, to primers and a method of detecting avian influenza virus.

### BACKGROUND OF THE INVENTION

Three types of influenza viruses, types A, B, and C are known and they belong to a family of single-stranded negative-sense enveloped RNA viruses called Orthomyxoviridae (Swayne, D. E., and D. L. Suarez (2000) Rev. Sci. Tech. 19:463-482). The viral genome is approximately 12 000 to 15 000 nucleotides in length and comprises eight RNA segments (seven in Type C).

Influenza A virus infects many animals such as humans, pigs, horses, marine mammals, and birds (Nicholson, K. G., et al. (2005) Lancet 362:1733-1745). Its natural reservoir is in aquatic birds, and in avian species most influenza virus infections cause mild localized infections of the respiratory and intestinal tract. However, the virus can have high pathogenic effect in poultry, with sudden outbreaks causing high mortality rates in affected poultry populations. Highly pathogenic strains such as H5N1 cause system infections in which mortality may reach 100% (Zeitlin, G. A., and M. J. Maslow (2005) Curr. Infect. Dis. Rep. 7:193-199). In humans, influenza viruses cause a highly contagious acute respiratory disease that have resulted in epidemic and pandemic disease in humans (Cox, N.J., and K. Subbarao (1999) Lancet 354:1277-1282).

Influenza A viruses can be classified into subtypes based on allelic variations in antigenic regions of two genes that encode surface glycoproteins, namely, hemagglutinin (HA) and neuraminidase (NA) which are required for viral attachment and cellular release. Other major viral proteins include the nucleoprotein, the nucleocapsid structural protein, membrane proteins (M1 and M2), polymerases (PA, PB1 and PB2), and non-structural proteins (NS1 and NS2). Currently, fifteen subtypes of HA (H1-H15) and nine NA (N1-N9) antigenic variants are known in influenza A virus. Subtypes H5 and H7 can cause highly pathogenic infections in poultry and certain subtypes have been shown to cross the species barrier to humans. Previously, only three subtypes have been known to circulate in humans (H1N1, H1N2, and H3N2). However, in recent years, the pathogenic H5N1 subtype of avian influenza A has been reported to cross the species barrier and infect humans as documented in Hong Kong in 1997 and 2003 (Peiris, J. S. M., et al. (2004) Lancet 363:617-619; Yuen, K. Y., et al. (1998) Lancet 351: 467-471), leading to the death of some patients. Since late 2003, the H5N1 avian influenza A in poultry reached epidemic proportions with reports of serious outbreaks in several Asian countries including Vietnam, Thailand, South Korea, Laos, Cambodia, Indonesia, Japan and Malaysia (Centers for Disease Control and Prevention (CDC) (2004) Morb. Mortal Wkly. Rep. 53:100-3; Hien T. T., et al. (2004) N. Engl. J. Med. 350:1179-1188) that resulted in massive culling of millions of poultry which had severe economic repercussions.

In humans, the avian influenza virus infects cells of the respiratory tract as well as the intestinal tract, liver, spleen, kidneys and other internal organs. Symptoms of avian flu infection include fever, respiratory difficulties including shortness of breath and cough, lymphopenia, diarrhea and difficulties regulating blood sugar levels. Due to the high pathogenicity of H5 subtypes, particularly H5N1, and their demonstrated ability to cross over to infect humans, there is a significant economic and public health risk associated with these viral strains, including a real epidemic and pandemic threat.

As a result, H5N1 avian influenza A virus represents a potential danger to human health not only in Asia but to the world. In addition to containment procedures, sensitive detection assays for early diagnosis are vital to lower the chances of spread and reduce the risk of development into an epidemic. Currently, there are a variety of techniques that can be used to detect H5 and H5N1 subtypes of avian influenza virus in biological samples, including nucleic acid sequence-based amplification (NASBA) methods that amplify RNA, viral cultures, reverse-transcription polymerase chain reaction (RT-PCR) methods that amplify DNA transcribed from the viral RNA genome, hemagglutination inhibition and various fluorescence and enzyme-linked immunoassays (ELISAs).

In particular, PCT publication WO 02/29118 by So et al. describes a NASBA assay and kit for detecting H5 subtypes of avian flu virus. Hien et al. (2004, New Eng. J of Med. 350(12):1179-1188) describe the use of antigen tests using various fluorescence and enzyme-linked immunoassays. Lau et al. (2003, Biochem. Biophys. Res. Comm. 313:336-342) describes a NASBA method for detection of H5 or H7 subtypes of avian influenza virus. Lee et al. (2001, J. Virol. Methods 97:13-32) and Payungporn et al. (2004, Viral Immunol. 17:588-593) describe RT-PCR assays for identification and subtyping or detection of avian flu virus subtypes. However, each of these methods uses genetic information derived from only a few isolates or variants of H5 or H5N1 to confirm the presence of virus. Furthermore, these assays are reported to be low in specificity and sensitivity. Clinically, the low sensitivity of these diagnostics may limit the usefulness for reliable detection of influenza A (H5N1) virus in humans. Therefore, there is an urgent need for sensitive diagnostic tests useful for rapid and early diagnosis.

### SUMMARY OF THE INVENTION

Based on sequence comparison of the HA gene from greater than 200 H5 isolates and greater than 100 H5N1 isolates, and on sequence comparison of the NA gene from approximately 70 H5N1 isolates, a series of primers directed to conserved regions within these genes has been developed. These primers are useful to screen for a wide variety of H5 and H5N1 isolates, and allow for detection methods that are rapid, specific and sensitive.

Thus, in one aspect, the present invention provides a primer comprising a sequence of any one of SEQ ID NO:1 to SEQ ID NO:114. In another aspect, there is provided a primer comprising a target annealing sequence and a non-influenza A virus sequence, wherein the target annealing sequence comprises a sequence of any one of SEQ ID NO:1 to SEQ ID NO:114. In a further aspect, there is provided a primer comprising the sequence of SEQ ID NO:118. In yet a further aspect, there is provided a primer comprising a target annealing sequence and a non-influenza A virus sequence, wherein the target annealing sequence comprises the sequence of SEQ ID NO:118.

These primers are useful for detecting the presence of avian influenza virus H5 or H5N1 in a sample, for example a sample derived from an organism suspected of carrying such a virus, and may be used in a reverse-transcription polymerase chain reaction in order to detect the presence of virus in the sample. Thus, in another aspect the present invention provides a method for detecting influenza A virus subtype H5 or H5N1 in a sample comprising amplifying DNA reverse transcribed from RNA obtained from the sample using one or more primers each comprising a sequence of any one of SEQ ID NO:1 to SEQ ID NO:114; and detecting a product of amplification, wherein the presence of the product of amplification indicates the presence of an avian influenza virus subtype H5 or H5N1 in the sample. In a further aspect, there is provided a method for detecting influenza A virus subtype H5N1 in a sample comprising amplifying DNA reverse transcribed from RNA obtained from the sample using a primer comprising the sequence of SEQ ID NO: 118; and detecting a product of amplification, wherein the presence of the product of amplification indicates the presence of an avian influenza virus subtype H5N1 in the sample. The methods described herein can be used to detect a wide variety of H5 and H5N1 influenza A virus isolates. Using a one-step method, in which RNA is reverse-transcribed and product is amplified in a single reaction tube, allows for a reduction in detection time, minimizes sample manipulation and lowers the risk of cross-contamination of samples. Thus, the described methods using the described primers may be useful for early detection and/or diagnosis of H5 and H5N1 influenza A infection. Furthermore, these methods can be used to determine approximate viral load in a sample, which application is useful in clinical and public health management settings.

The primers of the invention may be useful in other amplification methods, such as nucleic acid based sequence amplification methods to detect the presence of avian influenza virus subtype H5 or H5N1 in a sample. The primers of the invention may also be useful for sequencing DNA corresponding to the HA or NA gene of avian influenza virus subtype H5 or H5N1. In another aspect, there is provided a method of detecting influenza A virus subtype H5 or H5N1 in a sample comprising contacting the sample with a primer immobilized on a support, said primer comprising a sequence of any one of SEQ ID NO:1 to SEQ ID NO:114, under conditions suitable for hybridizing the primer and the sample; and detecting hybridization of the immobilized primer and the sample. In yet another aspect, there is provided a method of detecting influenza A virus subtype H5N1 in a sample comprising contacting the sample with a primer immobilized on a support, said primer comprising the sequence of SEQ ID NO:118, under conditions suitable for hybridizing the primer and the sample; and detecting hybridization of the primer and the sample.

In a further aspect, there is provided a method of detecting influenza A virus subtype H5 or H5N1 in a sample comprising contacting the sample with a nucleic acid microarray, the nucleic acid microarray comprising one or more primers, each of said primers comprising a sequence of any one of SEQ ID NO:1 to SEQ ID NO:114, under conditions suitable for hybridizing the one or more primers and the sample; and detecting hybridization of the one or more primers and the sample. In still a further aspect, there is provided a method of detecting influenza A virus subtype H5N1 in a sample comprising contacting the sample with a nucleic acid microarray, the nucleic acid microarray comprising one or more primers, one of said one or more primers comprising the sequence of SEQ ID NO:118, under conditions suitable for hybridizing the one or more primers and the sample; and detecting hybridization of the one or more primers and the sample.

In another aspect, there is provided a nucleic acid microarray comprising a primer, said primer comprising a sequence of any one of SEQ ID NO:1 to SEQ ID NO:114. In yet another aspect, there is provided a nucleic acid microarray comprising a primer, said primer comprising the sequence of SEQ ID NO:118.

In a further aspect, there is provided a kit comprising a primer as defined herein and instructions for detecting influenza A virus subtype H5 or H5N1 in a sample.

Other aspects and features of the present invention will become apparent to those of ordinary skill in the art upon review of the following description of specific embodiments of the invention in conjunction with the accompanying figures.

### BRIEF DESCRIPTION OF THE DRAWINGS

FIG. 1 is a schematic diagram representing the HA gene, and depicting the location of exemplary forward and reverse primers of the present invention (beginning with “gisAF”) and of primers known in the art (beginning with “TW”, “VM” or “HK”);

FIG. 2 is a photograph of an agarose gel displaying PCR amplification products prepared by a gel-based PCR approach using exemplary primers (sets 1 to 8) of the invention to amplify template DNA reverse transcribed from RNA of an H5N1 isolate;

FIG. 3 is a photograph of an agarose gel displaying the relative amounts of amplification product obtained using varying amounts of template and primer set 3 used in FIG. 2;

FIG. 4 is a photograph of an agarose gel displaying the relative amounts of amplification product obtained using varying amounts of template and primer set 5 used in FIG. 2;

FIG. 5 is a photograph of an agarose gel displaying the relative amounts of amplification product obtained using varying amounts of template and primer sets 8 (upper bands) and 6 (lower bands) used in FIG. 2;

FIG. 6 is a photograph of an agarose gel displaying PCR amplification products prepared by a real time PCR approach with SYBR green dye, using exemplary primers (sets 1 to 8) of the invention to amplify template DNA reverse transcribed from RNA of an H5N1 isolate;

FIG. 7 is an amplification curve obtained during the real time PCR amplification reaction using primer set 1 of FIG. 6;

FIG. 8 is an amplification curve obtained during the real time PCR amplification reaction using primer set 2 of FIG. 6;

FIG. 9 is an amplification curve obtained during the real time PCR amplification reaction using primer set 3 of FIG. 6;

FIG. 10 is an amplification curve obtained during the real time PCR amplification reaction using primer set 4 of FIG. 6;

FIG. 11 is an amplification curve obtained during the real time PCR amplification reaction using primer set 5 of FIG. 6;

FIG. 12 is an amplification curve obtained during the real time PCR amplification reaction using primer set 6 of FIG. 6;

FIG. 13 is an amplification curve obtained during the real time PCR amplification reaction using primer set 7 of FIG. 6;

FIG. 14 is an amplification curve obtained during the real time PCR amplification reaction using primer set 8 of FIG. 6;

FIG. 15 is a melting curve obtained at the end of the real time PCR amplification reaction using primer sets 1 and 2 of FIG. 6;

FIG. 16 is a melting curve obtained at the end of the real time PCR amplification reaction using primer sets 3, 4 and 5 of FIG. 6;

FIG. 17 is a melting curve obtained at the end of the real time PCR amplification reaction using primer sets 5 and 6 of FIG. 6;

FIG. 18 is a melting curve obtained at the end of the real time PCR amplification reaction using primer sets 7 and 8 of FIG. 6;

FIGS. 19 A and B are photographs of agarose gels demonstrating the detection of H5N1 avian influenza A virus by one-step RT-PCR; A: amplification of serially diluted in vitro-transcribed single-stranded RNA; B: Specific detection of H5N1 avian influenza virus from field samples;

FIGS. 20 A and B are photographs of agarose gels of PCR products obtained using either A: a two-step RT-PCR reaction; or B: a one-step RT-PCR reaction;

FIG. 20C depicts the results of real time PCR using primer set 6;

FIGS. 21 A, B and C are photographs of agarose gels demonstrating the use of exemplary primers of the invention on field samples to detect H5N1 avian influenza virus; A: samples of allantoic fluid; B: samples of homogenized tissues; and C: comparison of an in-house H5 primer set with an H5N1 primer set;

FIGS. 22 A, B and C depict the results of real time PCR with SYBR green dye using exemplary primers (set 9) directed against the NA gene of H5N1 influenza A; A is an amplification curve obtained during the real time PCR amplification reaction; B are melting curves obtained at the end of the amplification reaction and C is a photograph of a 1.5% agarose gel displaying the PCR amplification products;

FIGS. 23 A, B and C depict the results of real time PCR with SYBR green dye using exemplary primers (set 10) directed against the NA gene of H5N1 influenza A; A is an amplification curve obtained during the real time PCR amplification reaction; B are melting curves obtained at the end of the amplification reaction and C is a photograph of a 1.5% agarose gel displaying the PCR amplification products;

FIGS. 24 A, B and C depict the results of real time PCR with SYBR green dye using exemplary primers (set 11) directed against the NA gene of H5N1 influenza A; A is an amplification curve obtained during the real time PCR amplification reaction; B are melting curves obtained at the end of the amplification reaction and C is a photograph of a 1.5% agarose gel displaying the PCR amplification products;

FIGS. 25 A, B and C depict the results of real time PCR with SYBR green dye using exemplary primers (set 12) directed against the NA gene of H5N1 influenza A; A is an amplification curve obtained during the real time PCR amplification reaction; B are melting curves obtained at the end of the amplification reaction and C is a photograph of a 1.5% agarose gel displaying the PCR amplification products;

FIGS. 26 A, B and C depict the results of real time PCR with SYBR green dye using exemplary primers (set 13) directed against the NA gene of H5N1 influenza A; A is an amplification curve obtained during the real time PCR amplification reaction; B are melting curves obtained at the end of the amplification reaction and C is a photograph of a 1.5% agarose gel displaying the PCR amplification products;

FIGS. 27 A, B and C depict the results of real time PCR with SYBR green dye using exemplary primers (set 14) directed against the NA gene of H5N1 influenza A; A is an amplification curve obtained during the real time PCR amplification reaction; B are melting curves obtained at the end of the amplification reaction and C is a photograph of a 1.5% agarose gel displaying the PCR amplification products;

FIGS. 28 A, B and C depict the results of real time PCR with SYBR green dye using exemplary primers (set 15) directed against the NA gene of H5N1 influenza A; A is an amplification curve obtained during the real time PCR amplification reaction; B are melting curves obtained at the end of the amplification reaction and C is a photograph of a 1.5% agarose gel displaying the PCR amplification products;

FIGS. 29 A, B and C depict the results of real time PCR with SYBR green dye using exemplary primers (set 16) directed against the NA gene of H5N1 influenza A; A is an amplification curve obtained during the real time PCR amplification reaction; B are melting curves obtained at the end of the amplification reaction and C is a photograph of a 1.5% agarose gel displaying the PCR amplification products;

FIGS. 30 A, B and C depict the results of real time PCR with SYBR green dye using exemplary primers (set 17) directed against the NA gene of H5N1 influenza A; A is an amplification curve obtained during the real time PCR amplification reaction; B are melting curves obtained at the end of the amplification reaction and C is a photograph of a 1.5% agarose gel displaying the PCR amplification products;

FIGS. 31A, B and C depict the results of real time PCR with SYBR green dye using exemplary primers (set 18) directed against the NA gene of H5N1 influenza A; A is an amplification curve obtained during the real time PCR amplification reaction; B are melting curves obtained at the end of the amplification reaction and C is a photograph of a 1.5% agarose gel displaying the PCR amplification products;

FIGS. 32 A, B and C depict the results of real time PCR with SYBR green dye using exemplary primers (set 19) directed against the NA gene of H5N1 influenza A; A is an amplification curve obtained during the real time PCR amplification reaction; B are melting curves obtained at the end of the amplification reaction and C is a photograph of a 1.5% agarose gel displaying the PCR amplification products;

FIGS. 33 A, B and C depict the results of real time PCR with SYBR green dye using exemplary primers (set 20) directed against the NA gene of H5N1 influenza A; A is an amplification curve obtained during the real time PCR amplification reaction; B are melting curves obtained at the end of the amplification reaction and C is a photograph of a 1.5% agarose gel displaying the PCR amplification products;

FIGS. 34 A B, C and D depict the results of real time PCR with SYBR green dye using exemplary primers (set 21) directed against the HA gene of H5N1 influenza A; A is an amplification curve obtained during the real time PCR amplification reaction; B is an RNA standard curve; C are melting curves obtained at the end of the amplification reaction and D is a photograph of a 1.5% agarose gel displaying the PCR amplification products;

FIGS. 35 A B, C and D depict the results of real time PCR with SYBR green dye using exemplary primers (set 22) directed against the HA gene of H5N1 influenza A; A is an amplification curve obtained during the real time PCR amplification reaction; B is an RNA standard curve; C are melting curves obtained at the end of the amplification reaction and D is a photograph of a 1.5% agarose gel displaying the PCR amplification products;

FIGS. 36 A, B, C and D depict the results of real time PCR with SYBR green dye using exemplary primers (set 23) directed against the HA gene of H5 influenza A (H5N1 (QS 1 to QS 5), H5N2 (a) and H5N3 (b)); A is an amplification curve obtained during the real time PCR amplification reaction; B is an RNA standard curve; C are melting curves obtained at the end of the amplification reaction and D is a photograph of a 1.5% agarose gel displaying the PCR amplification products;

FIG. 37 is a photograph of an agarose gel displaying the relative amounts of amplification product obtained by a one-step RT-PCR method, using varying amounts of template and primer set 10;

FIG. 38 is a photograph of an agarose gel displaying the relative amounts of amplification product obtained by a one-step RT-PCR method, using varying amounts of template and primer set 11;

FIG. 39 is a photograph of an agarose gel displaying the relative amounts of amplification product obtained by a one-step RT-PCR method, using varying amounts of template and primer set 13;

FIG. 40 is a photograph of an agarose gel displaying the relative amounts of amplification product obtained by a one-step RT-PCR method, using varying amounts of template and primer set 16;

FIG. 41 is a photograph of an agarose gel displaying the relative amounts of amplification product obtained by a two-step RT-PCR method, using varying amounts of template and primer set 12;

FIG. 42 is a photograph of an agarose gel displaying the relative amounts of amplification product obtained by a two-step RT-PCR method, using varying amounts of template and primer set 15;

FIGS. 43 A and B are photographs of agarose gels displaying the relative amounts of amplification product using varying amounts of template and primer set 23, obtained by A: a one-step RT-PCR method; and B: a two-step RT-PCR method;

FIG. 44 is an amplification curve obtained using the Taqman™ real time PCR method and primer set 24, directed against the HA gene of subtype H5;

FIG. 45 is an amplification curve obtained using the Taqman™ real time PCR method and primer set 25, directed against the HA gene of subtype H5N1;

FIG. 46 is an amplification curve obtained using the Taqman™ real time PCR method and primer set 26, directed against the HA gene of subtype H5N1;

FIG. 47 is a Delta Rn curve obtained using the Taqman™ real time RT-PCR method and primer set 25, directed against the HA gene of subtype H5N1;

FIG. 48 is a Delta Rn curve obtained using the Taqman™ real time RT-PCR method and primer set 26, directed against the HA gene of subtype H5N1;

FIG. 49 is a Delta Rn curve obtained using the Taqman™ real time RT-PCR method and primer set 27, directed against the NA gene of subtype H5N1;

FIG. 50 is a Delta Rn curve obtained using the Taqman™ real time RT-PCR method and primer set 27, directed against the NA gene of subtype H5N1; and

FIG. 51 is a Delta Rn curve obtained using the Taqman™ real time RT-PCR multiplex method and primer sets 25 and 27, directed against the HA and NA genes of subtype H5N1.

### DETAILED DESCRIPTION

RNA viruses, including the influenza A virus, tend to have high mutation rates due to the low fidelity nature of RNA replication when compared to DNA replication. As a result, influenza viruses tend to evolve rapidly. Furthermore, influenza A viruses tend to undergo genetic reassortment between viral strains, which mechanism has contributed to the development of the various HA and NA subtypes. The inventors compared the sequence of the hemagglutinin (“HA”) gene from more than 200 influenza A H5 isolates, and more than 100 influenza A H5N1 isolates. As well, the inventors compared the sequence of the neuraminidase (“NA”) gene from approximately 70 influenza A H5N1 isolates. Surprisingly, despite the high mutation rate within influenza viruses, the inventors have discovered short regions of highly conserved sequences unique to specific subtypes, which regions are suitable to identify or detect the presence of those subtypes in a sample.

The sequences used in the comparison were obtained from publicly available databases and were compared using a variety of sequence comparison software, including the software ClustalW.

These sequence comparisons allowed the inventors to develop forward and reverse primers set out in SEQ ID NO:1 to SEQ ID NO:114, directed to conserved regions of the HA gene or the NA gene of avian influenza virus subtypes H5 or H5N1, for use in a detection assay, for example, reverse-transcription followed by polymerase chain reaction amplification (“RT-PCR”). The comparison of such a large number of viral isolates allowed for the design of primers directed to well-conserved regions of the HA or NA gene, thus targeting regions that are less likely to be affected by mutational changes and thereby providing primers that can detect a larger pool of H5 or H5N1 variants than primers that are currently available.

The term “isolate” as used herein refers to a particular virus or clonal population of virus particles, isolated from a particular biological source, such as a patient, which has a particular genetic sequence. Different isolates may vary at only one or several nucleotides, and may still fall within the same viral subtype. A viral subtype refers to any of the subtypes of HA or subtypes of NA classified according to the antigenicity of these glycoproteins.

The inventors found that in certain conserved regions, one or more nucleotides at a specific location varied between isolates. For those regions, a family of primers has been developed, each primer within the family being based on a conserved sequence of the HA or the NA gene, but varying at one or more particular bases within the conserved sequence.

Thus, in one aspect the invention provides a primer comprising a sequence as set out in any one of SEQ ID NO:1 to SEQ ID NO:114.

As will be understood by a skilled person, a “primer” is a single-stranded DNA or RNA molecule of defined sequence that can base pair to a second DNA or RNA molecule that contains a complementary sequence (the target). The stability of the resulting hybrid molecule depends upon the extent of the base pairing that occurs, and is affected by parameters such as the degree of complementarity between the primer and target molecule and the degree of stringency of the hybridization conditions. The degree of hybridization stringency is affected by parameters such as the temperature, salt concentration, and concentration of organic molecules, such as formamide, and may be determined using methods that are known to those skilled in the art. Primers can be used for methods involving nucleic acid hybridization, such as nucleic acid sequencing, nucleic acid amplification by the polymerase chain reaction, single stranded conformational polymorphism (SSCP) analysis, restriction fragment polymorphism (RFLP) analysis, Southern hybridization, northern hybridization, in situ hybridization, electrophoretic mobility shift assay (EMSA), nucleic acid microarrays, and other methods that are known to those skilled in the art.

The term “RNA” refers to a sequence of two or more covalently bonded, naturally occurring or modified ribonucleotides. The RNA may be single stranded or double stranded. The term “DNA” refers to a sequence of two or more covalently bonded, naturally occurring or modified deoxyribonucleotides, including cDNA and synthetic (e.g., chemically synthesized) DNA, and may be double stranded or single stranded. By “reverse transcribed DNA” or “DNA reverse transcribed from” is meant complementary or copy DNA (cDNA) produced from an RNA template by the action of RNA-dependent DNA polymerase (reverse transcriptase).

Avian influenza virus is a single stranded RNA virus and in some embodiments, the primer has a DNA sequence that corresponds to the RNA sequence of a conserved region of the HA gene of avian influenza virus subtype H5 or H5N1 (SEQ ID NO:1 to SEQ ID NO:31 and SEQ ID NO:112), as set out in Table 1. Such primers may be used as a forward primer when sequencing or amplifying DNA reverse transcribed from the HA gene of subtypes H5 or H5N1.

TABLE 1

Forward Primers for the HA
Gene of Subtype H5 or H5N1

| | | | |
|---|---|---|---|
| SEQ ID | | Start | Sequence |
| NO: | Primer | Nucleotide | (5′ to 3′) |

| | | | |
|---|---|---|---|
| 1 | gisAFH5H1aF | 84 | ATT TGC ATT GGT |
| | | | TAC CAT GCA AA |
| 2 | gisAFH5H1bF | 84 | ATT TGC ATT GGT |
| | | | TAT CAT GCA AA |
| 3 | gisAFH5H1cF | 84 | ATC TGC ATT GGT |
| | | | TAT CAT GCA AA |
| 4 | gisAFH5H1dF | 84 | ATC TGC ATC GGT |
| | | | TAT CAT GCA AA |
| 5 | gisAFH5H1eF | 84 | ATT TGC ATC GGT |
| | | | TAC CAT GCA AA |
| 6 | gisAFH5H1fF | 84 | ATT TGC ATT GGT |
| | | | CAT CAT GCA AA |
| 7 | gisAFH5H3aF | 248 | GCT GGA TGG CTC |
| | | | CTC GGA AAC CC |
| 8 | gisAFH5H3bF | 248 | GCT GGA TGG CTC |
| | | | CTT GGA AAT CC |
| 9 | gisAFH5H3cF | 248 | GCT GGA TGG CTC |
| | | | CTC GGA AAT CC |
| 10 | gisAFH5H3dF | 248 | GCT GGA TGG CTT |
| | | | CTT GGG AAC CC |
| 11 | gisAFH5H3eF | 248 | GCT GGA TGG CTC |
| | | | CTG GGA AAC CC |
| 12 | gisAFH5H3fF | 248 | GCT GGA TGG CTT |
| | | | CTT GGA AAC CC |
| 13 | gisAFH5H3gF | 248 | GCC GGA TGG CTT |
| | | | TTG GGG AAT CC |
| 14 | gisAFH5H3hF | 248 | GCT GGG TGG CTT |
| | | | CTT GGA AAC CC |
| 15 | gisAFH5H3iF | 248 | GCT GGG TGG CTT |
| | | | CTT GGA AAT CC |
| 16 | gisAFH5H3jF | 248 | GCT GGA TGG CTT |
| | | | CTC GGA AAT CC |
| 17 | gisAFH5H3kF | 248 | GCT GGG TGG CTC |
| | | | CTC GGA AAC CC |
| 18 | gisAFH5H31F | 248 | GCC GGA CTT GTT |
| | | | TTG GGG AAT CC |
| 19 | gisAFH5N1H1F | 81 | CAG ATT TGC ATT |
| | | | GGT TAC CAT GC |
| 20 | gisAFH5N1H2aF | 122 | GTT GAC ACA ATA |
| | | | ATG GAA AAG AA |
| 21 | gisAFH5N1H2bF | 122 | GTT GAT ACA ATA |
| | | | ATG GAA AAG AA |
| 22 | gisAFH5N1H2cF | 122 | GTT GAC ACA ATA |
| | | | ATG GAG AAG AA |
| 23 | gisAFH5N1H3F | 248 | GCT GGA TGG CTC |
| | | | CTC GGA AAC CC |
| 24 | gisAFH5N1H6aF | 366 | ACT ATG AAG AAC |
| | | | TGA AAC ACC T |
| 25 | gisAFH5N1H6bF | 366 | ACT ATG AAG AAT |
| | | | TGA AAC ACC T |
| 26 | gisAFH5N1H7aF | 800 | GAG AGT AAT GGA |
| | | | AAT TTC ATT GC |
| 27 | gisAFH5N1H7bF | 800 | GAG AGT AAT GGA |
| | | | AAT TTT ATT GC |
| 28 | gisAFH5N1H9F | 830 | TGC ATA CAA AAT |
| | | | TGT CAA GAA AGG |
| 29 | gisAFH5N1H12aF | 941 | ATG CCA T/CTC CAC |
| | | | AAC/T ATA CAC CC |
| 30 | gisAFH5N1H12bF | 941 | ATG CCA TTC CAC |
| | | | AAT ATA CAC CC |
| 31 | gisAFH5N1H13F | 1439 | CAG CTT AGG GAT |
| | | | AAT GCA AAG G |
| 112 | gisAFH5N1HTqF1 | 827 | GGG ACT CAA CAA |
| | | | TTA TGA AAA GTG |
| | | | AA |

In some embodiments, the primer has a DNA sequence that corresponds to the RNA sequence of a conserved region of the HA gene of avian influenza virus subtype H5 or H5N1 (SEQ ID NO:32 to SEQ ID NO:71, SEQ ID NO:113 and SEQ ID NO:114), as set out in Table 2. Such primers may be used as a reverse primer when sequencing or amplifying a first strand DNA reversed transcribed from the HA gene of subtypes H5 or H5N1.

TABLE 2

Reverse Primers for the HA
Gene of Subtype H5 or H5N1

| | | | |
|---|---|---|---|
| SEQ ID | | Start | Sequence |
| NO: | Primer | Nucleotide | (5′ to 3′) |

| | | | |
|---|---|---|---|
| 32 | gisAFH5H2aR | 270 | GGG TTT CCG AGG |
| | | | AGC CAT CCA GC |
| 33 | gisAFH5H2bR | 270 | GGA TTT CCA AGG |
| | | | AGC CAT CCA GC |
| 34 | gisAFH5H2cR | 270 | GGA TTT CCG AGG |
| | | | AGC CAT CCA GC |
| 35 | gisAFH5H2dR | 270 | GGG TTC CCA AGA |
| | | | AGC CAT CCA GC |
| 36 | gisAFH5H2eR | 270 | GGG TTT CCC AGA |
| | | | AGC CAT CCA GC |
| 37 | gisAFH5H2fR | 270 | GGG TTT CCA AGA |
| | | | AGC CAT CCA GC |
| 38 | gisAFH5H2gR | 270 | GGA TTC CCC AAA |
| | | | AGC CAT CCG GC |
| 39 | gisAFH5H2hR | 270 | GGG TTT CCA AGA |
| | | | AGC CAC CCA GC |
| 40 | gisAFH5H2iR | 270 | GGA TTT CCA AGA |
| | | | AGC CAC CCA GC |
| 41 | gisAFH5H2jR | 270 | GGA TTT CCG AGA |
| | | | AGC CAT CCA GC |
| 42 | gisAFH5H2kR | 270 | GGG TTT CCG AGG |
| | | | AGC CAC CCA GC |
| 43 | gisAFH5H21R | 270 | GGA TTC CCC AAA |
| | | | ACA AGT CCG GC |
| 44 | gisAFH5H4aR | 966 | GGG TGT ATG TTG |
| | | | TGG AAT GGC AT |
| 45 | gisAFH5H4bR | 966 | GGG TGT ATA TTG |
| | | | TGG AAG GGC AT |
| 46 | gisAFH5H4cR | 966 | GGA TGT ATA TTG |
| | | | TGG AAT GGC AT |
| 47 | gisAFH5H4dR | 966 | GGA TGA ACA TTG |
| | | | TGG AAA GGC AT |
| 48 | gisAFH5H4eR | 966 | GGA TGG ACA TTA |
| | | | TGG AAA GGC AT |
| 49 | gisAFH5H4fR | 966 | GGG TGT ATG TTA |
| | | | TGG AAT GGC AT |
| 50 | gisAFH5H4gR | 966 | GGA TGT ACA TTG |
| | | | TGA AAA GGC AT |
| 51 | gisAFH5H4hR | 966 | GGA TGA ACA TTG |
| | | | TGG AAG GGC AT |
| 52 | gisAFH5H4iR | 966 | GGA TGT ATA TTG |
| | | | TGG AAC GGC AT |
| 53 | gisAFHSH4jR | 966 | GGA TGT ATG TTG |
| | | | TGG AAT GGC AT |
| 54 | gisAFH5H4kR | 966 | GGA TGT ATG TTG |
| | | | TGG AAC GGC AT |
| 55 | gisAFH5H41R | 966 | GGG TGT ATA TTG |
| | | | TGG AAT GGC AT |
| 56 | gisAFH5N1H4R | 270 | GGG TTT CCG AGG |
| | | | AGC CAT CCA GC |
| 57 | gisAFH5N1H5aR | 387 | AGG TGT TTC AGT |
| | | | TCT TCA TAG T |
| 58 | gisAFHSN1H5bR | 387 | AGG TGT TTC AAT |
| | | | TCT TCA TAG T |
| 59 | gisAFH5N1H8aR | 822 | GCA ATG AAA TTT |
| | | | CCA TTA CTC TC |
| 60 | gisAFH5N1H8bR | 822 | GCA ATA AAA TTT |
| | | | CCA TTA CTC TC |
| 61 | gisAFH5N1H10R | 855 | CCT TTC TTG ACA |
| | | | ATT TTG TAT GCA |
| 62 | gisAFH5N1H11aR | 963 | GGG TGT ATG TTG |
| | | | TGG AAT GGC AT |
| 63 | gisAFH5N1H11bR | 963 | GGG TGT ATA TTG |
| | | | TGG AGT GGC AT |
| 64 | gisAFH5N1H11cR | 963 | GGG TGT ATA TTG |
| | | | TGG AAT GGC AT |
| 65 | gisAFH5N1H14R | 1460 | CCT TTG CAT TAT |
| | | | CCC TAA GCT G |
| 66 | gisAFH5N1H15aR | 1602 | TCC AAT TTT ACT |
| | | | CCA CTT ATT TC |
| 67 | gisAFH5N1H15bR | 1602 | TCC AAT TTT ACC |
| | | | CCA CTT ATT TC |
| 68 | gisAFH5N1H15cR | 1602 | TCC AAT TTT ACT |
| | | | CCA CTT ATT TT |
| 69 | gisAFH5N1H16aR | 1657 | TAG GGA ACT CGC |
| | | | CAC TGT TGA AT |
| 70 | gisAFH5N1H16bR | 1657 | TAG GGA ACT CGC |
| | | | CAC TGC TGA AT |
| 71 | gisAFH5N1H16cR | 1657 | TAG GGA ACT CGC |
| | | | CAC TGT AGA AT |
| 113 | gisAFH5HTqR | 111 | GGC ATG TGT AAC |
| | | | AGT AAC GTT CTT T |
| 114 | gisAFH5N1HTqR1 | 871 | CAT TGG AGT TTG |
| | | | ACA CTT GGT GTT |

In some embodiments, the primer has a DNA sequence that corresponds to the RNA sequence of a conserved region of the NA gene of avian influenza virus subtype H5N1, as set out in SEQ ID NO:72 to SEQ ID NO:93 (see Table 3). Such primers may be used as a forward primer when sequencing or amplifying DNA reversed transcribed from the NA gene of subtype H5N1.

TABLE 3
Forward Primers for the NA Gene of Subtype H5N1

| | | | |
|---|---|---|---|
| SEQ ID | | Start | Sequence |
| NO: | Primer | Nucleotide | (5′ to 3′) |

| | | | |
|---|---|---|---|
| 72 | gisAFHSN1N1aF | 540 | TTT GAG TCT GTT |
| | | | GCT TGG TCA GC |
| 73 | gisAFH5N1N1bF | 540 | TTT GAG TCG GTT |
| | | | GCT TGG TCG GC |
| 74 | gisAFHSN1N1cF | 540 | TTT GAG TCT GTT |
| | | | GCT TGG TCG GC |
| 75 | gisAFH5N1N2aF | 549 | GTT GCT TGG TCA |
| | | | GCA AGT GCT TG |
| 76 | gisAFH5N1N2bF | 549 | GTT GCT TGG TCG |
| | | | GCA AGT GCT TG |
| 77 | gisAFHSN1N2cF | 549 | GTT GCT TGG TCG |
| | | | GCA AGC GCT TG |
| 78 | gisAFH5N1N4aF | 638 | GAA ATA CAA TGG |
| | | | CAT AAT AAC AG |
| 79 | gisAFH5N1N4bF | 638 | GAA ATA CAA CGG |
| | | | CAT AAT AAC AG |
| 80 | gisAFH5N1N4cF | 638 | GAA ATA CAA TGG |
| | | | CAT GAT AAC AG |
| 81 | gisAFHSN1N5aF | 834 | CCT AAT TAT CAT |
| | | | TAC GAG GAA T |
| 82 | gisAFH5N1N5bF | 834 | CCT AAT TAT CAC |
| | | | TAT GAG GAG T |
| 83 | gisAFH5N1N5cF | 834 | CCT AAT TAC CAC |
| | | | TAT GAG GAG T |
| 84 | gisAFHSNlN5dF | 834 | CCT AAT TAC CAC |
| | | | TAT GAG GAA T |
| 85 | gisAFH5N1N5eF | 834 | CCT AAT TAT CAT |
| | | | TAC CAG GAA T |
| 86 | gisAFH5N1N5fF | 834 | CCT AAT TAT CAT |
| | | | TAC GAA GAA T |
| 87 | gisAFH5N1N5gF | 834 | CCT AAT TAT CAC |
| | | | TAT GAG GAA T |
| 88 | gisAFHSN1N8aF | 963 | GGA TAT ATA TGC |
| | | | AGT GGG GTT |
| 89 | gisAFH5N1N8bF | 963 | GGA TAT ATA TGC |
| | | | AGT GGA GTT |
| 90 | gisAFHSN1N8cF | 963 | GGA TAC ATA TGC |
| | | | AGT GGG GTT |
| 91 | gisAFH5N1N10aF | 1062 | GGG TTT TCA TTT |
| | | | AAA TAC GGC |
| 92 | gisAFH5N1N10bF | 1062 | GGG TTT TCA TTT |
| | | | AAG TAT GGC |
| 93 | gisAFH5N1N10cF | 1062 | GGG TTT TCA TTT |
| | | | AAA TAT GGC |

In some embodiments, the primer has a DNA sequence that corresponds to the RNA sequence of a conserved region of the NA gene of avian influenza virus subtype H5N1, as set out in SEQ ID NO:94 to SEQ ID NO:111 and SEQ ID NO:118 (see Table 4). Such primers may be used as a reverse primer when sequencing or amplifying DNA reversed transcribed from the NA gene of subtype H5N1.

TABLE 4
Reverse Primers for the NA Gene of Subtype H5N1

| | | | |
|---|---|---|---|
| SEQ ID | | Start | Sequence |
| NO: | Primer | Nucleotide | (5′ to 3′) |

| | | | |
|---|---|---|---|
| 94 | gisAFH5N1N3aR | 660 | CTG TTA TTA TGC |
| | | | CAT TGT ATT TC |
| 95 | gisAFH5N1N3bR | 660 | CTG TTA TCA TGC |
| | | | CAT TGT ATT TC |
| 96 | gisAFH5N1N3cR | 660 | CTG TTA TTA TGC |
| | | | CGT TGT ATT TC |
| 97 | gisAFH5N1N6aR | 855 | ATT CCT CGT AAT |
| | | | GAT AAT TAG G |
| 98 | gisAFH5N1N6bR | 855 | ACT CCT CAT AGT |
| | | | GAT AAT TAG G |
| 99 | gisAFH5N1N6cR | 855 | ACT CCT CAT AGT |
| | | | GGT AAT TAG G |
| 100 | gisAFH5N1N6dR | 855 | ATT CCT CAT AGT |
| | | | GGT AAT TAG G |
| 101 | gisAFHSN1N6eR | 855 | ATT CCT GGT AAT |
| | | | GAT AAT TAG G |
| 102 | gisAFH5N1N6fR | 855 | ATT CTT CGT AAT |
| | | | GAT AAT TAG G |
| 103 | gisAFH5N1N6gR | 855 | ATT CCT CAT AGT |
| | | | GAT AAT TAG G |
| 104 | gisAFH5N1N7aR | 992 | GTC TCC GAA AAC |
| | | | CCC ACT GCA |
| 105 | gisAFH5N1N7bR | 992 | GTC TCC GAA AAC |
| | | | TCC ACT GCA |
| 106 | gisAFH5N1N9aR | 1082 | GCC GTA TTT AAA |
| | | | TGA AAA CCC |
| 107 | gisAFH5N1N9bR | 1082 | GCC ATA CTT AAA |
| | | | TGA AAA CCC |
| 108 | gisAFHSN1N9cR | 1082 | GCC ATA TTT AAA |
| | | | TGA AAA CCC |
| 109 | gisAFH5N1N11aR | 1234 | CTC CCG CTG/A |
| | | | TAT/G CCT GAC CAA |
| | | | TCAG |
| 110 | gisAFH5N1N11bR | 1234 | CTC CCG CTA TAT |
| | | | CCT GAC CAA TCA G |
| 111 | gisAFH5N1N11cR | 1234 | CTC CCG CTA TAG |
| | | | CCT GAC CAA TCA |
| | | | G |
| 118 | gisAFH5N1NTqR1 | 95 | TCT GGG CCA GAA |
| | | | ATT CCA AT |

Where a nucleotide in a particular position varied within the conserved region of the HA or the NA gene for the viral isolates tested, a “family” of primers was developed based on the conserved region of the gene, in which one or more residue within the family of primers varied from primer to primer. For example, SEQ ID NO: 1 to SEQ ID NO:6 are such a family.

Furthermore, a skilled person will understand that, although the primers are based on conserved sequences, one or more bases within the conserved sequences can be substituted, inserted or deleted, provided that the mutated primer will still hybridize with the target sequence in a sample with the same or similar stringency as the original primer sequence. Hybridization conditions may be modified in accordance with known methods depending on the sequence of interest (see Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, N.Y.). Generally, stringent conditions are selected to be about 5° C. lower than the thermal melting point for the specific sequence at a defined ionic strength and pH.

A skilled person will understand that having multiple substitution mutations in a short sequence will decrease the strength of hybridization of the primer to the complement of the original, unmutated primer, and that the spacing and location of the mutations within the primer sequence will also affect the strength or stringency of hybridization. Furthermore, a skilled person will understand that insertion or deletion of one or more nucleotides in a short sequence will also decrease the strength of hybridization of the primer to the complement of the original, unmutated primer, and that having insertions or deletions of one or more nucleotides in more than one location in a short sequence may significantly alter the hybridization of the primer to the complement of the unmutated sequence.

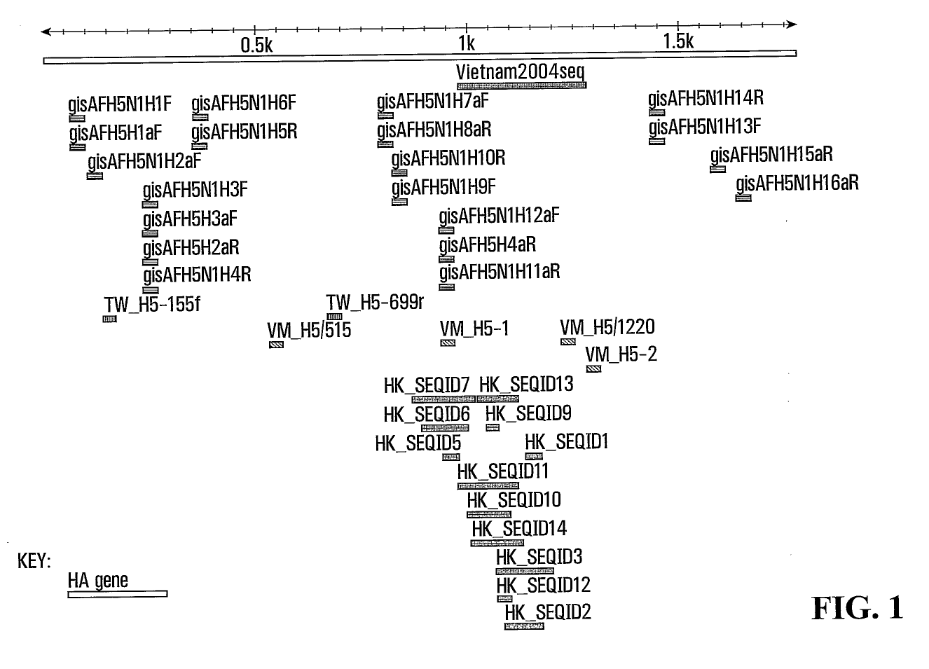

The alignment of the various primers of the invention for the HA gene of avian influenza virus are set out in FIG. 1, along with other primers previously known to be directed against the HA gene of subtype 5. Primers TW_H5-155f and TW_H5-699r were published in Lee et al. (2001, J. Viriol. Methods 97:13-22). Primers VM_H5/515, VM_H5-1, VM_H5/1220 and VM_H5-2 were published in Hien et al. (2004, New Eng. J of Med. 350(12):1179-1188). Primers HK_SEQ ID1 to HK_SEQ ID3, HK_SEQ ID5 to HK_SEQ ID7 and HK_SEQ ID9 to HK_SEQ ID14 were published in WO 02/29118.

In some embodiments, the primer may be modified with a label to allow for detection of the primer or a DNA product synthesized or extended from the primer. For example, the label may be a fluorescent label, a chemiluminescent label, a coloured dye label, a radioactive label, a radiopaque label, a protein including an enzyme, a peptide or a ligand for example biotin.

In certain embodiments, the primer may comprise an additional nucleotide sequence in addition to a sequence of any one of SEQ ID NO:1 to SEQ ID NO:114. Such an additional sequence may be encoded by or complementary to the sequence of the HA or NA gene flanking the sequence defined by any one of SEQ ID NO:1 to SEQ ID NO:114, with the proviso that the term primer as used herein is not the entire influenza A genome and is not primer TW_H5-155f, primer TW_H5-699r, primer VM_H5/515, primer VM_H5-1, primer VM_H5/1220, primer VM_H5-2, or any of HK_SEQ ID1 to HK_SEQ ID3, HK_SEQ ID5 to HK_SEQ ID7 and HK_SEQ ID9 to HK_SEQ ID14, described above.

Alternatively, the additional sequence may not be directed to the HA or NA gene, but may be a sequence, for example, that is recognised by a protein or an enzyme, for example a restriction enzyme, or that is complementary to a nucleic acid sequence that is used for detection, for example, that is complementary to a probe that may be labelled. A skilled person will understand that there will be an optimum length and sequence for the primer, depending on the application for which the primer is to be used, so as to suitably limit the number and type of any such additional sequences. For example, a PCR primer should not be of such length or sequence that the temperature above which it no longer specifically binds to the template approaches the temperature at which the extension by polymerase occurs.

Therefore, in certain embodiments, the primer consists essentially of the sequence of any one of SEQ ID NO:1 to SEQ ID NO:114, meaning the primer may include one or more additional nucleotides, 5′ to, 3′ to, or flanking on either side, of the sequence of any one of SEQ ID NO:1 to SEQ ID NO:114, but that the additional nucleotides should not significantly affect the hybridization of the sequence of any one of SEQ ID NO:1 to SEQ ID NO:114 to a nucleic acid molecule containing the complementary sequence. For example, the addition of several nucleotides on either side of a short primer sequence should not alter the hybridization stringency of the short primer sequence to its complementary sequence even when contained within a larger sequence, to such an extent that the short primer sequence cannot hybridize with the same or similar stringency as when the additional nucleotides are not present. That is, since the regions in the influenza HA or NA gene surrounding the sequences described herein may vary among isolates, a primer consisting essentially of the sequence of any one of SEQ ID NO:1 to SEQ ID NO:114 should not include so much of the viral sequences flanking the conserved sequences described herein so as to affect the sensitivity and ability to detect a wide range of H5 or H5N1 isolates. In certain other embodiments the primer consists of, or is, the sequence of any one of SEQ ID NO:1 to SEQ ID NO:114.

In certain embodiments, the primer comprises a “target annealing sequence” which comprises a sequence of any one of SEQ ID NO:1 to SEQ ID NO:114, and a non-influenza virus A sequence.

The target annealing sequence will hybridize to at least a portion of a target nucleic acid in a sample, the target nucleic acid being homologous to, complementary to, transcribed or reverse transcribed from, or otherwise derived from, an influenza A H5 or H5N1 viral subtype. Thus, the target annealing sequence may also include flanking sequences encoded by or complementary to the sequence of the HA or NA gene flanking the sequence defined by any one of SEQ ID NO:1 to SEQ ID NO:114. The target annealing sequence may alternatively consist essentially of, or consist of, a sequence of SEQ ID NO:1 to SEQ ID NO:114.

The non-influenza A virus sequence is a sequence that is not derived from or corresponding or complementary to the influenza A viral genome sequence. As described above, the non-influenza A virus sequence may be a sequence, for example, that is recognised by a protein or an enzyme, for example a restriction enzyme, or that is complementary to a nucleic acid sequence that is used for detection, for example, that is complementary to a probe that may be labelled or to a capture sequence of an immobilized nucleic acid molecule that may be used to capture the present primer. The non-influenza A virus sequences may be located 5′ to, 3′ to, or may flank on either side, the target annealing sequence.

The length of the primer or primers of the invention will depend on the desired use or application. For example, as will be understood, a PCR primer will typically be between about 15 and about 35 bases in length. The length of a PCR primer will be based on the sequence that is to be amplified as well as the desired melting temperature of the primer/template hybrid. However, for applications such as Southern hybridizations, the primer may be longer, for example from about 15 bases to about 1 kilobase in length or longer. Thus, the primer may be from 15 bases to about 1 kilobase in length, from 15 to about 500 bases, from 15 to about 300 bases, from 15 to about 150 bases, from 15 to about 100 bases or from 15 to 50 about bases.

The primers of the invention may be prepared using conventional methods known in the art. For example, standard phosphoramidite chemical ligation methods may be used to synthesize the primer in the 3′ to 5′ direction on a solid support, including using an automated nucleic acid synthesizer. Such methods will be known to a skilled person.

Although the term “primer” is used herein to describe single-stranded nucleotides that are used to anneal in a sequence-specific manner to a template sequence and initiate a new strand synthesis, a skilled person will understand that uses of the primers of the invention are not so limited. For example, the primers of the invention may be used as probes, to detect a complementary sequence to which the probe hybridizes. For such a use, the primer will typically be labelled for detection, for example, with a fluorescent label, a chemiluminescent label, a coloured dye label, a radioactive label, a protein including an enzyme, a peptide or a ligand for example biotin. When used as probes, the primers may be used in nucleic acid hybridization methods, single stranded conformational polymorphism (SSCP) analysis, restriction fragment polymorphism (RFLP) analysis, Southern hybridization, northern hybridization, in situ hybridization, electrophoretic mobility shift assay (EMSA), nucleic acid microarrays, and other methods that are known to those skilled in the art.

The primers of the invention may be used to diagnose or detect avian influenza subtype H5 or H5N1 in a sample, for example a biological sample derived from an organism suspected of carrying the virus.

Thus, there is provided a method for detecting avian influenza subtype H5 in a sample comprising amplifying DNA reverse transcribed from RNA obtained from the sample using one or more reverse primers comprising any one of the sequences of SEQ ID NO:32 to SEQ ID NO:55 and one or more forward primers comprising any one of the sequences of SEQ ID NO:1 to SEQ ID NO:18, and detecting a product of amplification, wherein the product indicates the presence of an avian influenza virus H5 subtype in the sample. There is also provided a method for detecting avian influenza subtype H5N1 in a sample comprising amplifying DNA reverse transcribed from RNA obtained from the sample using one or more reverse primers comprising any one of the sequences of SEQ ID NO:56 to SEQ ID NO:71, SEQ ID NO: 113 and SEQ ID NO: 114 and one or more forward primers comprising any one of the sequences of SEQ ID NO:19 to SEQ ID NO:31 and SEQ ID NO:112, or using one or more reverse primers comprising any one of the sequences of SEQ ID NO:94 to SEQ ID NO:111 and SEQ ID NO:118 and one or more forward primers comprising any one of the sequences of SEQ ID NO:72 to SEQ ID NO:93, and detecting a product of amplification, wherein the product indicates the presence of an avian influenza virus H5N1 subtype in the sample.

The term “detecting” an amplification product is intended to include determining the presence or absence, or quantifying the amount, of a product resulting from an amplification reaction that used template, primers, and an appropriate polymerase enzyme.

Typically, RNA from a sample is reverse transcribed so as to provide a single DNA strand that is complementary to the RNA HA gene or to the RNA NA gene. The reverse transcribing is performed using a reverse transcriptase enzyme that is capable of reading an RNA template and synthesizing a complementary DNA strand from a primer that binds to the RNA template, by polymerizing DNA nucleotides in a sequence complementary to that of the RNA template. Reverse transcriptase enzymes, for example T7 reverse transcriptase, are commercially available, and will be known to a skilled person. The reverse transcription reaction is typically performed in a buffer, under reaction conditions and at a temperature that are designed to optimize the reverse transcriptase activity. Commercially supplied reverse transcriptase enzymes may be supplied with a suitable buffer and DNA nucleotides.

The primer used in the reverse transcription reaction may be a mixture of random hexamers that will bind to random sites along the RNA template. Alternatively, the reverse transcription primer may be a specific primer designed to bind at a particular site within the HA gene or the NA gene. Therefore, one or more reverse primers comprising any one of SEQ ID NO:32 to SEQ ID NO:71, SEQ ID NO:113, and SEQ ID NO:114 or SEQ ID NO:94 to SEQ ID NO:111 and SEQ ID NO:118, as set out in Tables 2 and 4, may be used as a primer in the reverse transcription reaction. The same reverse primer or primers of the invention may be advantageously used in the amplification step, particularly when the reverse transcription and amplification are effected in the same reaction. Where more than one primer of the invention is used, each of the primers used will have a different sequence, the sequence of each primer comprising any one of SEQ ID NO:32 to SEQ ID NO:71, SEQ ID NO:113, and SEQ ID NO:114 or SEQ ID NO:94 to SEQ ID NO:111 and SEQ ID NO:118.

Where there is a family of primers based on the same conserved region of the HA gene or NA gene but varying at one or more nucleotides within the primer sequence, for example SEQ ID NO:32 to SEQ ID NO: 43, one or more reverse primers from such a family may be used. This allows for reverse transcription of, and therefore eventual detection of, a wide number of possible isolates or variants of avian influenza virus subtype H5 or H5N1. A “variant” as used herein refers to an H5 subtype in which the HA gene sequence may vary from that of another H5 subtype, or an H5N1 subtype in which the HA gene sequence or the NA gene sequence may vary from that of another H5N1 subtype.

The template RNA for the reverse transcription reaction may be obtained from a sample using RNA extraction methods known in the art. RNA extraction kits are also commercially available, for example, RNeasy kits (Qiagen), and the availability and use of such kits will be known and understood by a skilled person.

The sample may be a biological sample, for example any sample collected from an individual suspected of carrying avian influenza virus subtype H5 or H5N1. The sample may be any sample that contains the virus from an infected individual, and includes tissue and fluid samples, for example, blood, serum, plasma, peripheral blood cells including lymphocytes and mononuclear cells, sputum, mucous, urine, feces, throat swab samples, dermal lesion swab samples, cerebrospinal fluids, pus, and tissue including spleen, kidney and liver.

Once the reverse transcription reaction is completed, the single-stranded DNA molecule can be used in the amplification reaction. The term “amplifying” or “amplification” refers to a reaction in which a nucleic acid molecule that is to be detected so as to indicate the presence of avian influenza virus subtype H5 or H5N1, is reproduced in large quantities. A suitable polymerase enzyme will be used to synthesize a new strand of a template nucleic acid, either RNA or DNA as the case may be, to generate multiple copies.

The amplification step may be performed in the same reaction as the reverse transcription reaction, provided the conditions and reagents from the reverse transcription do not interfere with the amplification reaction. Alternatively, the reverse transcription product may be purified prior to being used as template in the amplification reaction.

Alternatively, a double-stranded DNA molecule, for example a double stranded DNA derived from a reverse transcribed single stranded DNA molecule, may be used as a template for the amplification reaction. If a DNA clone of a particular viral isolate has been made, the DNA clone may be used as a template for amplification. A skilled person will understand how to make a double stranded DNA clone from a viral isolate, using standard techniques. “DNA reverse transcribed from RNA” of a sample is intended to include all such DNA derived from the DNA reverse transcribed from the RNA.

In certain embodiments, amplification is performed by a PCR amplification reaction. Thus, the amplification step may be performed with a DNA polymerase, for example, Taq polymerase, using standard methods and techniques that are known to a person skilled in the art. DNA polymerases for use in amplification of DNA molecules are commercially available. The amplification reaction is performed under conditions and with the necessary reagents, such as deoxynucleotides, buffer and relevant forward and reverse primers, so as to optimize the polymerization activity of the DNA polymerase enzyme.

The PCR amplification reaction involves a denaturation segment, in which the reaction is heated to a temperature sufficient to denature the transcribed DNA strand, and the template RNA if present, and to prevent binding of the primers to either strand. The denaturation segment is followed by an annealing segment, in which the reaction temperature is ramped down to a temperature at which the primers can bind to the DNA strand. The final segment is an extension segment, in which the reaction is heated to a temperature that is optimal for extension of the primer by the DNA polymerase. These three segments are cycled through multiple times allowing for the production of the complementary strand of DNA that pairs with the reverse transcribed DNA and of the reverse transcribed strand by extension from the forward and reverse primer or primers respectively. In each successive round of the amplification reaction, more of each DNA strand is produced, which then may be used as template for the next cycle, resulting in amplification of the DNA product. A skilled person can readily determine the appropriate temperature for each segment of the amplification step and the desired number of cycles to be performed.

In one embodiment, the amplification reaction can be started with a “hot start” in which the template DNA from the reverse transcription reaction and the forward and reverse primers are mixed and held at a temperature of the denaturation step for a period of time to reduce non-specific binding of the primers to the reverse transcribed DNA strand. One component necessary for the reaction, for example the DNA polymerase, may be omitted from the reaction during the hot start and then added to the reaction just prior to the first cycle of the amplification reaction.

If desired, the amplification step can be repeated, using the amplified DNA product as a template for an additional round of amplification cycles. The template may be purified from the reaction mixture, and a second reaction may be set up with the amplified DNA product, the appropriate primers, DNA polymerase, buffer, and deoxynucleotides. The second round of amplification may be carried out under the same or similar conditions as the first amplification reaction, and the second amplification product can then be detected using an appropriate detection method as set out below.

If a primer or primers of the invention were used in the reverse transcription reaction, the same reverse primer or primers may be used in the amplification reaction along with a suitable forward primer or primers. Alternatively, a different reverse primer or primers of the invention may be used in the amplification reaction, each primer comprising any one of the sequences set out in SEQ ID NO:32 to SEQ ID NO:71, SEQ ID NO:113 and SEQ ID NO:114 or SEQ ID NO:94 to SEQ ID NO:111 and SEQ ID NO:118.

The forward primers directed against conserved regions of the HA gene of avian influenza virus subtype H5 or H5N1 are set out in Table 1, and the forward primers directed against conserved regions of the NA gene of the H5N1 subtype are set out in Table 3. A skilled person will understand that the forward and reverse primers used in a particular amplification reaction need to correspond with respect to subtype and gene. Therefore, when a reverse primer is used that comprises any one of SEQ ID NO:32 to SEQ ID NO:55 and SEQ ID NO:113, a forward primer may be used that comprises any one of SEQ ID NO:1 to SEQ ID NO:18. Similarly, when a reverse primer is used that comprises any one of SEQ ID NO:56 to SEQ ID NO:71 and SEQ ID NO:114, a forward primer may be used that comprises any one of SEQ ID NO:19 to SEQ ID NO:31 and SEQ ID NO:112, and when a reverse primer is used that comprises any one of SEQ ID NO:94 to SEQ ID NO:111 and SEQ ID NO:118, a forward primer may be used that comprises any one of SEQ ID NO:72 to SEQ ID NO:93.

It will be appreciated that only one of the forward or reverse primers used in an amplification reaction need comprise the sequence of any one of SEQ ID NO:1 to SEQ ID NO:114, and that such a primer may be used in combination with a reverse or forward primer that does not comprise such a sequence. However, since the sequences of the HA and NA genes may vary considerably among viral isolates, using a forward or reverse primer that does not comprise the sequence of any one of SEQ ID NO:1 to SEQ ID NO:114 may affect the sensitivity and detection range of the amplification step.

As with the reverse primer, where a family of forward primers is available according to the present invention, one or more of such primers may be used so as to enable identification of any of a wide number of subtype H5 or H5N1 isolates or variants. For example, one or more of primers having the sequence set out in SEQ ID NO:1 to SEQ ID NO:6 may be used in a single amplification reaction.

One or more reverse primers may be chosen from primers comprising SEQ ID NO:32 to SEQ ID NO:71, SEQ ID NO:113 and SEQ ID NO:114 or SEQ ID NO:94 to SEQ ID NO:111 and SEQ ID NO:118, and one or more forward primers may be chosen from primers comprising SEQ ID NO:1 to SEQ ID NO:31 and SEQ ID NO:112 or SEQ ID NO:72 to SEQ ID NO:93 even where the primers do not fall within a family of primers. However, this will result in a series of amplification of products of varying lengths. If the multiple reverse and/or forward primers are carefully chosen, amplification products may be readily distinguishable from each other. It should be noted that in this embodiment, the sensitivity of the detection method may be reduced, yielding less of a particular amplification product from a given amount of template. As in the reverse transcription reaction, where more than one primer of the invention is used each of the primers used will have a different sequence, the sequence of each primer comprising any one of SEQ ID NO:32 to SEQ ID NO:71, SEQ ID NO:113 and SEQ ID NO:114 or SEQ ID NO:94 to SEQ ID NO:111, SEQ ID NO:112 and SEQ ID NO:118 for the reverse primers and any one of SEQ ID NO:1 to SEQ ID NO:31 or SEQ ID NO:72 to SEQ ID NO:93 for the forward primers.

The forward primer is chosen such that in combination with the reverse primer used, a detectable double-stranded DNA amplification product is produced. That is, the forward primer should be located sufficiently upstream in the HA or NA gene relative to the reverse primer to amplify a double stranded DNA molecule that is of sufficient size such that when produced in the amplification reaction, it is capable of being detected by whichever detection method is chosen. The size of DNA product that can be detected will vary with the specific detection method chosen. For example, if agarose gel electrophoresis is used to detect the amplification product, the end product may have to be larger than if real time PCR using lightcycling is used as the detection method. Depending on the concentration of gel used, agarose gel electrophoresis can be used to detect fragments as small as 25 base pairs. However, larger fragments, for example between 150 to 500 base pairs, are more readily detected using gel-based methods, whereas smaller fragments, for example, less than 100 base pairs are easily detected using real time PCR methods.

The amplified DNA product may be detected using detection methods known in the art. For example, suitable detection methods include, without limitation, incorporation of a fluorescent, chemiluminescent or radioactive signal into the amplified DNA product, or by polyacrylamide or agarose gel electrophoresis, or by hybridizing the amplified product with a probe containing an electron transfer moiety and detecting the hybridization by electronic detection methods.

In one embodiment, the amplified DNA product is detected by agarose gel electrophoresis, which will be known to a skilled person. A portion of the amplification product is mixed with appropriate gel loading buffer, including dye markers, and run through an agarose gel through the application of an electrical gradient to the gel. The agarose gel may be stained with ethidium bromide or another suitable dye that binds to or intercalates with DNA, and is detected for example, by exposing to ultraviolet radiation.

The detection method may be performed subsequent to the amplification reaction. Alternatively, the detection method may be performed simultaneously with the amplification reaction. In one embodiment, the amplified DNA product is detected using real time PCR, for example by lightcycling, for example using Roche's LightCycler™. Real time PCR techniques will be known by a skilled person and may involve the use of two probes each labelled with a specific fluorescent label, and which bind to the amplified DNA product. The probes are designed such that they bind to the DNA product in such a manner that the fluorescent label of the first probe is in close proximity to the fluorescent label of the second probe. The amplification reaction is performed in an instrument designed to emit and detect the relevant fluorescent signals, and includes an additional detection segment in which the instrument emits light at a wavelength suitable to excite the fluorescent label on the first probe, which then emits light at a wavelength suitable to excite the fluorescent label on the second probe. The light which is then emitted by the second probe's fluorescent label, and which differs in wavelength from the previous emissions, is detected by the instrument.

Alternatively, a fluorescent molecule that binds to double stranded DNA may be used where a single stranded template is used in the amplification reaction. This method allows for detection and fairly precise relative quantification, when compared with a known standard template, of the amplified DNA product throughout the amplification reaction. The quantification of amplified product may enable the determination of viral load in the original biological sample. As well, this method allows for the detection of smaller amounts of amplification products, and amplification products having smaller sizes than methods using conventional PCR techniques.

The simultaneous amplification and detection may also be performed using a detection probe that is labelled at the 5′end with a fluorophore and at the 3′ end with a quenching molecule that quenches emissions of the fluorophore when in proximity to the fluorophore, as in the Taqman™ method designed by ABI Systems. The detection probe will bind to the forward or reverse strand of the amplification template. A polymerase having 5′ exonuclease activity, for example, Taq polymerase or others (for example, synthetic version is available from Roche), is used in the amplification reaction. As the template strand having the bound detection probe is amplified, the detection probe will be digested by the 5′ exonuclease, removing the fluorophore from the proximity of the quencher and allowing the fluorophore to emit. The emissions can be quantified in standard equipment, for example, the LightCycler™ described above.

Alternatively, to detect H5N1 variants, a first amplification may be performed using primers directed against the HA gene, for example, using reverse primer or primers comprising any one of SEQ ID NO:32 to SEQ ID NO:71, SEQ ID NO:113 and SEQ ID NO:114 and forward primer or primers comprising any one of SEQ ID NO:1 to SEQ ID NO:31 and SEQ ID NO:112, and a second amplification step may be performed using reverse primer or primers comprising any one of SEQ ID NO:94 to SEQ ID NO:111 and SEQ ID NO:118 and forwards primer or primers comprising any one of SEQ ID NO:72 to SEQ ID NO:93 directed against the NA gene. To detect the presence of H5N1, the two amplifications may be performed simultaneously, in the same or different reaction, the first amplification using primers directed against the H5 or H5N1 subtype of the HA gene, and the second amplification using primers directed against the NA gene. As the NA gene is known to be expressed in lower quantities, it may be more difficult to detect in instances of low viral load.

The large number of primers provided by the present invention are designed to increase the possibility of detecting different variants of subtypes H5 and H5N1, and a single sample may be tested with different combinations of forward and reverse primer or primers, so as to increase the probability of detecting any particular variant.

Although the above embodiments have been described in the context of a PCR amplification method, a skilled person will understand that the sequences of the invention may be used to design primers for use in other amplification methods to detect avian influenza virus subtype H5 or H5N1 in a biological sample. For example, the sequences disclosed in SEQ ID NO:1 to SEQ ID NO:114 may be used to design primers for amplification and detection by NASBA methods, as described for example in Lau et al. (Biochem. Biophys. Res. Comm. 2003 313:336-342), and which are generally known to a skilled person.

Briefly, in the NASBA technique the primers are designed to bind to a portion of the gene of interest, here HA or NA, and to include a promoter for an RNA polymerase, for example T7 RNA polymerase. The viral gene is reverse transcribed and a second complementary DNA strand is synthesized to produce a double stranded DNA molecule that includes an intact RNA polymerase promoter. The relevant RNA polymerase is used to generate copies of an RNA molecule corresponding to an amplified portion of the gene of interest. The amplified RNA is then bound to a detection molecule, typically a nucleic acid that is complementary to a portion of the amplified RNA and that is labelled, for example, with a radiolabel, a chemiluminescent label, a fluorescent label or an electrochemiluminescent label. The amplified RNA bound to the detection molecule is then typically captured by a capture molecule, for example an immobilized nucleic acid that is complementary to a portion of the amplified RNA product that is a different portion than that to which the detection molecule binds. The captured RNA amplification product with bound detection molecule is then detected by the relevant detection method as determined by the label on the detection molecule and the method of capture.

Thus, the present invention contemplates the use of a primer comprising any one of SEQ ID NO:1 to SEQ ID NO:114 for use in NASBA methods to detect the presence of avian influenza virus subtype H5 or H5N1 in a biological sample.

The primers of the invention are also useful for sequencing a DNA molecule corresponding to the HA or NA gene, or a reverse transcribed DNA molecule complementary to the HA or NA gene of the avian influenza virus subtype H5 or H5N1. A reverse primer comprising any one of SEQ ID NO: 32 to SEQ ID NO:71, SEQ ID NO:113 and SEQ ID NO:114, or any one of SEQ ID NO:94 to SEQ ID NO:111 and SEQ ID NO:118 may be used to initiate a sequencing reaction using as template nucleic acid molecule corresponding to a portion of the HA or NA gene, respectively. A forward primer comprising any one of SEQ ID NO:1 to SEQ ID NO:31 and SEQ ID NO:112 or any one of SEQ ID NO:72 to SEQ ID NO:93 may be used to initiate a sequencing reaction using as template a nucleic acid molecule complementary to a portion of the HA or NA gene, respectively. Sequencing reactions may be performed using standard methods known in the art, and may be performed using automated sequencing equipment.

The primers of the invention are also useful as probes or capture molecules to detect RNA from an H5 or H5N1 influenza virus isolate. For example, one or more primers comprising any one of SEQ ID NO:1 to SEQ ID NO:114 may be immobilized on a solid support and used to isolate nucleic acid molecules having a sequence that is complementary to some or all of the primer sequence.

Thus, there is presently provided a method for detecting influenza A virus subtype H5 or H5N1 in a sample comprising contacting one or more immobilized primers comprising any one of the sequences of SEQ ID NO:1 to SEQ ID NO:114 with the sample.

The primer may be immobilized on a solid support using standard methods for immobilizing nucleic acids, including chemical cross-linking, photocross-linking, or specific immobilization via a functional group on the primer, including a functional group that is added to or incorporated into the primer, for example biotin.

The solid support may be any support which may be used in a detection assay, including chromatography beads, a tissue culture plate or dish, or a glass surface such as a slide.

The contacting is performed under conditions that allow for hybridization between the primer and the sample so that any nucleic acids contained in the sample that contain a sequence complementary to the primer or to a portion of the primer can hybridize. A skilled person will be able to determine suitable hybridization conditions based on the sequence of the primer or the region of the primer that is to be hybridized with the sample, and will be able to vary conditions so as to increase or decrease the stringency of hybridization. For example, varying of temperature, salt or buffer concentrations and detergent concentrations will alter the stringency of conditions for hybridization between a given sequence and its complement.

In such an application, the primer or the nucleic acid sample, or both, may be modified with a label such as a fluorescent label, a chemiluminescent label, a coloured dye label, a protein, peptide or ligand.

Any hybridized nucleic acids from the sample that have been captured by immobilized primer are then detected. The method of detection will depend on the nature of any label on the sample and/or the immobilized primer. As well, standard methods for detecting and visualizing nucleic acid molecules may be used, including chromatography methods, and gel electrophoresis and staining methods.

One example of an immobilization and capture application is incorporation of the primer or primers in a DNA or nucleotide microarray, as is known in the art.

Thus, there is also provided a method of detecting influenza A virus subtype H5 or H5N1 in a sample comprising contacting a microarray containing one or more primers comprising any one of the sequences of SEQ ID NO:1 to SEQ ID NO:114 in at least one spot in the microarray with the sample, and detecting hybridization of the sample to the primer. Nucleic acid microarray technology is known in the art, including manufacture of a microarray and detection of hybridization of a sample with the capture molecules in one or more spots in the microarray.

The sample used may be a biological sample suspected of containing influenza A virus subtype H5 or H5N1, or containing nucleic acid generated or amplified from a biological sample suspected of containing influenza A virus subtype H5 or H5N1. Nucleic acid may be amplified using known amplification methods as described above, including RT-PCR amplification as described herein, the NASBA method described herein, as well as primer extension methods. The sample may be hybridized with the primer or primers incorporated into the microarray using known hybridization methods.

In order to detect hybridization, typically either the primer, which acts as a probe, or the sample is labelled. For example, the sample may be labelled by incorporating a label during an amplification step. The label may be any detectable label, including a radioactive label, a chemiluminescent label or a fluorescent label. For example, as is known in the art, Cy3- or Cy5-labelled nucleotides can be readily incorporated into an amplified nucleic acid molecule to generate a labelled sample. Using this method, hybridization is typically detected by an increase in signal at a particular spot in the microarray.

Alternatively, the presently described primers which are included in the microarray may be labelled. For example, U.S. Pat. No. 6,811,973 (issued to Reich), which is herein fully incorporated by reference, describes inclusion of a fluorescent marker into a nucleic acid probe molecule that is used as a capture molecule in a microarray, whereby hybridization of the capture nucleic acid molecule with its target nucleic acid molecule results in quenching of the fluorescent signal emitted by the fluorescent label incorporated into the capture molecule. Thus, hybridization is measured by a decrease in signal from a particular spot in the microarray.

The present invention also contemplates microarrays incorporating one or more primers comprising any one of the sequences of SEQ ID NO:1 to SEQ ID NO:114 at one or more spots in the microarray. Methods for manufacturing microarrays, including nucleic acid microarrays are known. For example, U.S. Pat. No. 6,753,144 (Hirota) and U.S. Pat. No. 6,511,849 (Wang), both of which are fully incorporated by reference herein, describe methods for making microarrays. The microarray is typically formed on a solid support by immobilizing one or more primers at a given address or spot in the microarray.

As with the amplification, one or more members of a family of primers may be included in a single spot, allowing for detection of a number of different variants or isolates with a single spot in the microarray.

Although the methods described above relate to in vitro methods of detecting influenza A virus H5 or H5N1 isolates, the primers described herein may also be used in vivo methods to detect or image an influenza A virus H5 or H5N1 subtype infection.

Also presently contemplated are kits or commercial packages comprising a primer as described above and instructions for detecting influenza A H5 or H5N1 subtype in a sample. The detecting may be by any of the methods described herein.

All documents referred to herein are fully incorporated by reference.

### EXAMPLES

Experiments were performed on RNA extracted from eggs and from human clinical samples including allantoic fluid, cloacal and trachael swabs, homogenized tissue, pooled organs, blood, sputum, stools, urine and nasopharyngeal aspirates.

### Example 1

### Detection of Avian Influenza Virus H5 and H5N1 Using Gel-Based Detection Platform

The following is a general protocol for detection of avian influenza virus subtype H5 or H5N1.

Generally, RNA is extracted from samples according to the manufacturer's instructions, using either TRIzol™ or RNA extraction kits (Qiagen).

The first-strand cDNA synthesis is performed on extracted RNA using the relevant reverse primer(s) (2 μl of 10 μM stock) in a 20 μl reaction volume. A first round PCR reaction is set up using 2.5 μl of the cDNA reaction, containing cDNA product as template with relevant forward and reverse primer(s) (1.25 μl total volume for each of forward and reverse) in a 25 μl reaction volume. The PCR conditions are set up as follows: incubation at 94° C. for 2 min; 35 cycles of 94° C. for 10 sec, 50° C. for 30 sec, 72° C. for 1 min; followed by an incubation at 72° C. for 7 min. A second round of PCR is performed using the product of the first round PCR (2.5 μl) as template. All other conditions and reagents are the same as for the first round PCR.

The products of the second round PCR are analysed on a 1.5 to 2% agarose gel by staining with ethidium bromide.

However, it has been established after rounds of validation that one-round of PCR will be sufficient for detection

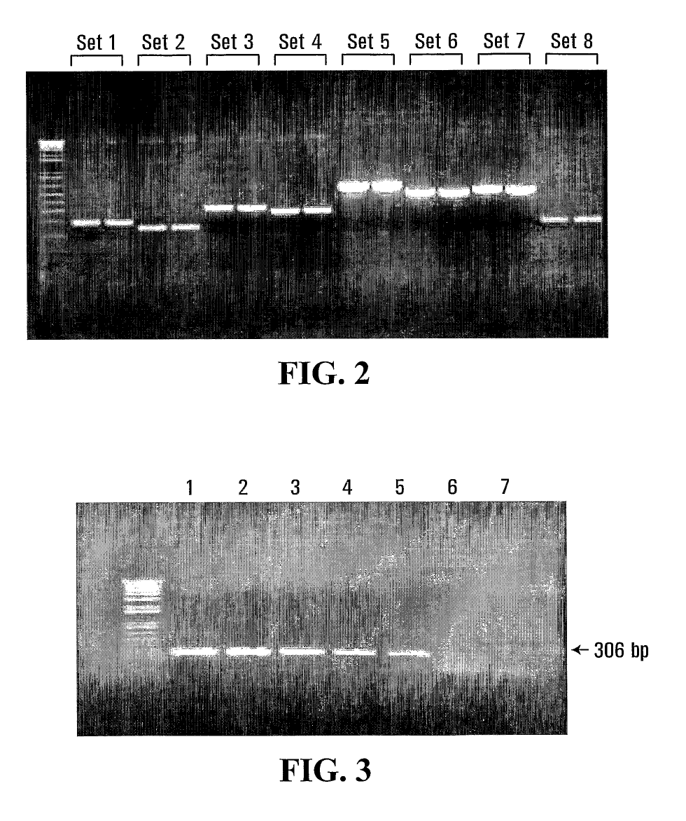

The above RT-PCR protocol was performed using RNA extracted from an H5N1 viral isolate derived from Thailand (2004). The following 8 primer sets directed against the HA gene were used: (1) gisAFH5N1H1F and gisAFH5N1H4R; (2) gisAFH5N1H2aF and gisAFH5N1H4R; (3) gisAFH5N1H1F and gisAFH5N1H5bR; (4) gisAFH5N1H2aF and gisAFH5N1H5bR; (5) gisAFH5N1H3F and gisAFH5N1H8aR; (6) gisAFH5N1H6bF and gisAFH5N1H8aR; (7) gisAFH5N1H6bF and gisAFH5N1H10R; (8) gisAFH5N1H7aF and gisAFH5N1H11cR. The expected fragment sizes were 189 bp, 148 bp, 306, bp, 265 bp, 574 bp, 456 bp, 489 bp and 163 bp, respectively. The amplified products were run on a 1.5% agarose gel and visualized by ethidium bromide staining, as shown in FIG. 2.

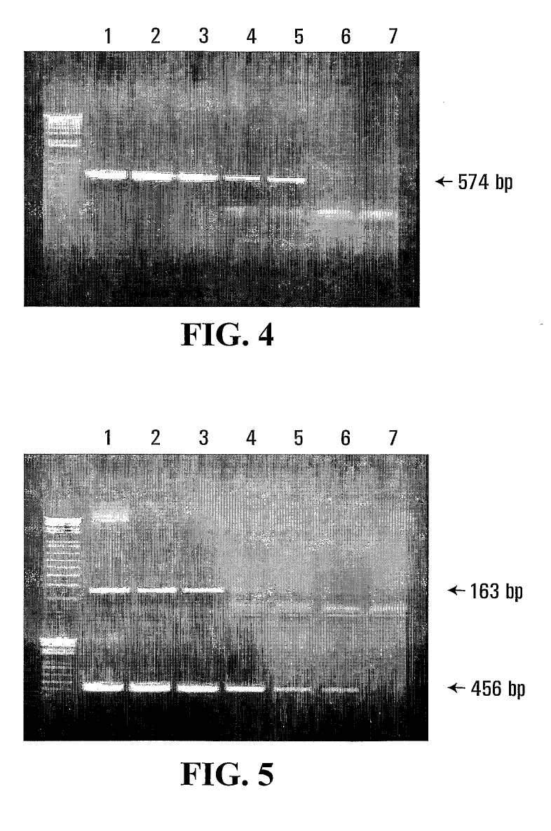

To determine the sensitivity of this assay, reactions using 10 fold serial dilutions of template and primer sets 3 (FIG. 3), 5 (FIG. 4), 6 and 8 (FIG. 5) were performed. The results (FIGS. 3 to 5; lanes 1 to 7 are reactions performed using 50 ng, 0.5 ng, 50 pg, 0.5 pg, 0.05 pg, 5 fg and 0.5 fg of template, respectively) indicate that the PCR reactions using the primers of the invention are sensitive, although the sensitivity varies depending on the primers used. Primer sets 3 and 6 were more sensitive, and primer set 6 yielded a significant amount of amplified product, even at a template concentration as low as 5 fg (see lane 6, FIG. 5).

### Example 2

### Detection of Avian Influenza Virus H5 and HSN1 Using Real-Time RT-PCR Detection Platform

The following reactions are performed in a LightCycler™ instrument.

The reaction master mixture is prepared on ice by mixing the following reagents in order, to a volume of 20 μl: water (volume adjusted as necessary), 50 mM manganese acetate (1.3 μl), ProbeNPrimer mix containing forward primer and reverse primer to a final concentration of 0.2 to 1 μM and fluorescently labelled probes (2.6 μl), LightCycler RNA Master Hybridization Probes (7.5 μl), which contains buffer, nucleotides and enzyme.

The reactions are transferred to glass capillary tubes suitable for use in the LightCycler™. 5 μl of extracted RNA template is added to each reaction and briefly centrifuged. The RT-PCR reactions are run using the following programs (Tables 5-8):

TABLE 5
Program 1-Reverse Transcription

| Cycle Program Data | Value |
|---|---|
| Cycles | 1 |
| Analysis Mode | None |
| Temperature Targets | Segment 1 |
| Target T° C. | 61 |
| Incubation time | 20 min |
| T° C. transition rate (° C./s) | 20.0 |
| Secondary Target T° C. | 0 |
| Step Size (° C.) | 0.0 |
| Step Delay (cycles) | 0 |
| Acquisition Mode | None |

TABLE 6
Program 2-Denaturation

| Cycle Program Data | Value |
|---|---|
| Cycles | 1 |
| Analysis Mode | None |
| Temperature Targets | Segment 1 |
| Target T° C. | 95 |
| Incubation time | 30 sec |
| T° C. transition rate (° C./s) | 20.0 |
| Secondary Target T° C. | 0 |
| Step Size (° C.) | 0.0 |
| Step Delay (cycles) | 0 |
| Acquisition Mode | None |

TABLE 7
Program 3-Amplification

| Cycle Program Data | Value |
|---|---|
| Cycles | 1 |
| Analysis Mode | Quantification |

| Temperature Targets | Segment 1 | Segment 2 | Segment 3 |
|---|---|---|---|
| Target T° C. | 95 | 50 to 55 | 72 |
| Incubation time | 5 sec | 15 sec | 13 sec |
| T° C. transition rate (° C./s) | 20.0 | 20.0 | 2.0 |
| Secondary Target T° C. | 0 | 0 | 0 |
| Step Size (° C.) | 0.0 | 0.0 | 0.0 |
| Step Delay (cycles) | 0 | 0 | 0 |
| Acquisition Mode | None | Single | None |

TABLE 8
Program 4-Cooling

| Cycle Program Data | Value |
|---|---|
| Cycles | 1 |
| Analysis Mode | None |
| Temperature Targets | Segment 1 |
| Target T° C. | 40 |
| Incubation time | 30 sec |
| T° C. transition rate (° C./s) | 20.0 |
| Secondary Target T° C. | 0 |
| Step Size (° C.) | 0.0 |
| Step Delay (cycles) | 0 |
| Acquisition Mode | None |

### Example 3

### Detection of Avian Influenza Virus H5N1 Using Real-Time RT-PCR with Various Primer Sets

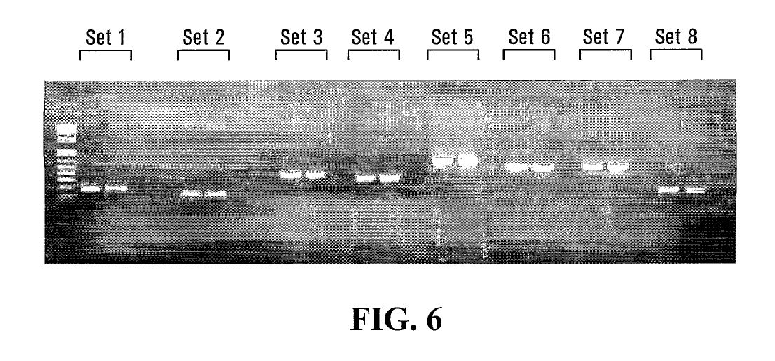

Real time PCR reactions were performed using the 8 primer sets described in Example 2 above. The reactions were performed using SYBR green fluorescent detection kit, in accordance with standard protocols and commercially available reagent kits (Roche). FIG. 6 displays the amplification products obtained for the reactions performed with each of the 8 primer sets as visualized on a 1.5% agarose gel stained with ethidium bromide.

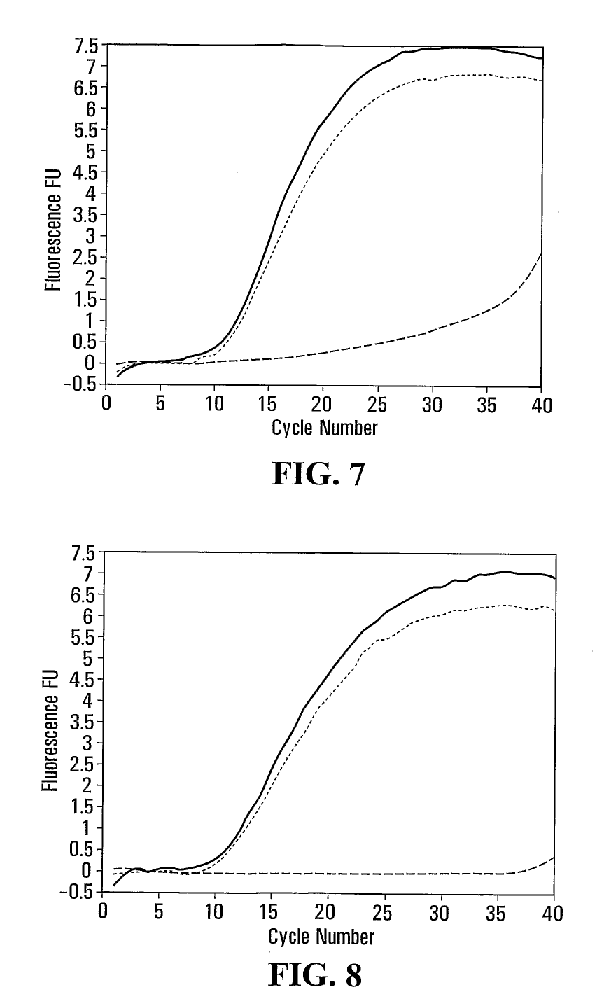

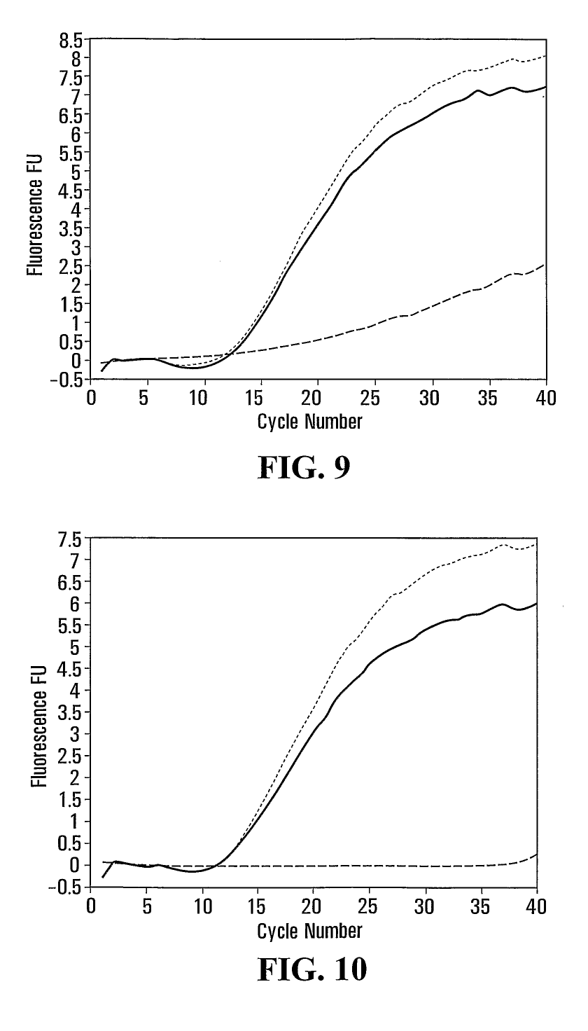

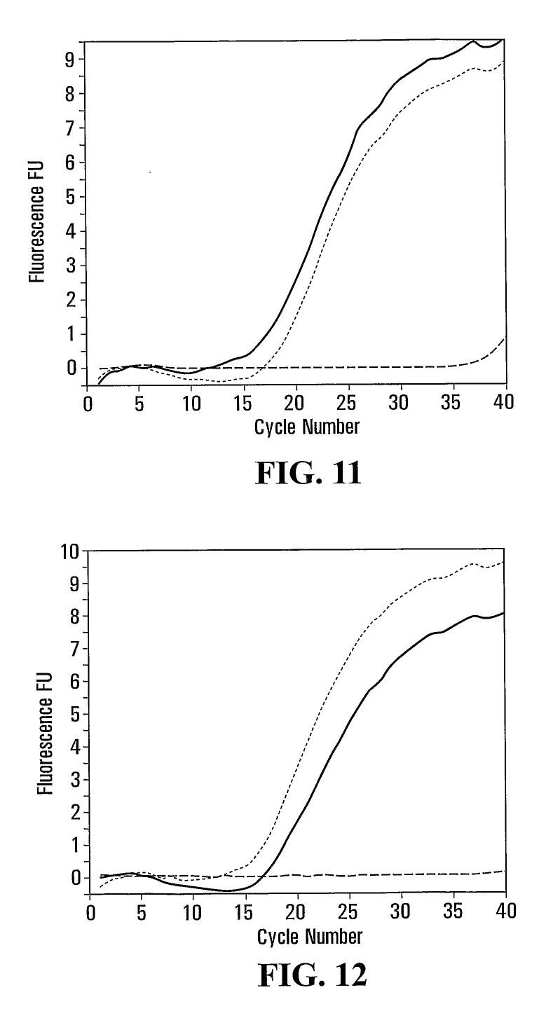

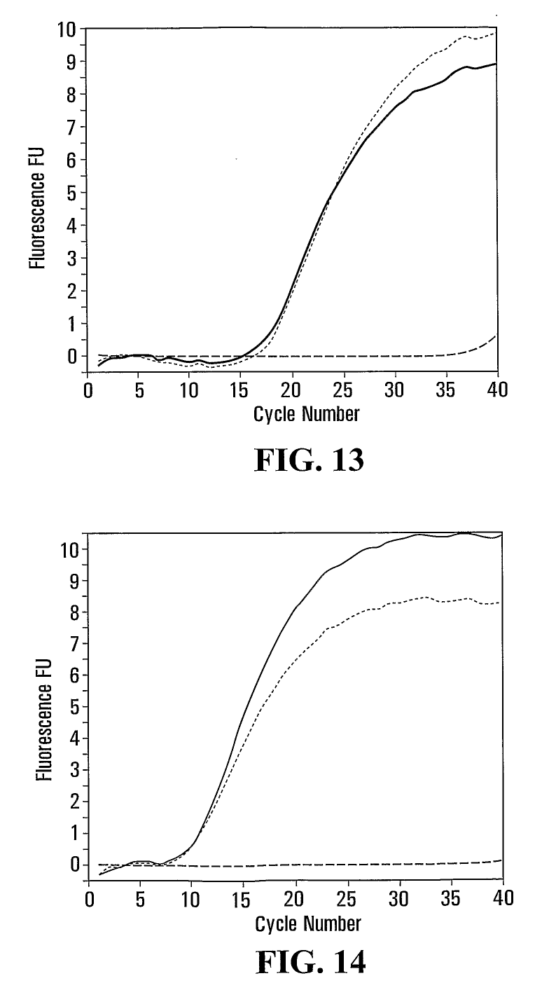

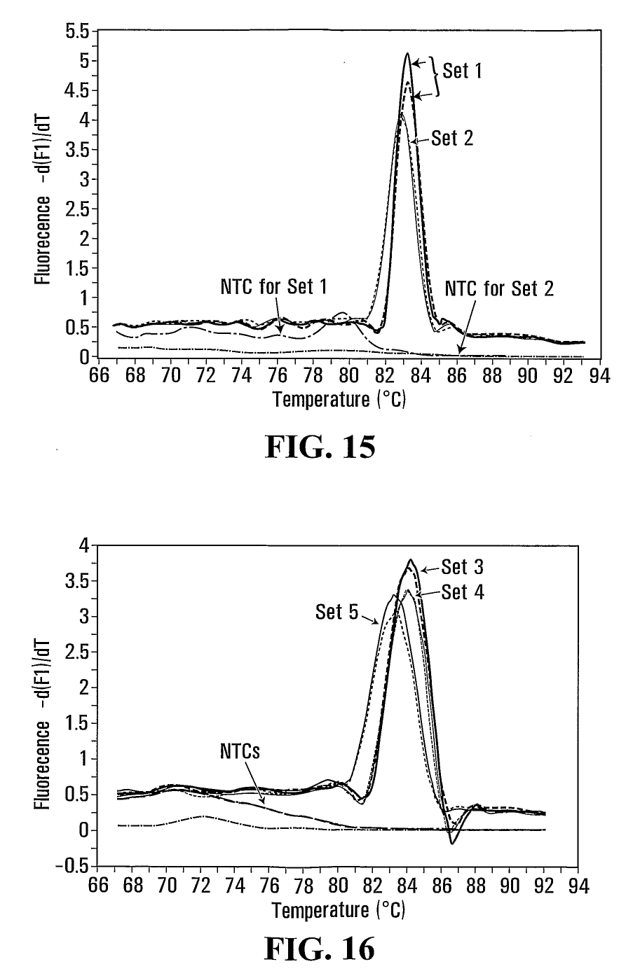

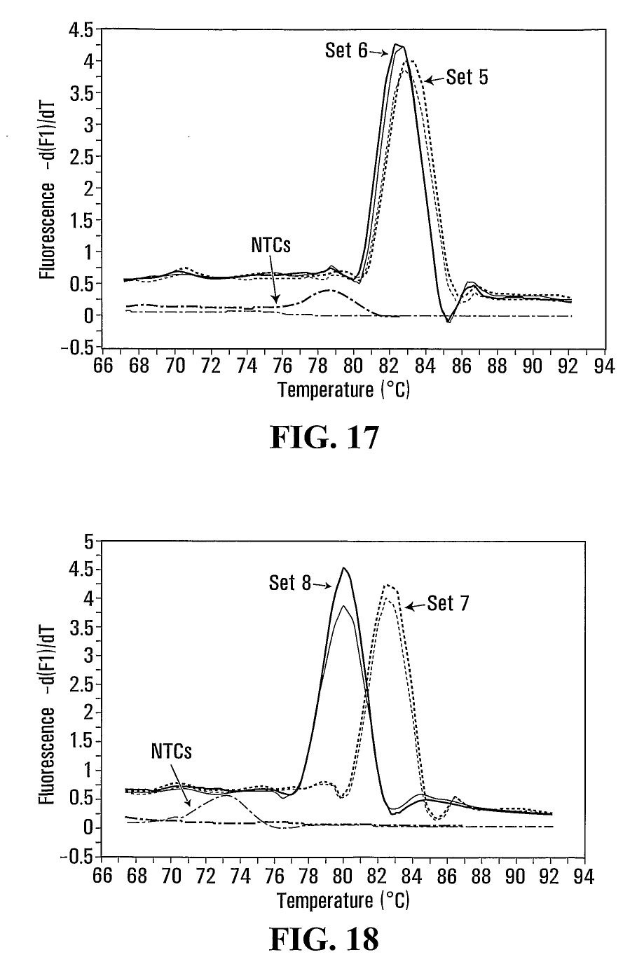

To confirm the sensitivity of the primers using the real time PCR protocol, amplification curves were generated to monitor the production of amplification product. Results are shown in FIGS. 7 to 14 for each of primer sets 1 to 8, respectively. Melting curves of the amplified product were performed at the end of the amplification reaction. Generally, specific amplification products will have a higher melting temperature than non-specific products, and the melting curve profile can be used to confirm the specificity of the reaction. The melting curves shown in FIGS. 15 to 18 are indicative of discrete expected amplification products. Thus, the primers of sets 1 to 8, when used in real time PCR reactions are highly sensitive and specific for H5N1 isolates.

### Example 4

### Detection of H5N1 Influenza Virus from Field Samples Using One-Step RT-PCR Reaction

The performance of the primers was assessed using primer set 6 (described above in Example 1) in gel-based assays using in vitro-transcribed RNA generated by the T7 RiboMax Express in vitro transcription system (Promega, USA). The concentration of purified transcribed RNA was measured by RiboGreen RNA quantitation reagent (Invitrogen, USA) and serial dilutions of in vitro-transcribed RNA were prepared in duplicate.

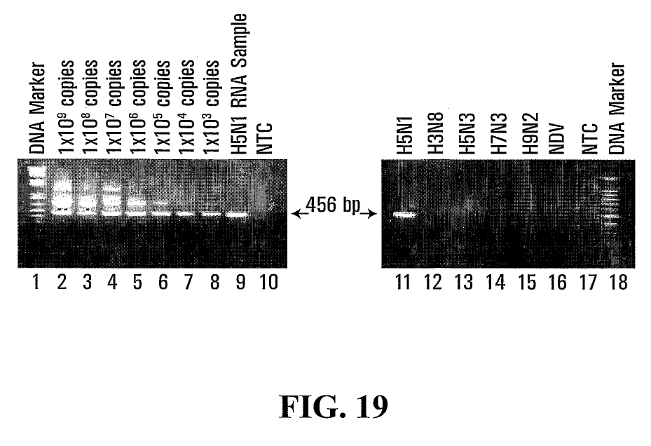

2 μl of RNA was used in 25 μl reaction mixtures using the One-Step reverse transcription (RT)-PCR system (Qiagen, Germany) with the H5N1 specific primers (set 6) using the following PCR cycle: 94° C. for 10 sec; followed by 35 cycles of: 94° C. for 10 sec, 50° C. for 30 sec, and 72° C. for 1 min; and lastly followed by 72° C. for 7 min. The size of this PCR product was 456 bp and was resolved in 1% agarose gels. PCR products were sequenced directly to confirm the identity of the products. The sensitivity of the assay was found to be less than 1000 copies and was able to specifically detect H5N1 RNA (FIG. 19A).

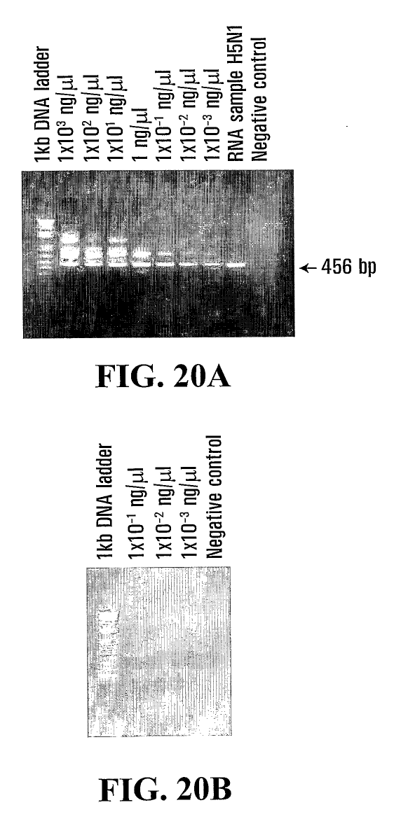

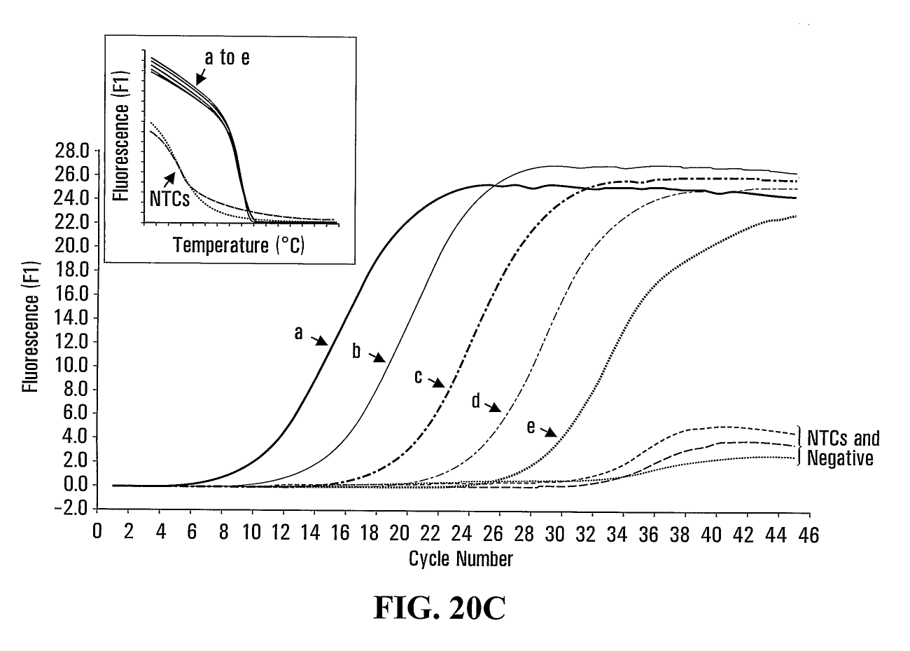

The two-step RT-PCR method (Roche, Germany) was also used as described above and compared with the one-step RT-PCR method. Results showed both systems work well (compare FIG. 20A (two-step) and 20B (one-step)). The primers were then tested using the LightCycler Real-time system (Roche, Germany) and comparable results were achieved (FIG. 20C) indicating equal sensitivities in terms of detection between the two assays.

For FIG. 20C, amplification of RNA standards (indicated as a to e) are shown. The x axis denotes the cycle number of the quantitative PCR assay, and the y axis denotes fluorescence intensity (F2) over the background level. The non-template control (NTC) is shown. RNA standards were as follows: (a) 1×10⁹ copies per reaction, (b) 1×10⁸ copies per reaction, (c) 1×10⁷ copies per reaction, (d) 1×10⁶ copies per reaction, and (e) 1×10⁵ copies per reaction. The insert graph represents the melting-curve analysis of the PCR products. Signals from RNA standards (a to e), and non-template control are shown. The x axis denotes the temperature (° C.), and the y axis denotes the fluorescence intensity over the background level.

To establish the specificity of the assays for H5N1 subtype detection, we then investigated the primers on field samples using several reference strains of different subtypes of influenza A viruses (H3N8, H5N3, H7N3 and H9N2) and Newcastle disease virus (NDV) as controls. Results showed that detection was specific to H5N1 only (FIG. 19B). Samples were extracted using TRIzol reagent (Invitrogen, USA) and QIAamp Viral RNA Mini Kit (Qiagen, Germany) according to manufacturers' instructions.

For FIG. 19, Panel A depicts the results of amplification of serially diluted in vitro-transcribed single-stranded RNA (lanes 2 to 8) measured by RiboGreen RNA quantitation reagent and H5N1 RNA extracted from allantoic fluid of infected egg. The non-template control (sterile water) is indicated as “NTC”. The viral load is indicated by the number of copies per reaction: (lane 2) 1×10⁹ copies per reaction, (lane 3) 1×10⁸ copies per reaction, (lane 4) 1×10⁷ copies per reaction, (lane 5) 1×10⁶ copies per reaction, (lane 6) 1×10⁵ copies per reaction, (lane 7) 1×10⁴ copies per reaction, and (lane 8) 1×10³ copies per reaction. The H5N1 RNA is estimated to be approximately 1×10⁶ copies per reaction.

Panel B depicts the specific detection of H5N1 avian influenza virus using reference strains of different subtypes avian influenza A viruses (lanes 12 to 15), as well as Newcastle disease virus (NDV, lane 16). The H5N1, H3N8, H9N2 and NDV isolates were isolated from field samples by the Veterinary Research Institute, Malaysia, the H5N3 and H7N5 isolates were provided by the Department of Veterinary Pathology of Tottori University, Japan. Negative signals from non-H5N1 isolates and the non-template control (water) are shown.

A total of 145 field samples comprising of known and suspect cases from chickens, ducks and muscovy ducks isolated from Vietnam and Malaysia during the 2004 to 2005 outbreak were tested for H5N1 RNA (Table 9). Samples ranged from pooled organ tissues, allantoic fluid, cloacal and tracheal swabs, yielded positive results with 100% positive detection for allantoic fluid and pooled organs (Table 9) verifying the sensitivity of this RT-PCR assay. Viral culture isolation methods were used as confirmatory tests for all positive samples (Table 9). Homogenized tissues had the lowest detection rate of 40%, while cloacal and tracheal swabs detected 66.7% of the positive H5N1 samples. This variation in detection could be due to the different efficiencies in viral RNA recovery from the different samples, with allantoic fluid fractions having the highest efficiency for H5N1 RNA recovery and detection among the samples (Table 9).

TABLE 9
Detection of avian influenza A H5N1 subtype from field samples

| | | | H5N1 viral | Virus | H5N1 RT- |
|---|---|---|---|---|---|
| Sample Type | Source | Number | culture | isolate | PCR |
| Allantoic | Vietnam | 58 | 50/58 | H5N1 | 50/58 |
| Fluid | | | | | |
| Cloacal and | Vietnam | 43 | 15/43 | H5N1 | 10/43 |
| Trachael | | | | | |
| Swab | | | | | |
| Homogenized | Vietnam | 12 | 10/12 | H5N1 | 4/12 |
| Tissue | | | | | |
| Pooled | Malaysia | 32 | 32/32 | H5N1 | 32/32 |
| Organs | | | | | |

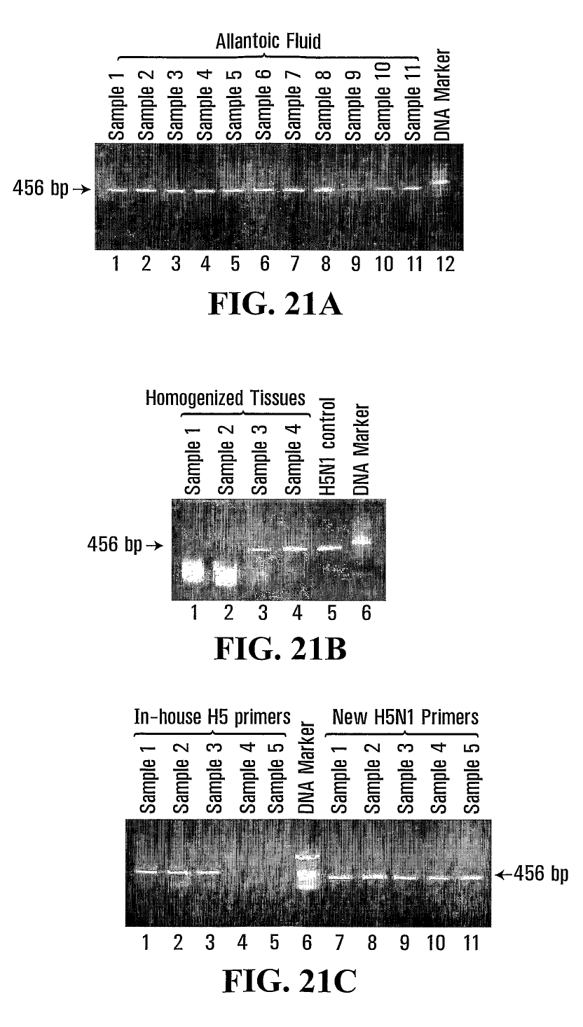

In an effort to validate the primers further, five positive samples from the Vietnam set were then tested in parallel with an existing in-house H5 primer set and results showed that the H5N1 primers described here (primer set 6) detected five out of five positive H5N1 samples while the other primer set only managed to detect three out of five (FIG. 21C), indicating that the H5N1 primers described herein are more sensitive and could detect weak positive samples with lower viral load.

In FIG. 21, the following data is depicted. Panel A shows the detection of H5N1 avian influenza virus from allantoic fluid of chickens, ducks and muscovy ducks (lanes 1 to 11). Panel B shows the detection of H5N1 avian influenza virus from homogenized tissues (lanes 1 to 4). Only two out of three samples were detected (lanes 3 and 4), which could be due to inefficient RNA extraction. The H5N1 positive control is indicated (lane 5). Panel C shows a comparison of an in-house H5 primer set with new H5N1 primers. The in-house H5 primer set detected three out of five positive samples that have been confirmed by viral culture isolation (lanes 1 to 5), while the H5N1 primer set detected all five samples (lanes to 11). The same batch of extracted RNA was used with both primer sets.

### Example 5

### Detection of H5N1 and H5 Influenza A Virus using Real Time PCR and One-Step RT-PCR Reactions

RT-PCR experiments were performed as described above using both the real-time PCR protocol and the one- or two-step PCR protocol. Experiments used in vitro-transcribed RNA.

Experiments detecting H5N1 used the following primer sets directed against the NA gene of H5N1 subtype: (9) gisAFH5N1N1aF and gisAFH5N1N3aR; (10) gisAFH5N1N1aF and gisAFH5N1N6gR; (11) gisAFH5N1N1aF and gisAFH5N1N7bR; (12) gisAFH5N1N2aF and gisAFH5N1N3aR; (13) gisAFH5N1N2aF and gisAFH5N1N6gR; (14) gisAFH5N1N2aF and gisAFH5N1N7bR; (15) gisAFH5N1N4aF and gisAFH5N1N6gR; (16) gisAFH5N1N4aF and gisAFH5N1N7bR; (17) gisAFH5N1N5gF and gisAFH5N1N7bR; (18) gisAFH5N1N8bF and gisAFH5N1N9aR; (19) gisAFH5N1N8bF and gisAFH5N1N11bR; and (20) gisAFH5N1N10aF and gisAFH5N1N11bR. The expected fragment sizes were 120 bp, 315 bp, 452 bp, 111 bp, 306 bp, 443 bp, 217 bp, 354 bp, 158 bp, 119 bp, 271 bp and 172 bp, respectively. The amplified products were run on a 1.0% or 1.5% agarose gel and visualized by ethidium bromide staining. For all reactions, QS 1=1000 ng/μl, QS 2=100 ng/μl, QS 3=10 ng/μl, QS 4=1 ng/μl, QS 5=0.1 ng/μl, QS 6=0.001 ng/μl and NTC non-template control, respectively.

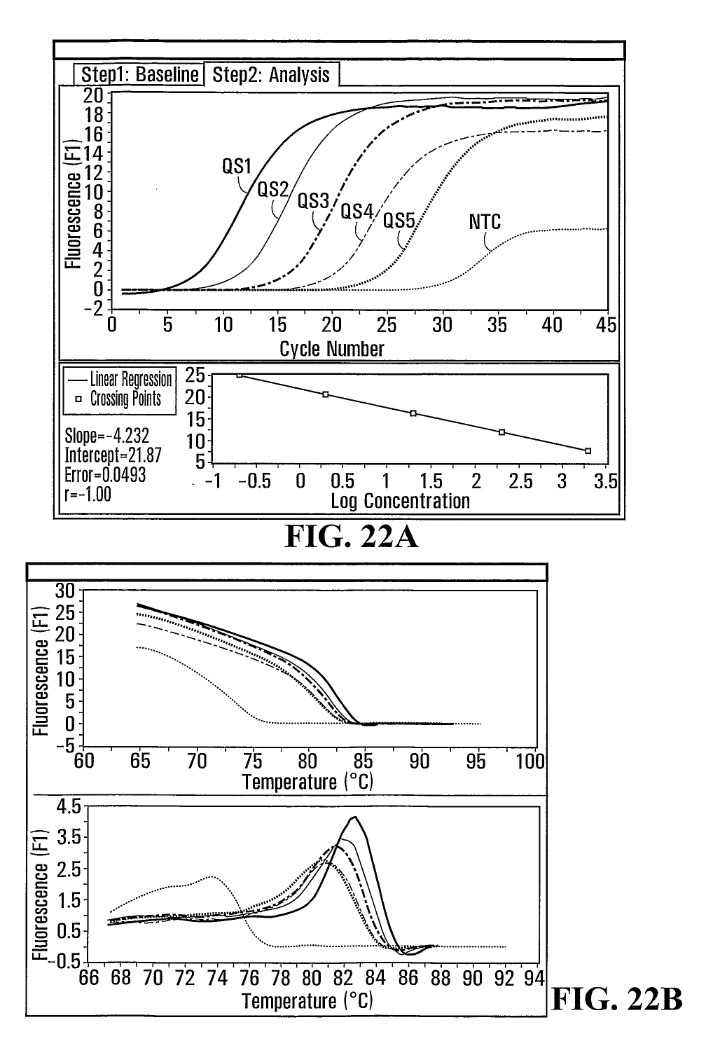

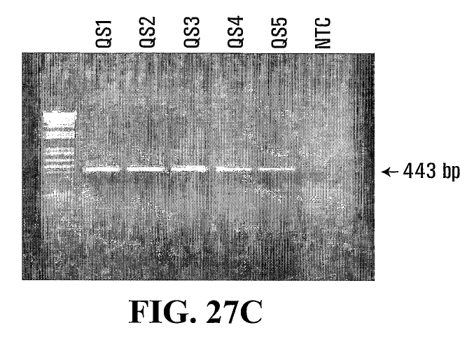

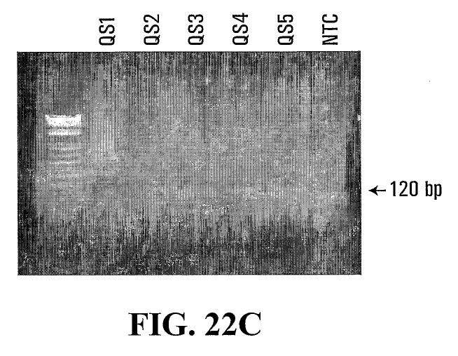

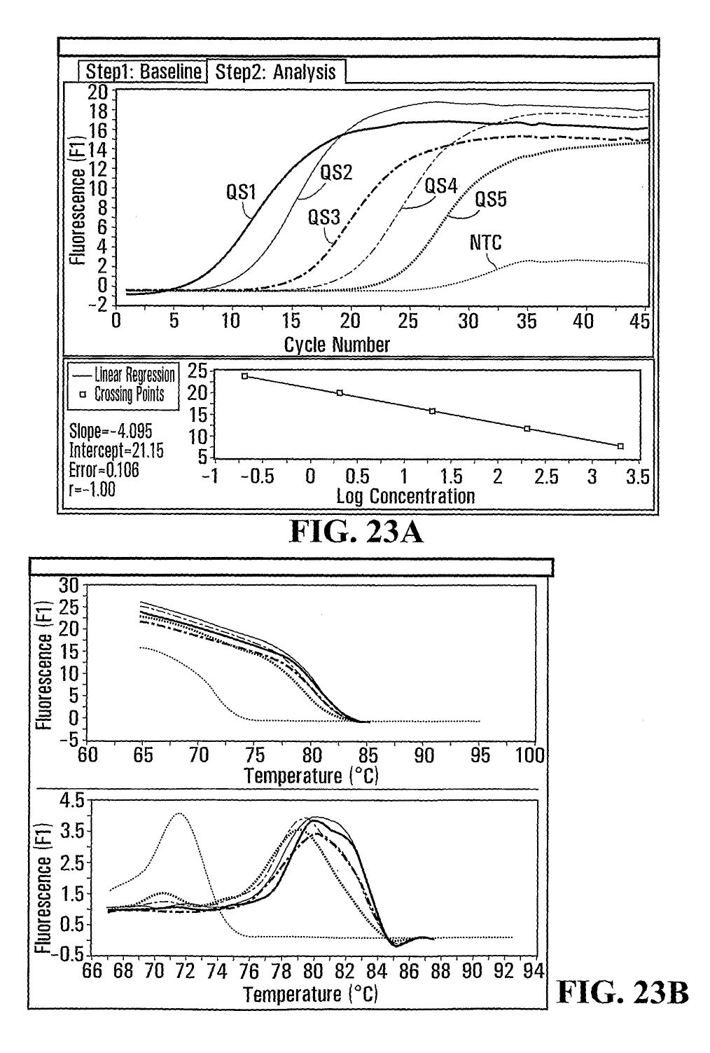

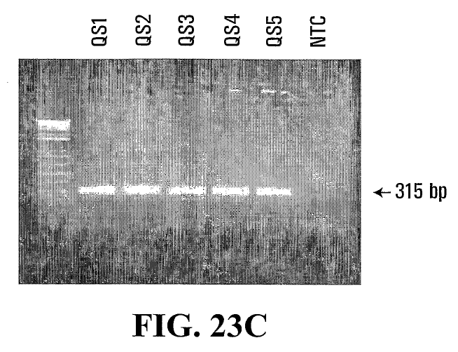

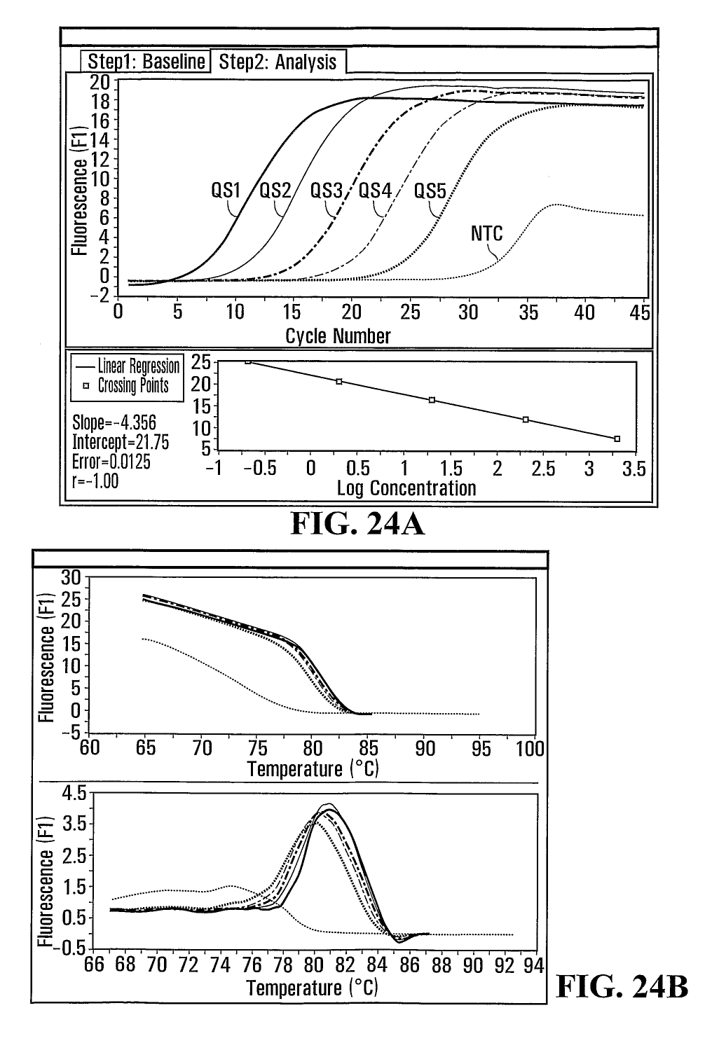

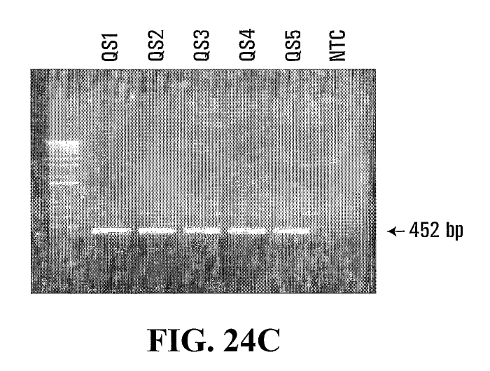

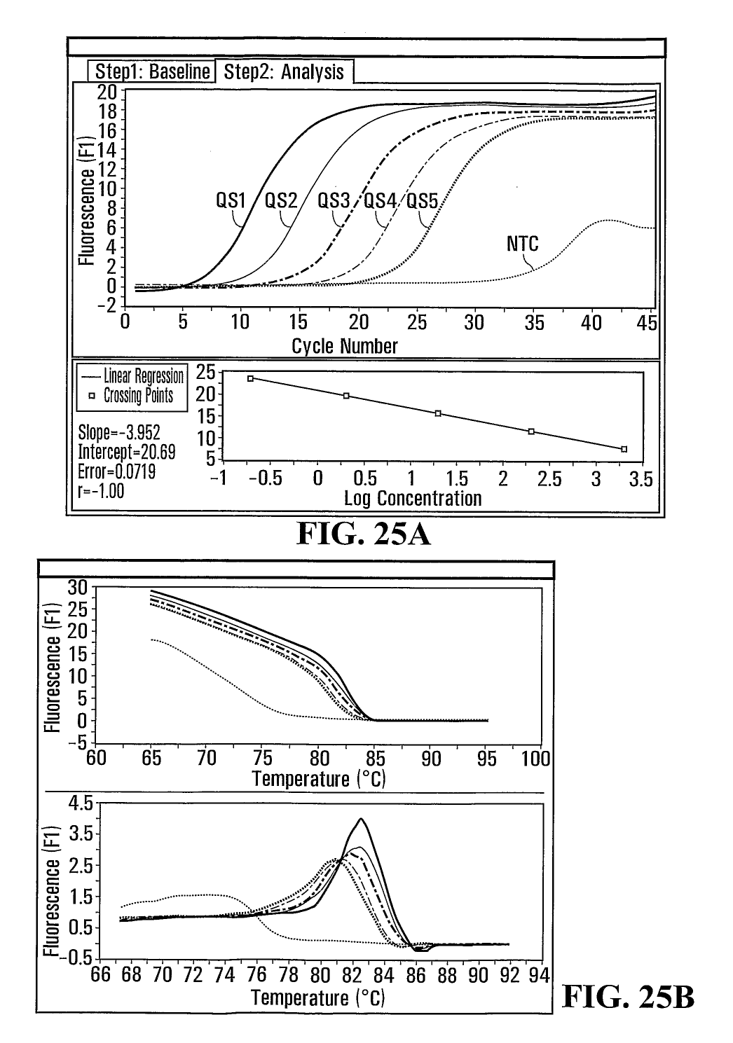

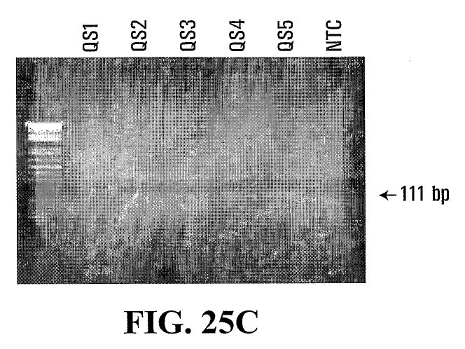

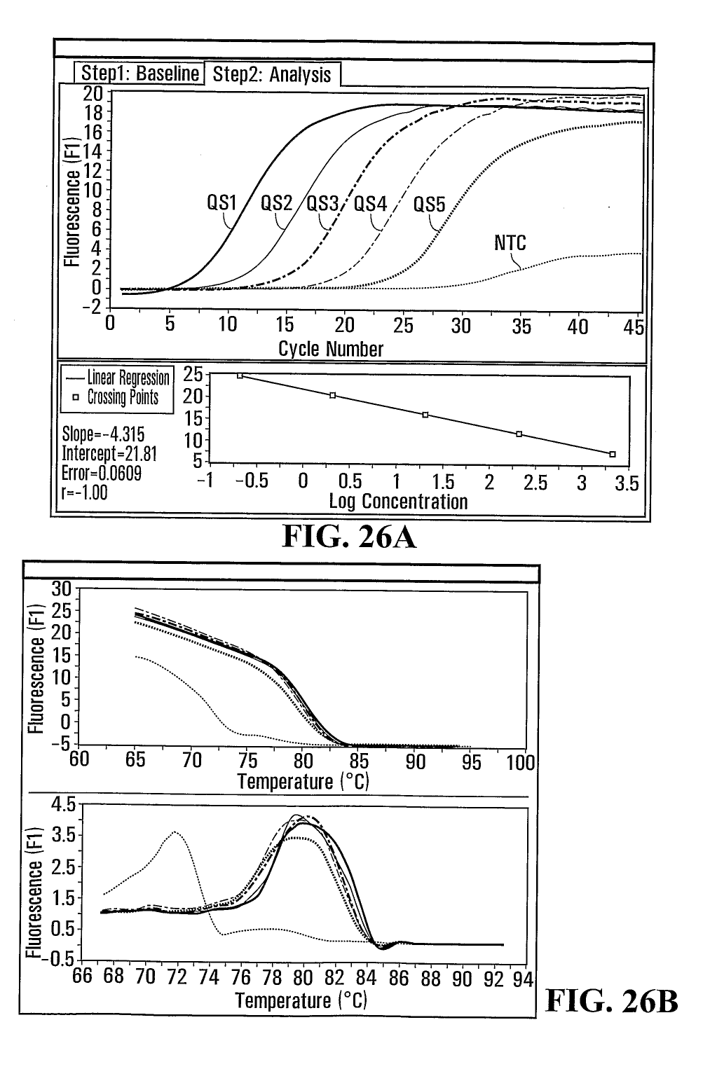

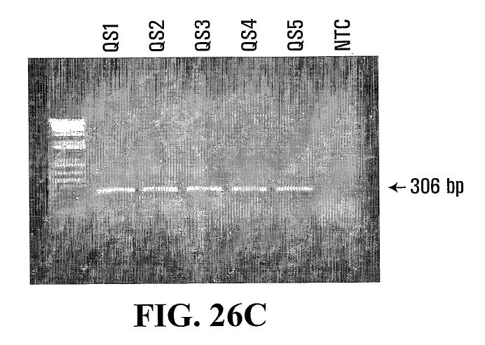

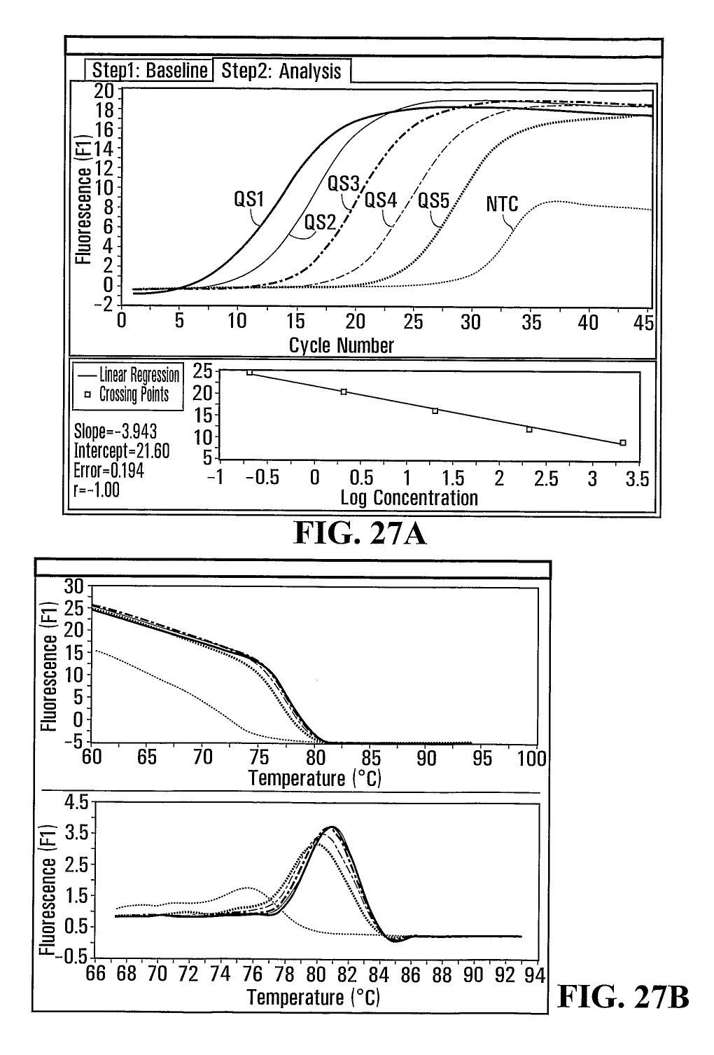

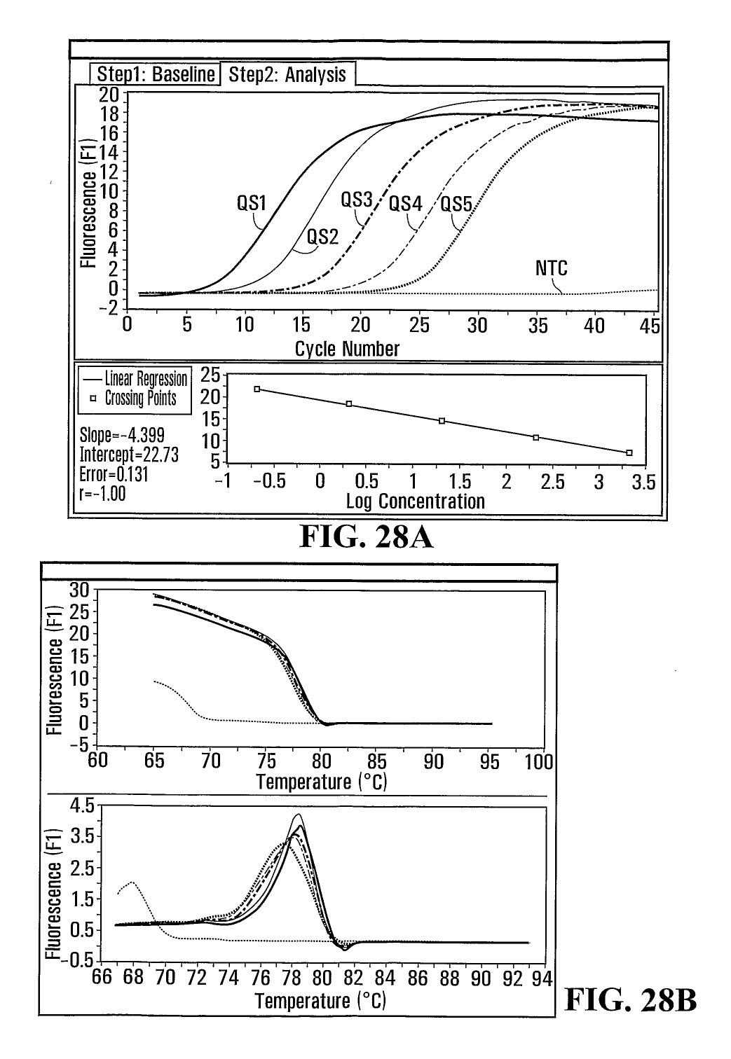

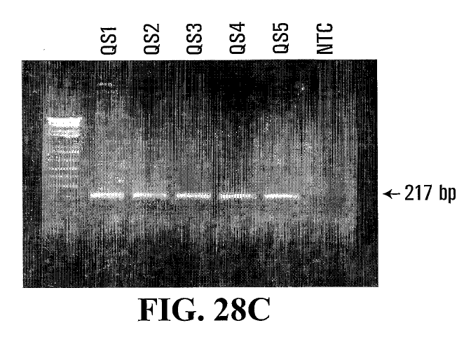

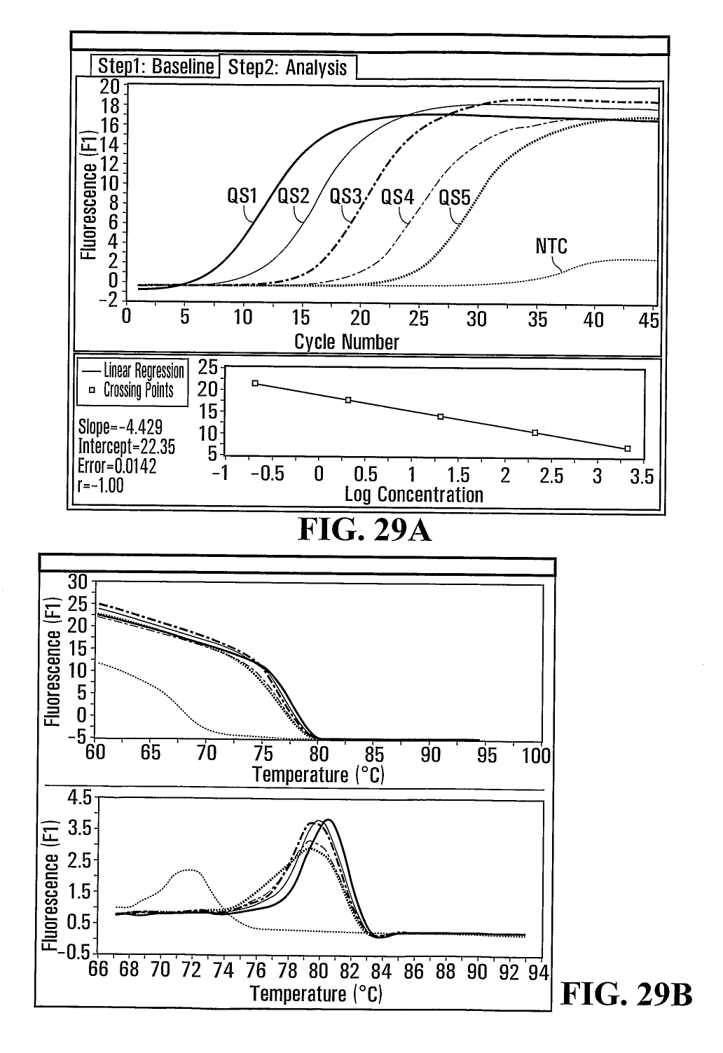

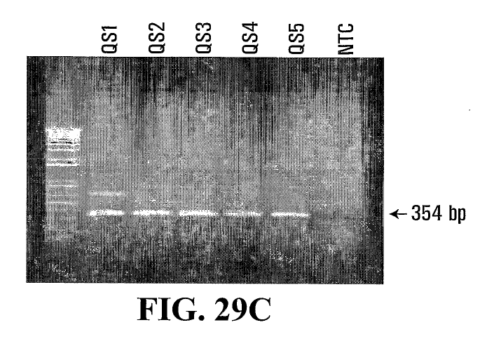

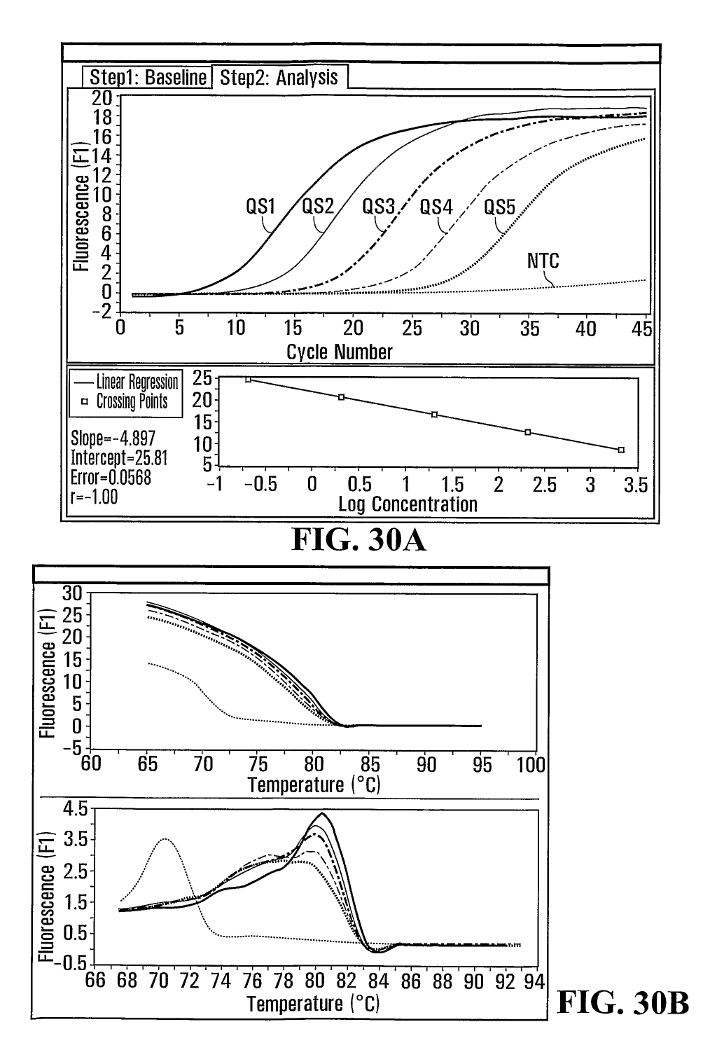

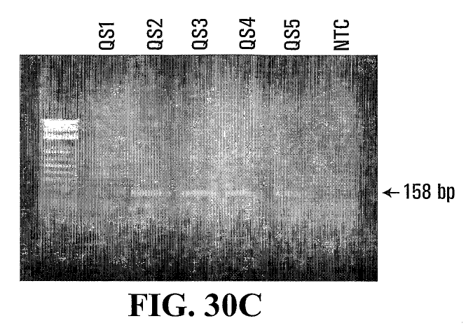

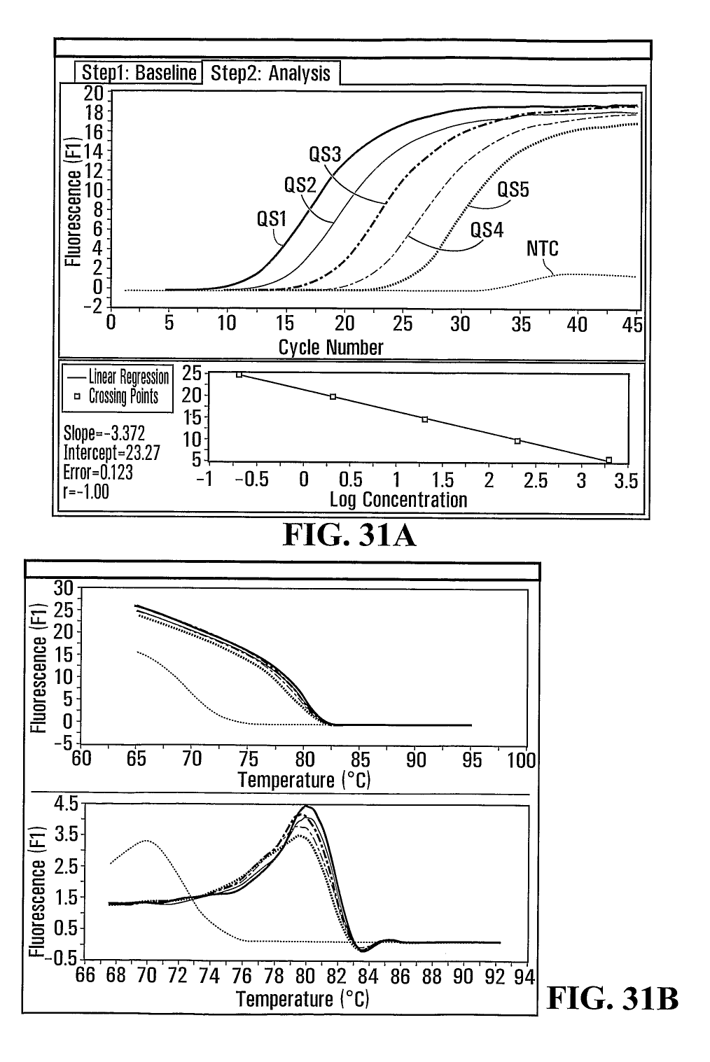

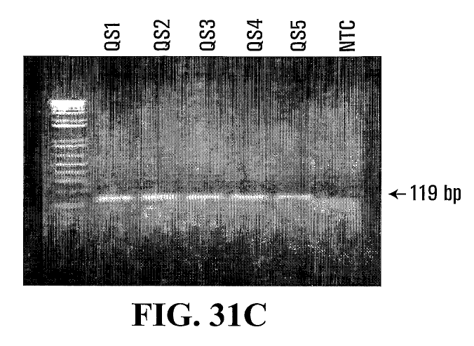

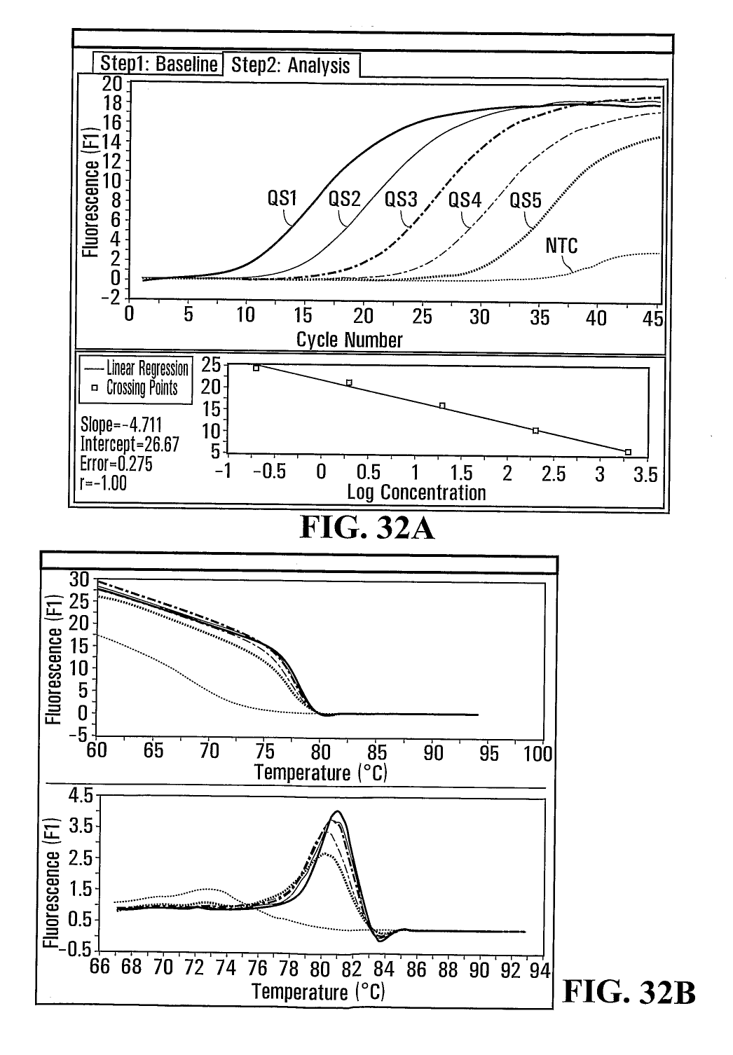

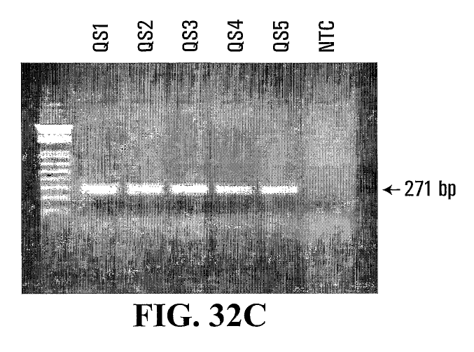

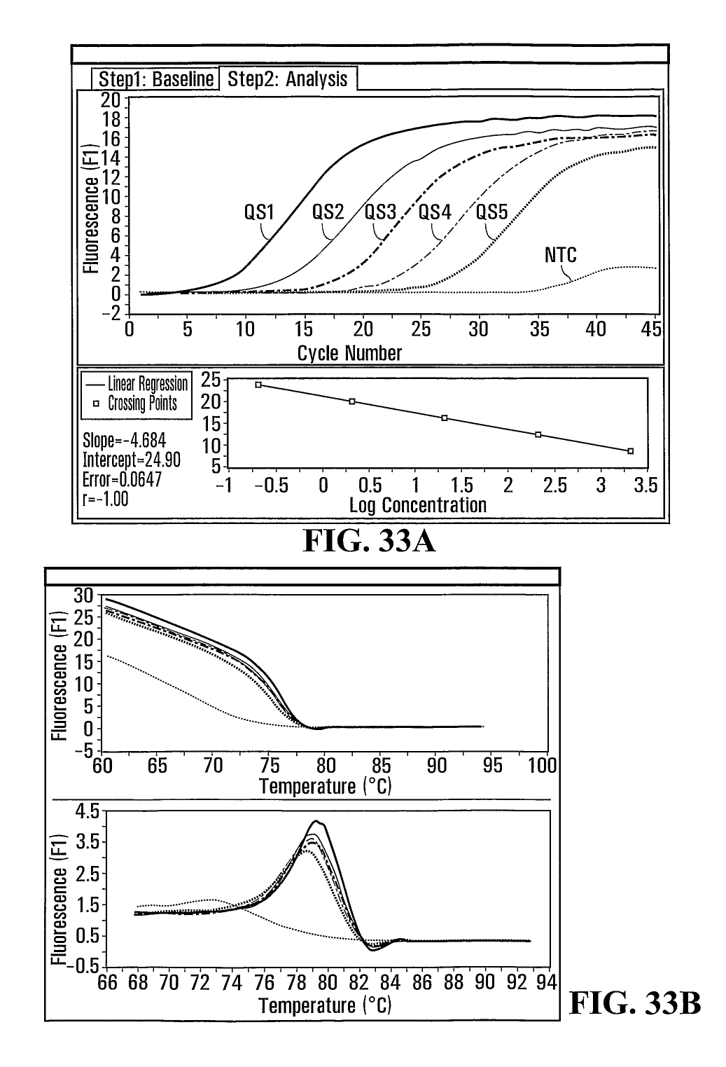

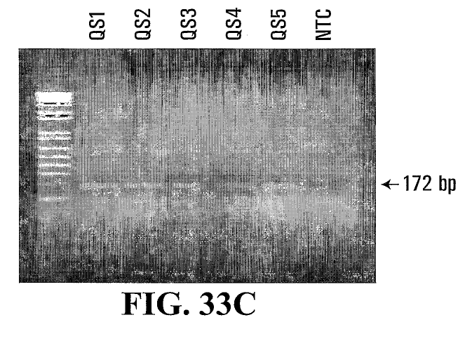

FIGS. 22-33 shows the results of real-time PCR experiments using primer sets 9 to 20, respectively. For each of FIGS. 22-33, Panel A shows the amplification curve and RNA standard curve for various concentrations of in vitro transcribed RNA template, Panel B shows the melting curves and Panel C shows the agarose gel visualization of the PCR product.

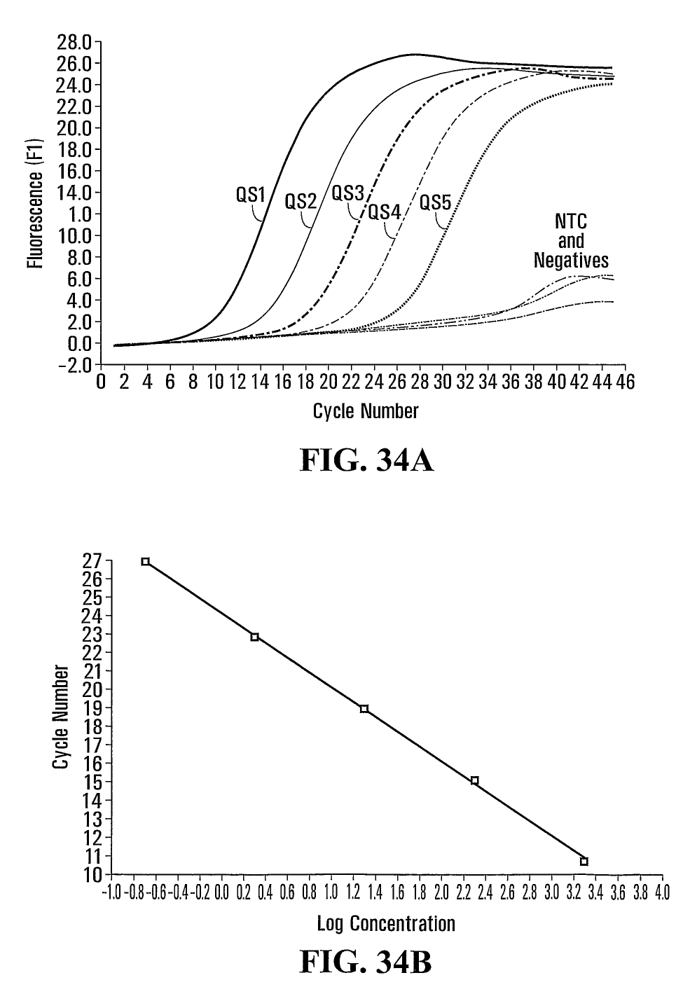

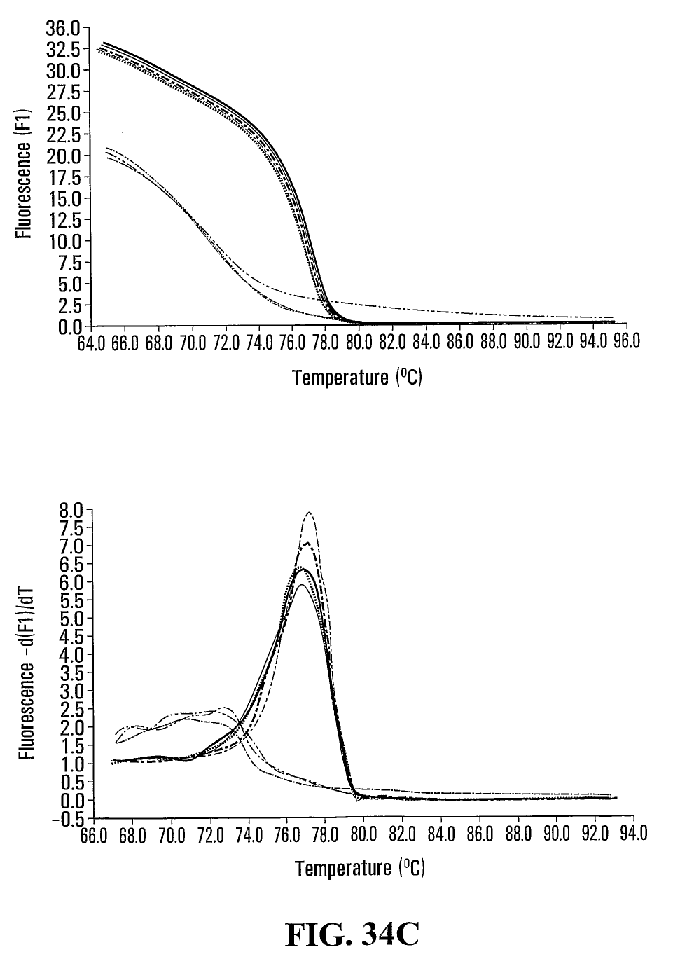

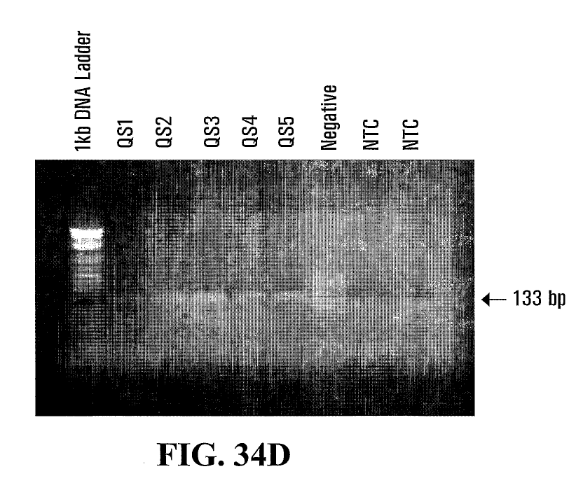

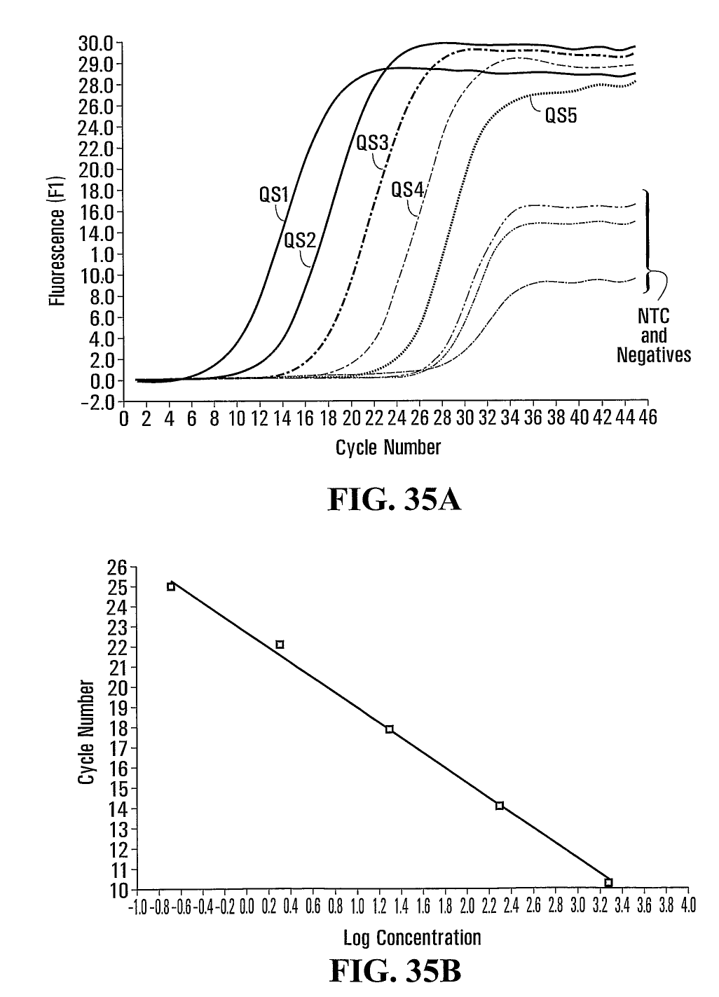

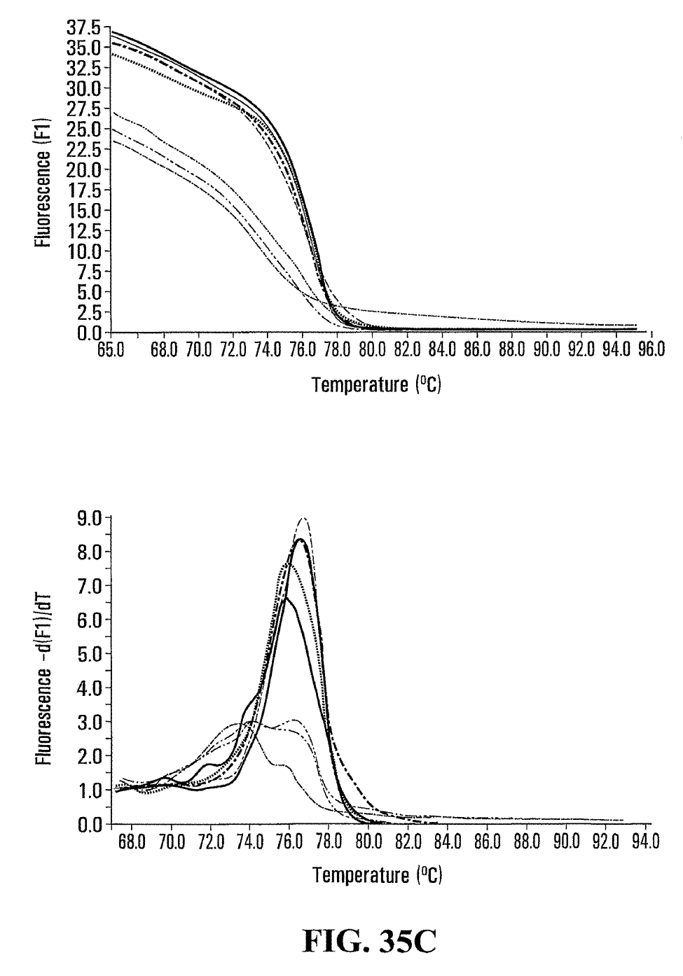

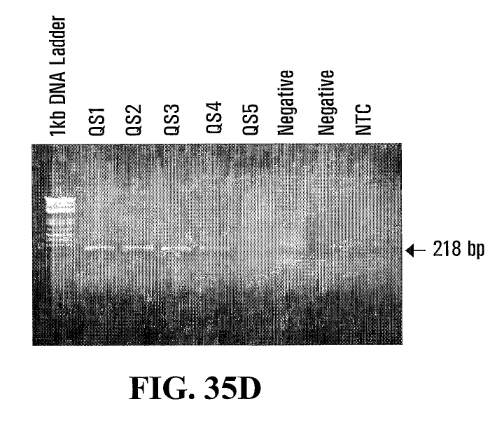

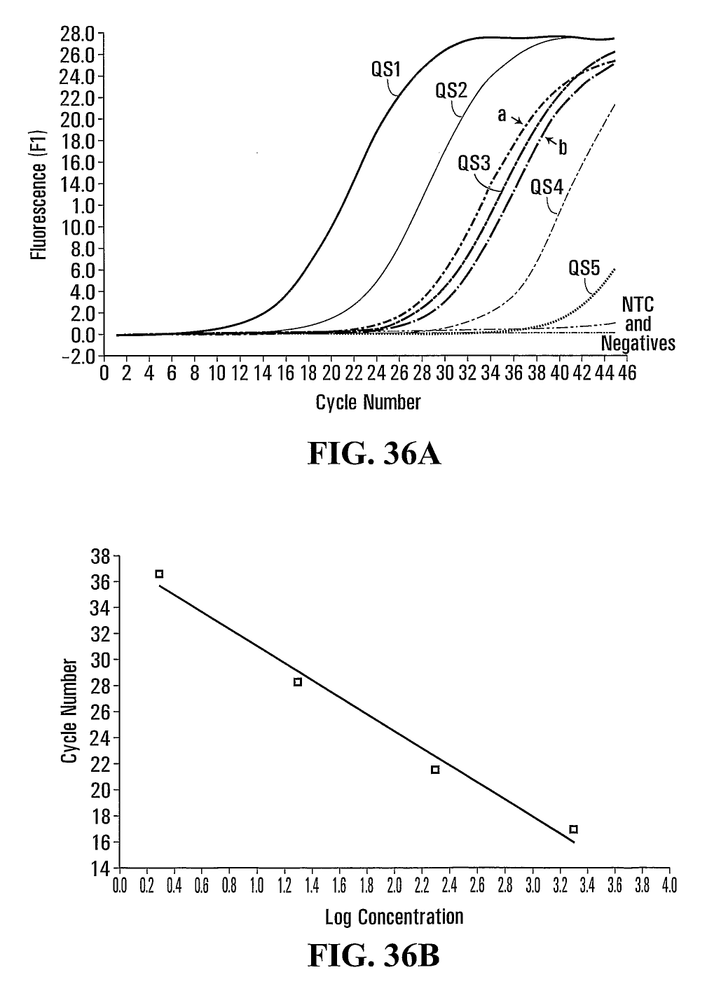

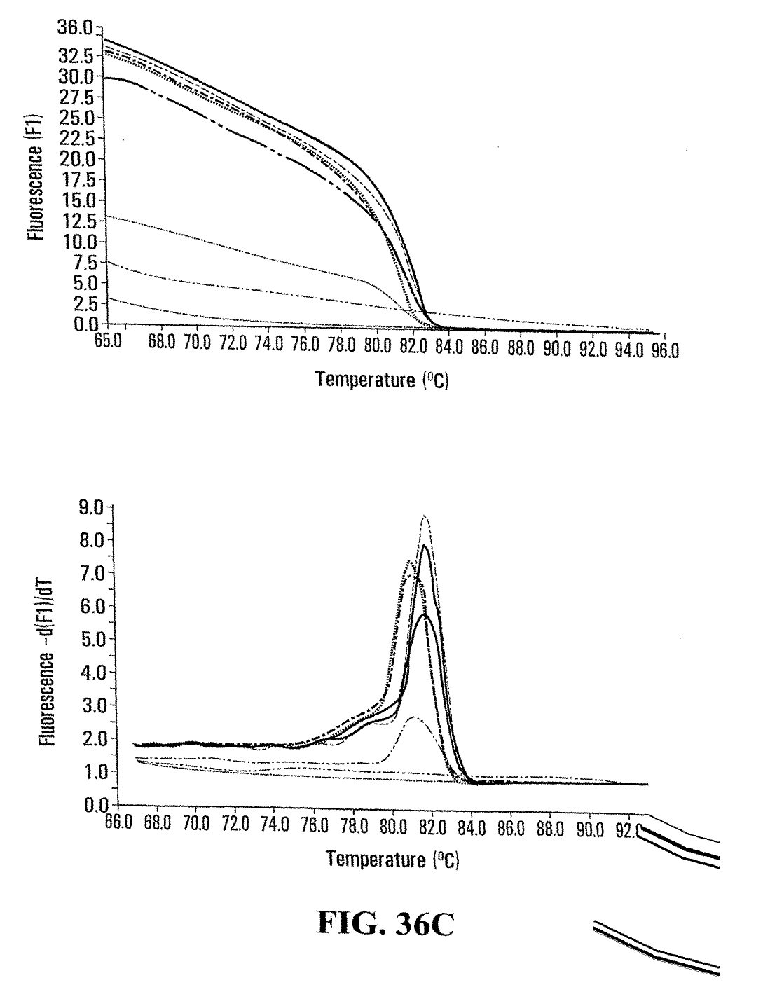

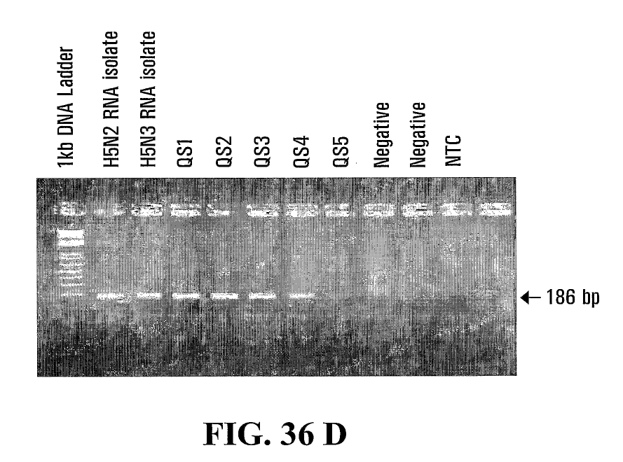

Similar real time PCR experiments were performed using the following primer sets directed against the HA gene of H5N1 subtype: (21) gisAFH5N1H9F and gisAFH5N1H11cR; and (22) gisAFH5N1H13F and gisAFH5N1H16cR; and the HA gene of H5 subtype (H5N1, H5N2 and H5N3): (23) gisAFH5H1aF and gisAFH5H2aR. The results obtained with sets 21 and 22 are shown in FIGS. 34 and 35, respectively, and the results obtained with set 23 are shown in FIG. 36, where a. and b. are the results for H5N2 and H5N3 RNA isolates, respectively. For these figures, Panel A shows the amplification curve for various concentrations of template. Template used was in vitro transcribed RNA for H5N1 and H5 subtypes and RNA extracted from field samples of H5N2 and H5N3. Panel B shows the RNA standard curve and Panel C shows the melting curves and Panel D shows the agarose gel visualization of the PCR product. The standard curves were generated using the LightCycler software based on QS1 to QS5, which are quantified amounts of in vitro transcribed RNA standards.

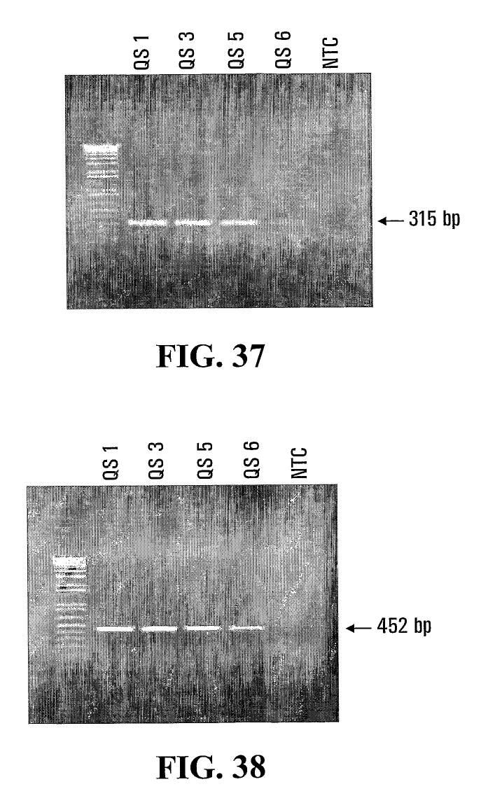

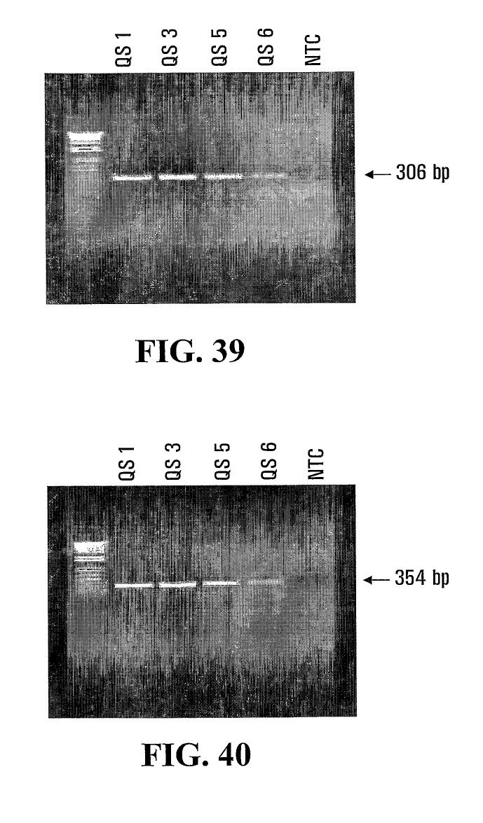

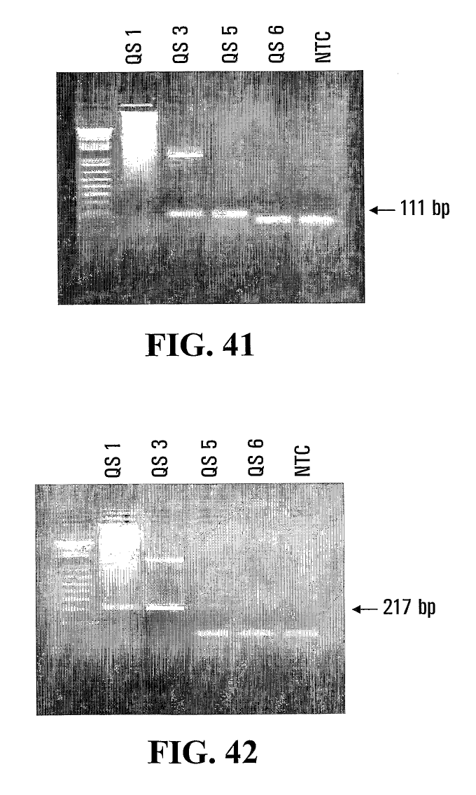

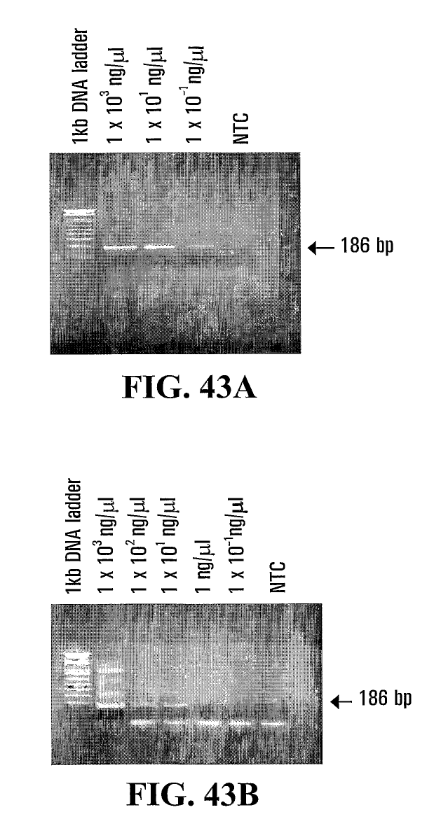

Next, one- or two-step RT-PCR reactions were performed using selected H5N1 NA primer sets (sets 10, 11, 13 and 16 using one-step; sets 12 and 15 using two-step) (FIGS. 37 to 42, respectively) and one- and two-step RT-PCR reactions were performed using the H5 HA primer set 23 (FIGS. 43 A and B, respectively), as described above. The results were visualized using 1.5% agarose gel stained with ethidium bromide, as shown in FIGS. 37 to 43.

### Example 6

### Real Time PCR Method Using the Taqman™ Probe System

A master mix was prepared in 1.5 ml reaction tubes on ice by adding in the following order: 1.3 μl of 50 mM Mn(OAc)₂, 8 μl of 1 μl probe, 1 μl of F Primer, 11 of R Primer and 7.5 μl of LightCycler RNA Master Hybridization Probes. The master mix was gently mixed and 18 μl transferred to precooled glass capillaries. 2 μl of RNA template was added and the capillaries were centrifuged at 700×g for 5 seconds. The capillaries were placed in the rotor of a LightCycler™ instrument and cycled using the programs set out in Tables 5 to 8, with 45 cycles for Program 7.

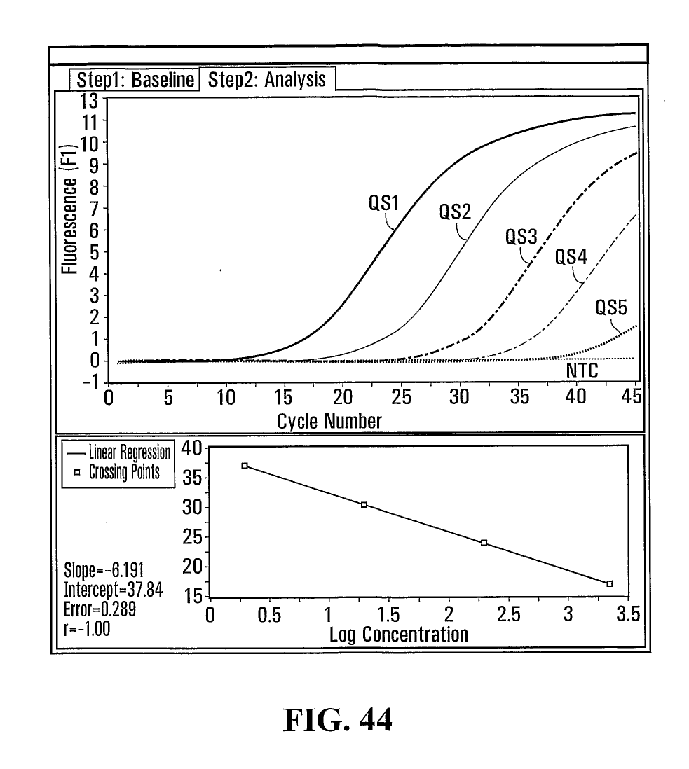

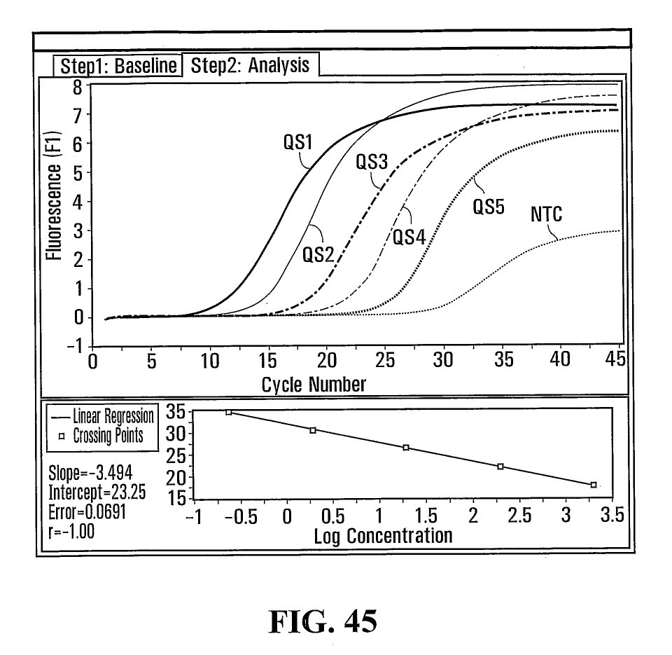

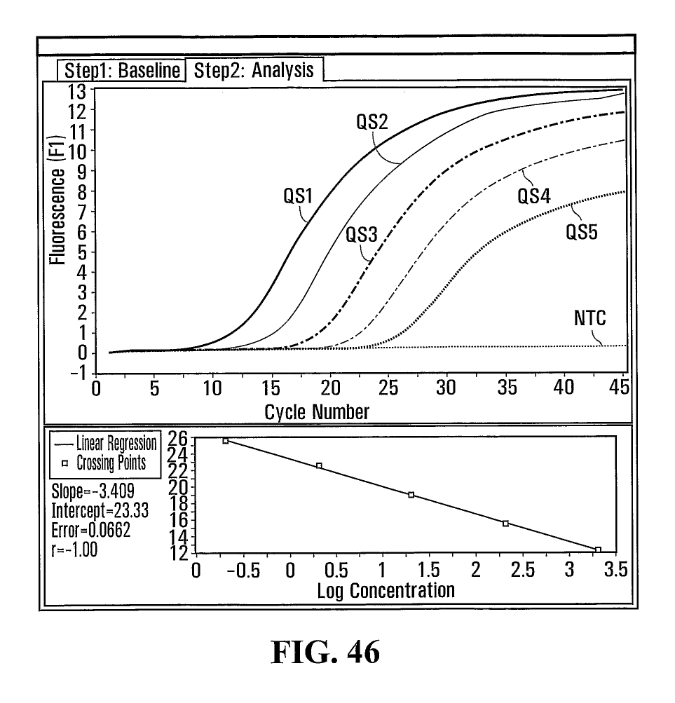

Real time PCR was performed using the Taqman™ (Roche) system of detection. To detect the HA gene of subtype H5, the following pair of amplification primers was used: (24) gisAFH5H1aF and gisAFH5HTqR, along with the Taqman™ probe gisAFH5HTMP. To detect the HA gene of subtype H5N1, the following pairs of amplification primers were used: (25) gisAFH5N1HTqF1 and gisAFH5N1H11cR; and (26) gisAFH5N1H9F and gisAFH5N1HTqR1, along with Taqman™ probes gisAFH5N1HTMP2 and gisAFH5N1HTMP1, respectively. The Taqman™ probes were labelled with the reporter fluorophore 6-carboxy fluorescein amidite (6-Fam) at the 5′ end and with the quencher tetramethyl rhodamine (TAMRA) at the 3′ end. The sequences of the Taqman™ probes are shown in Table 10. Amplification curve results are shown in FIG. 44 for H5 primer set 24 and FIGS. 45 and 46 for H5N1 primer sets 25 and 26.

TABLE 10
Taqman™ Primers

| | | Start | |
|---|---|---|---|
| SEQ ID | | Nucleotide | Sequence |
| NO: | Primer | (gene) | (5′ to 3′) |
| 115 | gisAFH5HTMP | 79 (HA) | 6Fam- AAC TCG ACA |
| | | | GAG CAG GTT GAG |
| | | | ACA ATA A- TAMRA |
| 116 | gisAFH5N1HTMP | 1840 (HA) | 6Fam- TAT GAA AAG |
| | | | TGA ATT GGA ATA |
| | | | TGG TAA CTG- TAMRA |
| 117 | gisAFH5N1HTMP2 | 854 (HA) | 6Fam- TGG AAT ATG |
| | | | GTA ACT GCA ACA |
| | | | CCA AGT GTC A- |
| | | | TAMRA |
| 119 | gisAFH5N1NTMP1 | 492 (NA) | 6Fam- AGT GCT TGC |
| | | | CAT GAT GGC ACC |
| | | | AGT- TAMRA |
| 119 | gisAFH5N1NTMP1 | 492 (NA) | JOE- AGT GCT TGC |
| | Joe | | CAT GAT GGC ACC |
| | | | AGT-TAMRA |
| 119 | gisAFH5N1NTMP1 | 492 (NA) | JOE- AGT GCT TGC |
| | Joe(BHQ) | | CAT GAT GGC ACC |
| | | | AGT-BHQ-1 |

### Example 7

### DNA Microarray Using Primers

Particular primers of the invention were used in a DNA microarray (Attogenix, Singapore) to detect RNA from H5 and H5N1 isolates. Briefly, various HA and NA primers, specifically H5 (gisAFH5H1aF and gisAFH5H2aR), H5N1 (gisAFH5N1H2aF and gisAFH5N1H4R), and NA H5N1 (gisAFH5N1N2aF and gisAFH5N1N3aR) were immobilized on a solid surface (GAPDH was used as a positive control for RT-PCR). The microarray was then probed with sample NA or HA transcript RNA, or both, and binding of the probe to the primer in each spot in the microarray was detected using Sybr Green fluorescent probe to detect double-stranded nucleic acid. Results are shown in Table 11. As can been seen, the NA transcript was detectable at lower concentrations than the HA transcript, indicating that the NA primers used are more sensitive than the particular HA primers used.

TABLE 11
Detection of NA and HA Gene Transcripts using DNA Microarray

Ct Value

| | Primer | Primer | Primer | |
|---|---|---|---|---|
| Sample | HA H5 | HA H5N1 | NA H5N1 | GAPDH |
| negative | nil | nil | nil | 36.15 |
| NA RNA | nil | nil | 20.35 | 34.36 |
| (20 ng) | | | | |
| HA RNA | nil | nil | nil | 35.71 |
| (20 ng) | | | | |
| NA + HA | nil | 41.93 | 20.37 | 34.80 |
| RNA (10 | | | | |
| ng each) | | | | |
| HA RNA | 41.68 | 19.82 | nil | 34.80 |
| (200 ng) | | | | |
| HA RNA | 38.95 | 20.97 | 19.74 | 33.14 |
| (100 ng) + | | | | |
| NA RNA | | | | |
| (10 ng) | | | | |

### Example 8

### Real Time RT-PCR Method Using the Taqman™ Probe System

The PCR reaction mixture was prepared in 1.5 ml reaction tubes on ice by adding in the following order: 12.5 μl of 2× QuantiTect™ Probe RT-PCR Master Mix, 1.25 μl of 10 μM forward primer, 1.25 μl of 10 μM reverse primer, 0.5 μl of 10 μM probe, 0.25 μl of QuantiTect™ RT Mix. The reaction mixture was mixed gently and 2-5 μl of RNA template was added and the volume was made up to 25 μl using RNase-free water. The reaction tubes were placed in the rotor of an ABI™ cycler instrument and cycled using the following program: 50° C. for 30 minutes (reverse transcription); 95° C. for 15 minutes (hot start); 45 cycles of 94° C. for 15 seconds and 60° C. for 60 seconds. Data acquisition step was performed during the combined annealing/extension step.

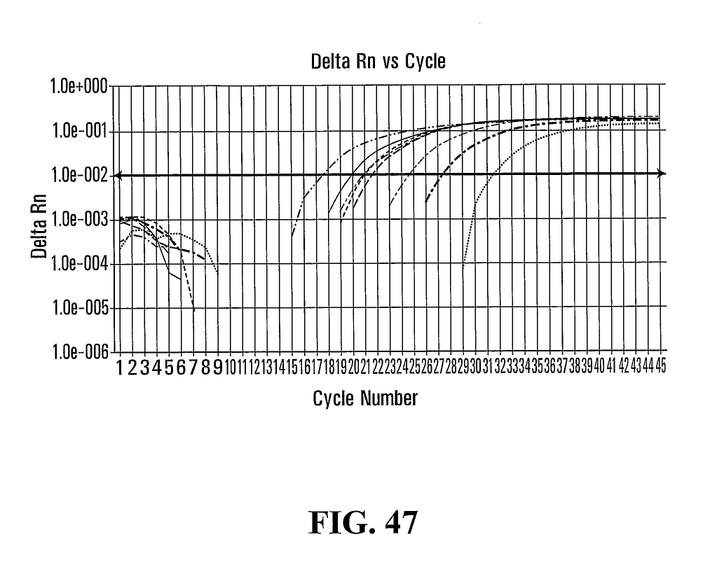

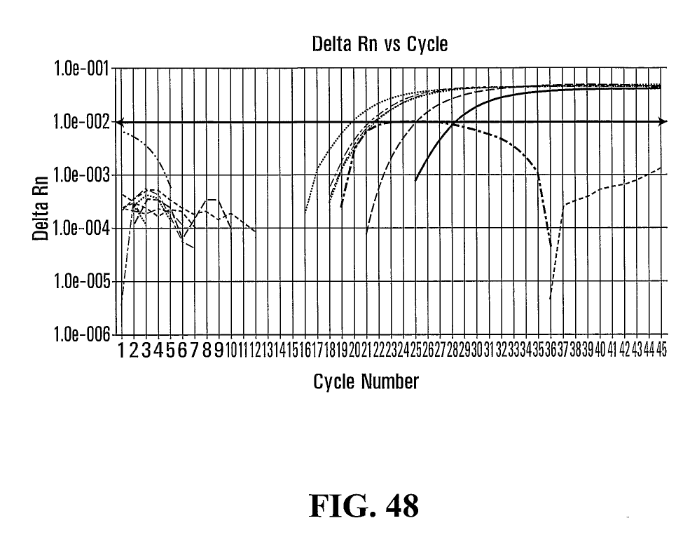

Real time PCR was performed using the Taqman™ (Roche) system of detection. To detect the HA gene of subtype H5N1, the following pairs of amplification primers were used: (25) gisAFH5N1HTqF1 and gisAFH5N1H11cR; and (26) gisAFH5N1H9F and gisAFH5N1HTqR1, along with Taqman™ probes gisAFH5N1HTMP2 for primer set 25 and gisAFH5N1HTMP1 for primer set 26. These Taqman™ probes are described above in Example 6. FIGS. 47 and 48 contain Delta Rn curves for results obtained using primer sets 25 and 26. The Delta Rn (delta reaction) curve is similar to the fluorescence versus cycle number plot in the LightCycler system.

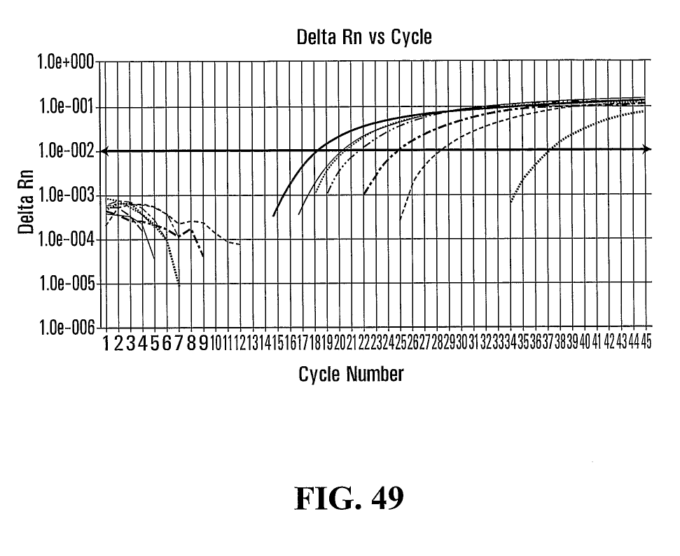

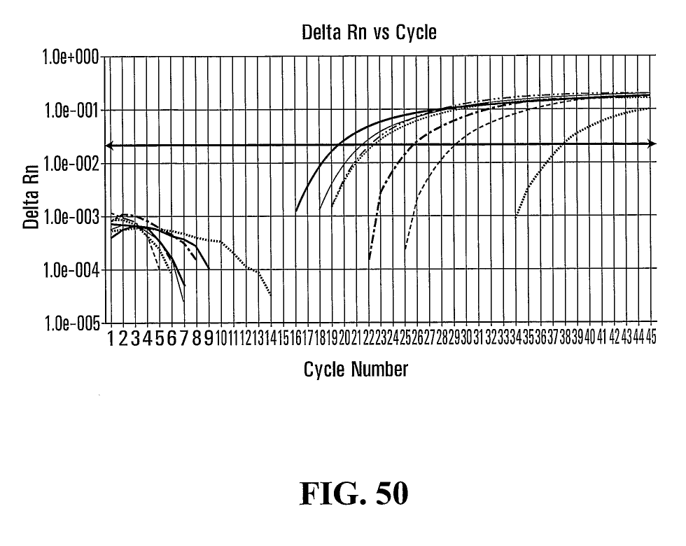

To detect the NA gene of subtype H5N1, the following pair of amplification primers was used: (27) gisAFH5N1N1aF and gisAFH5N1NTqR1, along with Taqman™ probe gisAFH5N1NTMP1 (sequence shown in Table 10 above) or gisAFH5N1NTMP1Joe. gisAFH5N1NTMP1Joe has the same nucleotide sequence as gisAFH5N1NTMP1, but is labelled with the reporter molecule JOE™ (Taqman) at the 5′ end rather than 6-Fam. Additionally, the Taqman™ probes may be labelled with the quencher molecule Black Hole Quencher (BHQ) at the 3′ end. FIGS. 49 and 50 contain Delta Rn curves for results obtained using primer set 27, detected using Taqman™ probe gisAFH5N1NTMP1 or gisAFH5N1NTMP1Joe, respectively.

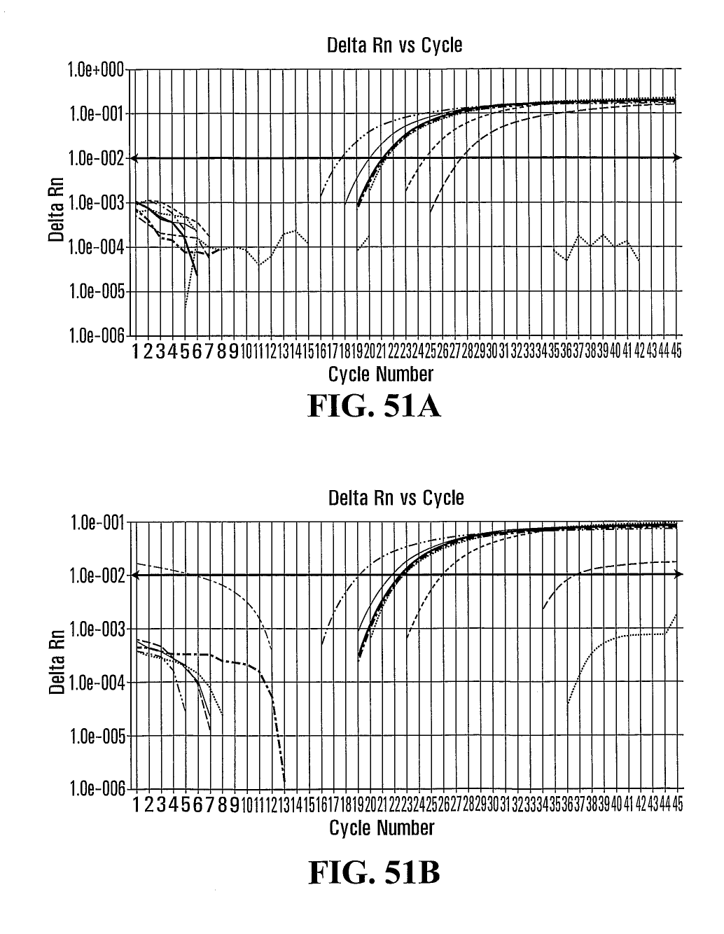

The above experiment was also performed as a multiplex reaction, in which two different primer sets and Taqman™ probes were used in a single reaction mixture. The reaction mix was prepared as described above, with the primer sets 25 and 27 used, with probes gisAFH5N1HTMP2 and gisAFH5N1NTMP1Joe. The Delta Rn curves are shown in FIG. 51.

### Example 9

### Real Time RT-PCR Method Using the Taqman™ Probe System

The following protocol may be used with an ABI 7500 or 7900 cycler machine with QuantiTect™ Probe One-Step RT-PCR system (Qiagen).

In a sterile 1.5 ml reaction tube on ice, prepare the PCR Mix for one 25 μl reaction by adding the following components in the following order:

TABLE 12
Reagents for RT-PCR protocol using Taqman ™ System

| Component | Volume |
|---|---|
| 2x QuantiTect Probe RT-PCR Master Mix | 12.5 μl |
| Forward primer (10 μM) | 1.25 μl |
| Reverse primer (10 μM) | 1.25 μl |
| Probe (10 μM) | 0.5 μl |
| QuantiTect RT Mix | 0.25 μl |
| RNase-free water | Variable (to make |
| | up final volume |
| | to 25 μl) |

To prepare the PCR Master Mix for more than one reaction, multiply the amount in the “Volume” column above by the number of reactions. Note: always prepare at least 1 to 2 more reactions than actual number of reactions. Mix gently, aliquot required amount into each sterile PCR tubes or plates and add Template RNA (2.0 μl for RNA controls; up to 5.0 μl for RNA samples).

Program the ABI cycler machine according to the program below:

TABLE 12
RT-PCR Real Time Program

| Step | Parameters | Cycles |
|---|---|---|
| Reverse transcription | 50° C. for 30 min | 1 |
| Hot start | 95° C. for 15 min | 1 |
| 2-step cycling: | | 45 |
| Denaturation | 94° C. for 15 sec | |
| Annealing/Extension | 60° C. for 60 sec | |

Data acquisition step is performed during the combined annealing/extension step. View results and check for amplification curves and CT values.

### Example 10

### Real Time RT-PCR Method Using the Taqman™ Probe System for Multiplex Reactions

The following protocol may be used with an ABI 7500 or 7900 cycler machine with QuantiTect™ Probe One-Step RT-PCR system (Qiagen).

In a sterile 1.5 ml reaction tube on ice, prepare the PCR Mix for one 25 μl reaction by adding the following components in the following order:

TABLE 12
Reagents for RT-PCR protocol using Taqman ™ System

| Component | Volume | |
|---|---|---|
| 2x QuantiTect Probe RT-PCR Master Mix | 12.5 | μl |
| Forward primer 1 (10 μM) | 1.25 | μl |
| Reverse primer 1 (10 μM) | 1.25 | μl |
| Probe 1 (10 μM) | 0.5 | μl |
| Forward primer 2 (10 μM) | 1.25 | μl |
| Reverse primer 2 (10 μM) | 1.25 | μl |
| Probe 2 (10 μM) | 0.5 | μl |
| QuantiTect RT Mix | 0.25 | μl |

| | |
|---|---|
| RNase-free water | Variable (to make |
| | up final volume |
| | to 25 μl) |

To prepare the PCR Master Mix for more than one reaction, multiply the amount in the “Volume” column above by the number of reactions. Note: always prepare at least 1 to 2 more reactions than actual number of reactions. Mix gently, aliquot required amount into each sterile PCR tubes or plates and add Template RNA (2.0 μl for RNA controls; up to 5.0 μl for RNA samples).

Program the ABI cycler machine according to the program below:

TABLE 12
RT-PCR Real Time Program

| Step | Parameters | Cycles |
|---|---|---|
| Reverse transcription | 50° C. for 30 min | 1 |
| Hot start | 95° C. for 15 min | 1 |
| 2-step cycling: | | 45 |
| Denaturation | 94° C. for 15 sec | |
| Annealing/Extension | 60° C. for 60 sec | |

Data acquisition step is performed during the combined annealing/extension step. View results and check for amplification curves, CT values and melt curves.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. All technical and scientific terms used herein have the same meaning as commonly understood by one of ordinary skill in the art of this invention, unless defined otherwise.

As can be understood by one skilled in the art, many modifications to the exemplary embodiments described herein are possible. The invention, rather, is intended to encompass all such modification within its scope, as defined by the claims.

1. A primer comprising one of the following: (i) the sequence of SEQ ID NO:118; and (ii) a target annealing sequence and a non-influenza A virus sequence, wherein the target annealing sequence comprises the sequence of SEQ ID NO:118.

2. The primer of claim 1 wherein (i) the primer consists essentially of the sequence of SEQ ID NO:118; or (ii) the target annealing sequence consists essentially of the sequence of SEQ ID NO:118.

3. The primer of claim 1 wherein (i) the primer is the sequence of SEQ ID NO:118; or (ii) the target annealing sequence is the sequence of SEQ ID NO:118.

4. (canceled)

5. (canceled)

6. (canceled)

7. The primer of claim 1 further comprising a label.

8. The primer of claim 7 wherein the label is a fluorescent label, a chemiluminescent label, a coloured dye label, a radioactive label, a radiopaque label, a protein including an enzyme, a peptide or a ligand.

9. A method for detecting influenza A virus subtype H5N1 in a sample comprising:
amplifying DNA reverse transcribed from RNA obtained from the sample using a primer as defined in claim 1;
and detecting a product of amplification, wherein the presence of the product of amplification indicates the presence of an avian influenza virus subtype H5N1 in the sample.

10. The method of claim 9 wherein each primer consists essentially of the sequence of SEQ ID NO:118 or each primer is the sequence of SEQ ID NO:118.

11. (canceled)

12. The method of claim 9, wherein said amplifying comprises using a primer set, the primer set comprising
one or more reverse primers each comprising a sequence of any one of SEQ ID NO:94 to SEQ ID NO:111 and SEQ ID NO:118, one of said one or more reverse primers comprising the sequence of SEQ ID NO:118 and one or more forward primers each comprising a sequence of any one of SEQ ID NO:72 to SEQ ID NO:93;
wherein the presence of the product of amplification indicates the presence of an avian influenza virus subtype subtype H5N1 in the sample.

13. The method of claim 9 further comprising the step of reverse transcribing RNA obtained from the biological sample using one or more reverse primers each comprising a sequence of any of SEQ ID NO:94 to SEQ ID NO:111 and SEQ ID NO:118, one of said one or more reverse primers comprising the sequence of SEQ ID NO:118.

14. The method of claim 13 wherein said amplifying and said reverse transcribing are performed in a single reaction mixture.

15. The method of claim 12 wherein said one or more reverse primers each has a sequence of any one of SEQ ID NO:94 to SEQ ID NO:111 and SEQ ID NO:118.

16. The method of claim 12 wherein said one or more forward primers each has the sequence of any one of SEQ ID NO:72 to SEQ ID NO:93.

17. The method of claim 9 wherein the step of amplifying comprises amplifying by PCR amplification.

18. The method of claim 17 wherein the step of amplifying includes a hot start.

19. The method of claim 17 wherein the detecting step comprises detecting by an agarose or acrylamide gel.

20. The method of claim 9 wherein the detecting step comprises detecting by real time PCR.

21. The method of claim 20 wherein said detecting by real time PCR comprises detecting with a detection probe having a fluorophore at the 5′ end and a quenching molecule at the 3′ end.

22. A method of detecting influenza A virus subtype H5N1 in a sample comprising:
contacting the sample with a primer as defined in claim 1 immobilized on a support under conditions suitable for hybridizing the primer and the sample; and
detecting hybridization of the primer and the sample.

23. The method of claim 22 wherein the primer consists essentially of the sequence of SEQ ID NO:118 or each primer is the sequence of SEQ ID NO:118.

24. (canceled)

25. A method of detecting influenza A virus subtype H5N1 in a sample comprising:
contacting the sample with a nucleic acid microarray, the nucleic acid microarray comprising one or more primers, one of said one or more primers being a primer as defined in claim 1, under conditions suitable for hybridizing the one or more primers and the sample; and
detecting hybridization of the one or more primers and the sample.

26. The method of claim 25 wherein each of said one or more primers consists essentially of any one of the sequences of SEQ ID NO:1 to SEQ ID NO:114 and SEQ ID NO:118 or each of said one or more primers is any one of the sequences of SEQ ID NO:1 to SEQ ID NO:114 and SEQ ID NO:118.

27. (canceled)

28. A nucleic acid microarray comprising a primer as defined in claim 1.

29. The nucleic acid microarray of claim 28 wherein the primer consists essentially of the sequence of SEQ ID NO:118 or the primer is the sequence of SEQ ID NO:118.

30. (canceled)

31. A kit comprising a primer as defined in claim 1 and instructions for detecting influenza A virus subtype H5 or H5N1 in a sample.

32.-62. (canceled)


SEQUENCE LISTING (119 sequences)

SEQ ID NO: 1 - DNA, 23 bp, artificial
  artifical primer
atttgcattggttaccatgcaaa

SEQ ID NO: 2 - DNA, 23 bp, artificial
  artifical primer
atttgcattggttatcatgcaaa

SEQ ID NO: 3 - DNA, 23 bp, artificial
  artifical primer
atctgcattggttatcatgcaaa

SEQ ID NO: 4 - DNA, 23 bp, artificial
  artifical primer
atctgcatcggttatcatgcaaa

SEQ ID NO: 5 - DNA, 23 bp, artificial
  artifical primer
atttgcatcggttaccatgcaaa

SEQ ID NO: 6 - DNA, 23 bp, artificial
  artifical primer
atttgcattggtcatcatgcaaa

SEQ ID NO: 7 - DNA, 23 bp, artificial
  artifical primer
gctggatggctcctcggaaaccc

SEQ ID NO: 8 - DNA, 23 bp, artificial
  artifical primer
gctggatggctccttggaaatcc

SEQ ID NO: 9 - DNA, 23 bp, artificial
  artifical primer
gctggatggctcctcggaaatcc

SEQ ID NO: 10 - DNA, 23 bp, artificial
  artifical primer
gctggatggcttcttgggaaccc

SEQ ID NO: 11 - DNA, 23 bp, artificial
  artifical primer
gctggatggctcctgggaaaccc

SEQ ID NO: 12 - DNA, 23 bp, artificial
  artifical primer
gctggatggcttcttggaaaccc

SEQ ID NO: 13 - DNA, 23 bp, artificial
  artifical primer
gccggatggcttttggggaatcc

SEQ ID NO: 14 - DNA, 23 bp, artificial
  artifical primer
gctgggtggcttcttggaaaccc

SEQ ID NO: 15 - DNA, 23 bp, artificial
  artifical primer
gctgggtggcttcttggaaatcc

SEQ ID NO: 16 - DNA, 23 bp, artificial
  artifical primer
gctggatggcttctcggaaatcc

SEQ ID NO: 17 - DNA, 23 bp, artificial
  artifical primer
gctgggtggctcctcggaaaccc

SEQ ID NO: 18 - DNA, 23 bp, artificial
  artifical primer
gccggacttgttttggggaatcc

SEQ ID NO: 19 - DNA, 23 bp, artificial
  artifical primer
cagatttgcattggttaccatgc

SEQ ID NO: 20 - DNA, 23 bp, artificial
  artifical primer
gttgacacaataatggaaaagaa

SEQ ID NO: 21 - DNA, 23 bp, artificial
  artifical primer
gttgatacaataatggaaaagaa

SEQ ID NO: 22 - DNA, 23 bp, artificial
  artifical primer
gttgacacaataatggagaagaa

SEQ ID NO: 23 - DNA, 23 bp, artificial
  artifical primer
gctggatggctcctcggaaaccc

SEQ ID NO: 24 - DNA, 22 bp, artificial
  artifical primer
actatgaagaactgaaacacct

SEQ ID NO: 25 - DNA, 22 bp, artificial
  artifical primer
actatgaagaattgaaacacct

SEQ ID NO: 26 - DNA, 23 bp, artificial
  artifical primer
gagagtaatggaaatttcattgc

SEQ ID NO: 27 - DNA, 23 bp, artificial
  artifical primer
gagagtaatggaaattttattgc

SEQ ID NO: 28 - DNA, 24 bp, artificial
  artifical primer
tgcatacaaaattgtcaagaaagg

SEQ ID NO: 29 - DNA, 23 bp, artificial
  artifical primer
atgccaytccacaayatacaccc

SEQ ID NO: 30 - DNA, 23 bp, artificial
  artifical primer
atgccattccacaatatacaccc

SEQ ID NO: 31 - DNA, 22 bp, artificial
  artifical primer
cagcttagggataatgcaaagg

SEQ ID NO: 32 - DNA, 23 bp, artificial
  artifical primer
gggtttccgaggagccatccagc

SEQ ID NO: 33 - DNA, 23 bp, artificial
  artifical primer
ggatttccaaggagccatccagc

SEQ ID NO: 34 - DNA, 23 bp, artificial
  artifical primer
ggatttccgaggagccatccagc

SEQ ID NO: 35 - DNA, 23 bp, artificial
  artifical primer
gggttcccaagaagccatccagc

SEQ ID NO: 36 - DNA, 23 bp, artificial
  artifical primer
gggtttcccagaagccatccagc

SEQ ID NO: 37 - DNA, 23 bp, artificial
  artifical primer
gggtttccaagaagccatccagc

SEQ ID NO: 38 - DNA, 23 bp, artificial
  artifical primer
ggattccccaaaagccatccggc

SEQ ID NO: 39 - DNA, 23 bp, artificial
  artifical primer
gggtttccaagaagccacccagc

SEQ ID NO: 40 - DNA, 23 bp, artificial
  artifical primer
ggatttccaagaagccacccagc

SEQ ID NO: 41 - DNA, 23 bp, artificial
  artifical primer
ggatttccgagaagccatccagc

SEQ ID NO: 42 - DNA, 23 bp, artificial
  artifical primer
gggtttccgaggagccacccagc

SEQ ID NO: 43 - DNA, 23 bp, artificial
  artifical primer
ggattccccaaaacaagtccggc

SEQ ID NO: 44 - DNA, 23 bp, artificial
  artifical primer
gggtgtatgttgtggaatggcat

SEQ ID NO: 45 - DNA, 23 bp, artificial
  artifical primer
gggtgtatattgtggaagggcat

SEQ ID NO: 46 - DNA, 23 bp, artificial
  artifical primer
ggatgtatattgtggaatggcat

SEQ ID NO: 47 - DNA, 23 bp, artificial
  artifical primer
ggatgaacattgtggaaaggcat

SEQ ID NO: 48 - DNA, 23 bp, artificial
  artifical primer
ggatggacattatggaaaggcat

SEQ ID NO: 49 - DNA, 23 bp, artificial
  artifical primer
gggtgtatgttatggaatggcat

SEQ ID NO: 50 - DNA, 23 bp, artificial
  artifical primer
ggatgtacattgtgaaaaggcat

SEQ ID NO: 51 - DNA, 23 bp, artificial
  artifical primer
ggatgaacattgtggaagggcat

SEQ ID NO: 52 - DNA, 23 bp, artificial
  artifical primer
ggatgtatattgtggaacggcat

SEQ ID NO: 53 - DNA, 23 bp, artificial
  artifical primer
ggatgtatgttgtggaatggcat

SEQ ID NO: 54 - DNA, 23 bp, artificial
  artifical primer
ggatgtatgttgtggaacggcat

SEQ ID NO: 55 - DNA, 23 bp, artificial
  artifical primer
gggtgtatattgtggaatggcat

SEQ ID NO: 56 - DNA, 23 bp, artificial
  artifical primer
gggtttccgaggagccatccagc

SEQ ID NO: 57 - DNA, 22 bp, artificial
  artifical primer
aggtgtttcagttcttcatagt

SEQ ID NO: 58 - DNA, 22 bp, artificial
  artifical primer
aggtgtttcaattcttcatagt

SEQ ID NO: 59 - DNA, 23 bp, artificial
  artifical primer
gcaatgaaatttccattactctc

SEQ ID NO: 60 - DNA, 23 bp, artificial
  artifical primer
gcaataaaatttccattactctc

SEQ ID NO: 61 - DNA, 24 bp, artificial
  artifical primer
cctttcttgacaattttgtatgca

SEQ ID NO: 62 - DNA, 23 bp, artificial
  artifical primer
gggtgtatgttgtggaatggcat

SEQ ID NO: 63 - DNA, 23 bp, artificial
  artifical primer
gggtgtatattgtggagtggcat

SEQ ID NO: 64 - DNA, 23 bp, artificial
  artifical primer
gggtgtatattgtggaatggcat

SEQ ID NO: 65 - DNA, 22 bp, artificial
  artifical primer
cctttgcattatccctaagctg

SEQ ID NO: 66 - DNA, 23 bp, artificial
  artifical primer
tccaattttactccacttatttc

SEQ ID NO: 67 - DNA, 23 bp, artificial
  artifical primer
tccaattttaccccacttatttc

SEQ ID NO: 68 - DNA, 23 bp, artificial
  artifical primer
tccaattttactccacttatttt

SEQ ID NO: 69 - DNA, 23 bp, artificial
  artifical primer
tagggaactcgccactgttgaat

SEQ ID NO: 70 - DNA, 23 bp, artificial
  artifical primer
tagggaactcgccactgctgaat

SEQ ID NO: 71 - DNA, 23 bp, artificial
  artifical primer
tagggaactcgccactgtagaat

SEQ ID NO: 72 - DNA, 23 bp, artificial
  artifical primer
tttgagtctgttgcttggtcagc

SEQ ID NO: 73 - DNA, 23 bp, artificial
  artifical primer
tttgagtcggttgcttggtcggc

SEQ ID NO: 74 - DNA, 23 bp, artificial
  artifical primer
tttgagtctgttgcttggtcggc

SEQ ID NO: 75 - DNA, 23 bp, artificial
  artifical primer
gttgcttggtcagcaagtgcttg

SEQ ID NO: 76 - DNA, 23 bp, artificial
  artifical primer
gttgcttggtcggcaagtgcttg

SEQ ID NO: 77 - DNA, 23 bp, artificial
  artifical primer
gttgcttggtcggcaagcgcttg

SEQ ID NO: 78 - DNA, 23 bp, artificial
  artifical primer
gaaatacaatggcataataacag

SEQ ID NO: 79 - DNA, 23 bp, artificial
  artifical primer
gaaatacaacggcataataacag

SEQ ID NO: 80 - DNA, 23 bp, artificial
  artifical primer
gaaatacaatggcatgataacag

SEQ ID NO: 81 - DNA, 22 bp, artificial
  artifical primer
cctaattatcattacgaggaat

SEQ ID NO: 82 - DNA, 22 bp, artificial
  artifical primer
cctaattatcactatgaggagt

SEQ ID NO: 83 - DNA, 22 bp, artificial
  artifical primer
cctaattaccactatgaggagt

SEQ ID NO: 84 - DNA, 22 bp, artificial
  artifical primer
cctaattaccactatgaggaat

SEQ ID NO: 85 - DNA, 22 bp, artificial
  artifical primer
cctaattatcattaccaggaat

SEQ ID NO: 86 - DNA, 22 bp, artificial
  artifical primer
cctaattatcattacgaagaat

SEQ ID NO: 87 - DNA, 22 bp, artificial
  artifical primer
cctaattatcactatgaggaat

SEQ ID NO: 88 - DNA, 21 bp, artificial
  artifical primer
ggatatatatgcagtggggtt

SEQ ID NO: 89 - DNA, 21 bp, artificial
  artifical primer
ggatatatatgcagtggagtt

SEQ ID NO: 90 - DNA, 21 bp, artificial
  artifical primer
ggatacatatgcagtggggtt

SEQ ID NO: 91 - DNA, 21 bp, artificial
  artifical primer
gggttttcatttaaatacggc

SEQ ID NO: 92 - DNA, 21 bp, artificial
  artifical primer
gggttttcatttaagtatggc

SEQ ID NO: 93 - DNA, 21 bp, artificial
  artifical primer
gggttttcatttaaatatggc

SEQ ID NO: 94 - DNA, 23 bp, artificial
  artifical primer
ctgttattatgccattgtatttc

SEQ ID NO: 95 - DNA, 23 bp, artificial
  artifical primer
ctgttatcatgccattgtatttc

SEQ ID NO: 96 - DNA, 23 bp, artificial
  artifical primer
ctgttattatgccgttgtatttc

SEQ ID NO: 97 - DNA, 22 bp, artificial
  artifical primer
attcctcgtaatgataattagg

SEQ ID NO: 98 - DNA, 22 bp, artificial
  artifical primer
actcctcatagtgataattagg

SEQ ID NO: 99 - DNA, 22 bp, artificial
  artifical primer
actcctcatagtggtaattagg

SEQ ID NO: 100 - DNA, 22 bp, artificial
  artifical primer
attcctcatagtggtaattagg

SEQ ID NO: 101 - DNA, 22 bp, artificial
  artifical primer
attcctggtaatgataattagg

SEQ ID NO: 102 - DNA, 22 bp, artificial
  artifical primer
attcttcgtaatgataattagg

SEQ ID NO: 103 - DNA, 22 bp, artificial
  artifical primer
attcctcatagtgataattagg

SEQ ID NO: 104 - DNA, 21 bp, artificial
  artifical primer
gtctccgaaaaccccactgca

SEQ ID NO: 105 - DNA, 21 bp, artificial
  artifical primer
gtctccgaaaactccactgca

SEQ ID NO: 106 - DNA, 21 bp, artificial
  artifical primer
gccgtatttaaatgaaaaccc

SEQ ID NO: 107 - DNA, 21 bp, artificial
  artifical primer
gccatacttaaatgaaaaccc

SEQ ID NO: 108 - DNA, 21 bp, artificial
  artifical primer
gccatatttaaatgaaaaccc

SEQ ID NO: 109 - DNA, 25 bp, artificial
  artifical primer
ctcccgctrtakcctgaccaatcag

SEQ ID NO: 110 - DNA, 25 bp, artificial
  artifical primer
ctcccgctatatcctgaccaatcag

SEQ ID NO: 111 - DNA, 25 bp, artificial
  artifical primer
ctcccgctatagcctgaccaatcag

SEQ ID NO: 112 - DNA, 26 bp, artificial
  artifical primer
gggactcaacaattatgaaaagtgaa

SEQ ID NO: 113 - DNA, 25 bp, artificial
  artifical primer
ggcatgtgtaacagtaacgttcttt

SEQ ID NO: 114 - DNA, 24 bp, artificial
  artifical primer
cattggagtttgacacttggtgtt

SEQ ID NO: 115 - DNA, 28 bp, artificial
  artifical primer
aactcgacagagcaggttgacacaataa

SEQ ID NO: 116 - DNA, 30 bp, artificial
  artifical primer
tatgaaaagtgaattggaatatggtaactg

SEQ ID NO: 117 - DNA, 31 bp, artificial
  artifical primer
tggaatatggtaactgcaacaccaagtgtca

SEQ ID NO: 118 - DNA, 20 bp, artificial
  artificial primer
tctgggccagaaattccaat

SEQ ID NO: 119 - DNA, 24 bp, artificial
  artificial primer
agtgcttgccatgatggcaccagt